(19) United States
(12) Patent Application Publication
(10) Pub. No.: US 2023/0048847 A1
(43) Pub. Date: Feb. 16, 2023

(54) ENGINEERED LEISHMANIA CELLS

(71) Applicant: LIMMATECH BIOLOGICS AG, Schlieren (CH)

(72) Inventors: Manuela MALLY, Schlieren (CH); Amirreza FARIDMOAYER, Schlieren (CH); Fabio SERVENTI, Schlieren (CH); Rainer FOLLADOR, Schlieren (CH); Anke Judith HARSMAN, Schlieren (CH)

(73) Assignee: LIMMATECH BIOLOGICS AG, Schlieren (CH)

(21) Appl. No.: 17/791,085

(22) PCT Filed: Jan. 7, 2021
(86) PCT No.: PCT/EP2021/050173

§ 371 Date: Jul. 6, 2022

Related U.S. Application Data
(60) Provisional application No. 62/958,088, filed on Jan. 7, 2020.

(51) Int. Cl. C12N 15/90; C12N 15/79; C12P 21/00

(52) U.S. Cl. CPC C12N 15/902; C12N 15/79; C12P 21/005

(57) ABSTRACT

The present application relates to a method of recombinantly engineering a Leishmania cell that involves homologous recombination of DNA fragments. Further provided herein are Leishmania cells recombinantly engineered using the method provided herein. Also provided herein are methods of making a polypeptide using a Leishmania cell described herein and polypeptides produced by the methods provided herein.

This application claims priority to U.S. Provisional Application No. 62/958,088, filed on Jan. 7, 2020, the entirety of which is incorporated herein by reference.

### SEQUENCE LISTING

This application incorporates by reference in its entirety the Computer Readable Form (CRF) of a Sequence Listing in ASCII text format. The Sequence Listing text file is entitled “14197-011-228_SEQ_LISTING,” was created on Dec. 21, 2020, and is 1,115,773 bytes in size.

### 1. INTRODUCTION

The present application relates to a method of recombinantly engineering a Leishmania cell that involves homologous recombination of DNA fragments. Further provided herein are Leishmania cells recombinantly engineered using the method provided herein. Also provided herein are methods of making a polypeptide using a Leishmania cell described herein and polypeptides produced by the methods provided herein.

### 2. BACKGROUND

Leishmania sp. have unusual genetic properties, for example, a certain level of lacking transcriptional control. Genes are transcribed into polycistronic pre-mRNAs that are subsequently processed into mature mRNAs by trans splicing, which involves the addition of a spliced leader or miniexon, and polyadenylation. Control of gene expression does not occur at the transcriptional level but rather at the level of RNA stability, translation and protein turnover (Roberts, Sigrid C. (2011) Bioeng Bugs 2 (6), pp. 320-326). These processes are influenced by the non-coding DNA regions (intergenic regions, IRs) between the genes (Breitling, et al. (2002) Protein Expr. Purif. 25 (2), pp. 209-218). For this reason, all protein-coding sequences need to be separated by intergenic regions that may be originating from Leishmania tarentolae or closely related species. This is also the case when using recombinant DNA and vector plasmids.

Direct assembly of multiple linear DNA fragments via homologous recombination, also described as in vivo assembly or transformation associated recombination, has been successfully applied to assemble DNA constructs ranging in size from a few kilobases to full synthetic microbial genomes. It has also enabled the complete replacement of eukaryotic chromosomes with heterologous DNA. Complex in vivo assembly of multiple DNA fragments is a routine procedure using S. cerevisiae, contributing to its extensive use as a synthetic biology and biotechnology host (Shao, et al. (2009) Nucleic acids research 37 (2), e16).

Leishmania sp. effectively undergo homologous recombination, which is used for example to exchange target genes with drug resistance markers where the introduced drug resistance markers provide a selection mechanism. Targeting constructs are designed in which upstream and downstream regions corresponding to the flanking sequences of the target gene are joined to a drug resistance cassette. Previously, time-consuming cloning steps were involved in the generation of targeting DNAs (Roberts, Sigrid C. (2011) Bioeng Bugs 2 (6), pp. 320-326). Some techniques have been developed that simultaneously assemble multiple DNA fragments and considerably simplify the assembly of targeting constructs. Examples include the use of a PCR fusion-based strategy (Mukherjee, et al. (2009) Mol Microbiol 74 (4), pp. 914-927) and a one-step multi-fragment ligation technique (Fulwiler, et al. (2011) Molecular and Biochemical Parasitology 175 (2), pp. 209-212). The general strategy was described to be adaptable to the generation of targeting constructs for other parasites and genetically manipulatable organisms by simply producing species-specific selectable markers flanked by the appropriate SfiI sites (Fulwiler, et al. (2011) Molecular and Biochemical Parasitology 175 (2), pp. 209-212). This multi-fragment ligation technique was described for generating deletion strains, but not for knock-in/insertion strains.

To use Leishmania as an expression host for glycoengineered therapeutic proteins (International Publication No. WO2019/002512 A2, incorporated by reference in its entirety herein), several recombinant elements may be inserted into the host cell genome and co-expressed at the same time. Regulatory DNA sequences flanking the recombinantly expressed genes of interest are required for efficient expression, i.e. for processing and splicing of the mature processed mRNA from a polycistronic pre-mRNA. The number of genes and regulatory sequences to be inserted becomes limiting in the case of multiple gene insertions, because inserting identical sequences into the same genome can lead to undesired recombination events. Provided herein are methods to address these concerns.

### 3. SUMMARY OF THE INVENTION

Provided herein are methods of recombinantly engineering a Leishmania cell, Leishmania cells, kits comprising the Leishmania cells, methods of making a polypeptide using a Leishmania cell, and polypeptides produced by such methods.

In one aspect, provided herein is a method of recombinantly engineering a Leishmania cell comprising

(a) introducing two or more DNA fragments into the Leishmania cell, and
(b) incubating the Leishmania cell to allow homologous recombination of the DNA fragments,
wherein a first DNA fragment of the two or more DNA fragments comprises a 5′ homologous region and/or a 3′ homologous region; wherein the 5′ homologous region is homologous to a 3′ homologous region of a second DNA fragment of the two or more DNA fragments or the 3′ homologous region of the first DNA fragment is homologous to a 5′ homologous region of the second DNA fragment; and
wherein the nucleotide sequences of the first and the second DNA fragments outside the homologous region(s) are not homologous to each other; are not homologous to a sequence in the Leishmania cell's genome; and/or have no homologies within the respective DNA fragment.

In certain embodiments, each of the two or more DNA fragments comprises a 5′ homologous region and/or a 3′ homologous region; wherein the 5′ homologous region of the each of the two or more DNA fragments is homologous to a 3′ homologous region of another one of the two or more DNA fragments or the 3′ homologous region of the each of the two or more DNA fragments is homologous to a 5′ homologous region of another one of the two or more DNA fragments; and wherein the nucleotide sequences outside the homologous regions in each DNA fragment are not homologous to each other; are not homologous to a sequence in the Leishmania cell's genome; and/or have no homologies within the respective DNA fragment.

In certain embodiments, the two or more DNA fragments, optionally after the two or more DNA fragments are recombined with each other, are suitable for integration into a chromosome of the Leishmania cell. In certain embodiments, the two or more DNA fragments, optionally after the two or more DNA fragments are recombined with each other, are integrated into the chromosome of the Leishmania cell. In certain embodiments, the two or more DNA fragments are integrated in tandem into the paraflagellar rod protein (Pfr) locus. In certain embodiments, the two or more DNA fragments are integrated at the start of the 18S coding region (Ssu-PolI).

In certain embodiments, the two or more DNA fragments, before and/or after recombination with each other, are not integrated in a chromosome of the Leishmania cell.

In certain embodiments, the homologous recombination of the two or more DNA fragments results in a circular plasmid.

In certain embodiments, the nucleotide sequence of the first DNA fragment outside the homologous region is at least 10 nucleotides, 20 nucleotides, 30 nucleotides, 40 nucleotides, 50 nucleotides, 100 nucleotides, 200 nucleotides, 300 nucleotides, 400 nucleotides, 500 nucleotides, 600 nucleotides, 700 nucleotides, 800 nucleotides, 900 nucleotides, 1000 nucleotides, 2000 nucleotides, 5000 nucleotides, 10000 nucleotides, 15000 nucleotides, or 20000 nucleotides, 25000 nucleotides, 30000 nucleotides, 35000 nucleotides, 40000 nucleotides, 45000 nucleotides, or at least 50000 nucleotides in length. In certain embodiments, the nucleotide sequence of the second DNA fragment outside the homologous region is at least 10 nucleotides, 20 nucleotides, 30 nucleotides, 40 nucleotides, 50 nucleotides, 100 nucleotides, 200 nucleotides, 300 nucleotides, 400 nucleotides, 500 nucleotides, 600 nucleotides, 700 nucleotides, 800 nucleotides, 900 nucleotides, 1000 nucleotides, 2000 nucleotides, 5000 nucleotides, 10000 nucleotides, 15000 nucleotides, or 20000 nucleotides, 25000 nucleotides, 30000 nucleotides, 35000 nucleotides, 40000 nucleotides, 45000 nucleotides, or at least 50000 nucleotides in length. In certain embodiments, the nucleotide sequences of all of the two or more DNA fragments outside the homologous region are at least 10 nucleotides, 20 nucleotides, 30 nucleotides, 40 nucleotides, 50 nucleotides, 100 nucleotides, 200 nucleotides, 300 nucleotides, 400 nucleotides, 500 nucleotides, 600 nucleotides, 700 nucleotides, 800 nucleotides, 900 nucleotides, 1000 nucleotides, 2000 nucleotides, 5000 nucleotides, 10000 nucleotides, 15000 nucleotides, or 20000 nucleotides, 25000 nucleotides, 30000 nucleotides, 35000 nucleotides, 40000 nucleotides, 45000 nucleotides, or at least 50000 nucleotides in length.

In certain embodiments, the homologous recombination of the DNA fragments results in a nucleotide sequence that is 50 nucleotides to 100 nucleotides, 100 nucleotides to 500 nucleotides, 500 nucleotides to 1000 nucleotides, 1000 nucleotides to 5000 nucleotides, 5000 nucleotides to 10000 nucleotides, 10000 nucleotides to 15000 nucleotides, 15000 nucleotides to 20000 nucleotides, 20000 nucleotides to 25000 nucleotides, 25000 nucleotides to 30000 nucleotides, 30000 nucleotides to 35000 nucleotides, 35000 nucleotides to 40000 nucleotides, 40000 nucleotides to 45000 nucleotides, 45000 nucleotides to 50000 nucleotides, 50000 nucleotides to 55000 nucleotides, 55000 nucleotides to 60000 nucleotides, 60000 nucleotides to 65000 nucleotides, 65000 nucleotides to 70000 nucleotides, 70000 nucleotides to 75000 nucleotides, or 75000 nucleotides to 80000 nucleotides in length.

In certain embodiments, the 5′ homologous region and/or the 3′ homologous region of the first DNA fragment is at least 10 nucleotides, 20 nucleotides, 30 nucleotides, 40 nucleotides, 50 nucleotides, 100 nucleotides, 150 nucleotides, 200 nucleotides, 250 nucleotides, 300 nucleotides, 350 nucleotides, 400 nucleotides, 450 nucleotides, or at least 500 nucleotides in length. In certain embodiments, the 5′ homologous region and/or the 3′ homologous region of the second DNA fragment is at least 10 nucleotides, 20 nucleotides, 30 nucleotides, 40 nucleotides, 50 nucleotides, 100 nucleotides, 150 nucleotides, 200 nucleotides, 250 nucleotides, 300 nucleotides, 350 nucleotides, 400 nucleotides, 450 nucleotides, or at least 500 nucleotides in length. In certain embodiments, the 5′ homologous region and/or the 3′ homologous region of all of the two or more DNA fragments is at least 10 nucleotides, 20 nucleotides, 30 nucleotides, 40 nucleotides, 50 nucleotides, 100 nucleotides, 150 nucleotides, 200 nucleotides, 250 nucleotides, 300 nucleotides, 350 nucleotides, 400 nucleotides, 450 nucleotides, or at least 500 nucleotides in length.

In certain embodiments, the 5′ homologous region and/or the 3′ homologous region of the first DNA fragment is at most 500 nucleotides, 550 nucleotides, 600 nucleotides, 650 nucleotides, 700 nucleotides, 750 nucleotides, 800 nucleotides, 850 nucleotides, 900 nucleotides, 950 nucleotides, 1000 nucleotides, 1200 nucleotides, 1400 nucleotides, 1600 nucleotides, 1800 nucleotides, 2000 nucleotides, 2200 nucleotides, 2400 nucleotides, 2600 nucleotides, 2800 nucleotides, 3000 nucleotides, 3200 nucleotides, 3400 nucleotides, 3600 nucleotides, 3800 nucleotides, 4000 nucleotides, 4200 nucleotides, 4400 nucleotides, 4600 nucleotides, 4800 nucleotides, or at most 5000 nucleotides in length. In certain embodiments, the 5′ homologous region and/or the 3′ homologous region of the second DNA fragment is at most 500 nucleotides, 550 nucleotides, 600 nucleotides, 650 nucleotides, 700 nucleotides, 750 nucleotides, 800 nucleotides, 850 nucleotides, 900 nucleotides, 950 nucleotides, 1000 nucleotides, 1200 nucleotides, 1400 nucleotides, 1600 nucleotides, 1800 nucleotides, 2000 nucleotides, 2200 nucleotides, 2400 nucleotides, 2600 nucleotides, 2800 nucleotides, 3000 nucleotides, 3200 nucleotides, 3400 nucleotides, 3600 nucleotides, 3800 nucleotides, 4000 nucleotides, 4200 nucleotides, 4400 nucleotides, 4600 nucleotides, 4800 nucleotides, or at most 5000 nucleotides in length. In certain embodiments, the 5′ homologous region and/or the 3′ homologous region of all of the two or more DNA fragments is at most 500 nucleotides, 550 nucleotides, 600 nucleotides, 650 nucleotides, 700 nucleotides, 750 nucleotides, 800 nucleotides, 850 nucleotides, 900 nucleotides, 950 nucleotides, 1000 nucleotides, 1200 nucleotides, 1400 nucleotides, 1600 nucleotides, 1800 nucleotides, 2000 nucleotides, 2200 nucleotides, 2400 nucleotides, 2600 nucleotides, 2800 nucleotides, 3000 nucleotides, 3200 nucleotides, 3400 nucleotides, 3600 nucleotides, 3800 nucleotides, 4000 nucleotides, 4200 nucleotides, 4400 nucleotides, 4600 nucleotides, 4800 nucleotides, or at most 5000 nucleotides in length.

In certain embodiments, the 5′ homologous region of the first DNA fragment and the 3′ homologous region of the second DNA fragment have at least 20%, 25%, 30%, 35%, 40%, 45%, 50%, 55%, 60%, 65%, 70%, 75%, 80%, 85%, 90%, 95%, or 100% sequence identity. In certain embodiments, the 3′ homologous region of the first DNA fragment and the 5′ homologous region of the second DNA fragment have at least 20%, 25%, 30%, 35%, 40%, 45%, 50%, 55%, 60%, 65%, 70%, 75%, 80%, 85%, 90%, 95%, or 100% sequence identity.

In certain embodiments, the two or more DNA fragments are introduced by transfection. In certain embodiments, the two or more DNA fragments are introduced concurrently.

In certain embodiments, the number of DNA fragments is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 37, 39, 40, 41, 42, 43, 44, 45, 46, 47, 48, 49, or 50.

In certain embodiments, the nucleotide sequences of the two or more DNA fragments outside the homologous region are selected from a group consisting of intergenic regions (IRs), untranslated regions (UTRs), and open reading frames (ORFs) encoding polypeptides. In certain embodiments, the IRs, UTRs and ORFs are devoid of homologous sequences within itself, and/or homologous sequences to one another.

In certain embodiments, the nucleotide sequences of the two or more DNA fragments outside the homologous region encode the same polypeptide. In certain embodiments, the Leishmania cell is capable of expressing two or more copies of the same polypeptide. In certain embodiments, the method increases the expression level of the polypeptide.

In certain embodiments, the homologous recombination of the DNA fragments results in a nucleotide sequence comprising at least 50%, 60%, 70%, 80%, 90% or 100% of genetic information encoded by the two or more DNA fragments.

In certain embodiments, the undesired crossing out and/or crossing over occurs in at most 0.01%, 0.05%, 0.1%, 0.5%, 1%, 2%, 3%, 4%, 5%, 6%, 7%, 8%, 9% or at most 10% of the Leishmania cells over a period of at least 1 day, 2 days, 3 days, 4 days, 5 days, 6 days, 7 days, 8 days, 9 days, or at least 10 days. In certain embodiments, the undesired crossing out and/or crossing over occurs in at most 0.01%, 0.05%, 0.1%, 0.5%, 1%, 2%, 3%, 4%, 5%, 6%, 7%, 8%, 9% or at most 10% of the Leishmania cells over at least 1, 2, 3, 4, 5, 6, 7, 8, 9, or at least 10 cell divisions.

In certain embodiments, the Leishmania cell is Leishmania tarentolae.

In another aspect, provided herein is a Leishmania cell recombinantly engineered using the methods provided herein. In certain embodiments, the Leishmania cell is recombinantly engineered using the method repeatedly. In certain embodiments, the Leishmania cell is Leishmania tarentolae.

In another aspect, provided herein is a kit comprising one or more containers and instructions for use, wherein said one or more containers comprise the Leishmania cell provided herein.

In another aspect, provided herein is a method of making a polypeptide comprising (a) culturing the Leishmania cell provided herein under suitable conditions for polypeptide production; and (b) isolating the polypeptide. In certain embodiments, the method further comprises introducing a nucleotide sequence encoding the polypeptide.

In yet another aspect, provided herein is a polypeptide produced by the method of making a polypeptide provided herein.

### 3.1 Definitions

As used herein and unless otherwise indicated, the term “extremity” refers to a region at the 5′ or 3′ end of a DNA fragment.

As used herein and unless otherwise indicated, the term “about,” when used in conjunction with a number, refers to any number within ±1, ±5 or ±10% of the referenced number.

As used herein and unless otherwise indicated, the term “subject” refers to an animal (e.g., birds, reptiles, and mammals). In another embodiment, a subject is a mammal including a non-primate (e.g., a camel, donkey, zebra, cow, pig, horse, goat, sheep, cat, dog, rat, and mouse) and a primate (e.g., a monkey, chimpanzee, and a human). In certain embodiments, a subject is a non-human animal. In some embodiments, a subject is a farm animal or pet (e.g., a dog, cat, horse, goat, sheep, pig, donkey, or chicken). In a specific embodiment, a subject is a human. The terms “subject” and “patient” may be used herein interchangeably.

As used herein and unless otherwise indicated, the term “effective amount,” in the context of administering a therapy (e.g., a composition described herein) to a subject refers to the amount of a therapy which has a prophylactic and/or therapeutic effect(s). In certain embodiments, an “effective amount” refers to the amount of a therapy which is sufficient to achieve one, two, three, four, or more of the following effects: (i) reduce or ameliorate the severity of a disease/disorder or symptom associated therewith; (ii) reduce the duration of a disease/disorder or symptom associated therewith; (iii) prevent the progression of a disease/disorder or symptom associated therewith; (iv) cause regression of a disease/disorder or symptom associated therewith; (v) prevent the development or onset of a disease/disorder, or symptom associated therewith; (vi) prevent the recurrence of a disease/disorder or symptom associated therewith; (vii) reduce organ failure associated with a disease/disorder; (viii) reduce hospitalization of a subject having a disease/disorder; (ix) reduce hospitalization length of a subject having a disease/disorder; (x) increase the survival of a subject with a disease/disorder; (xi) eliminate a disease/disorder in a subject; and/or (xii) enhance or improve the prophylactic or therapeutic effect(s) of another therapy.

### 3.2 Conventions and Abbreviations

| Abbreviation | Convention |
|---|---|
| bp | base pair |
| CDS | coding sequence |
| EPO | erythropoietin |
| GlcNAc-transferase | N-acetylglucosamine-transferase |
| IR | Intergenic region |
| mAb | monoclonal antibody |
| ORF | open reading frames |
| UTR | untranslated region |
| Man3 | 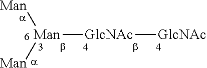 |
| G0-N | 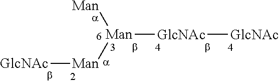 |
| G0 | 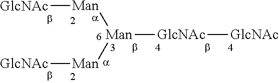 |
| G1-N | 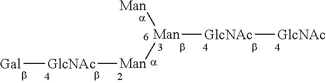 |
| G1 | 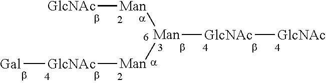 |
| | or |
| | 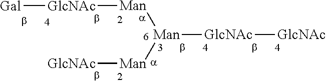 |
| G1S1-N | 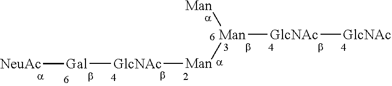 |
| G2 | 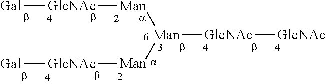 |
| G1S1 | 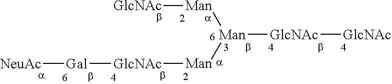 |
| | or |
| | 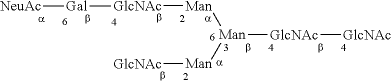 |
| G2S1 | 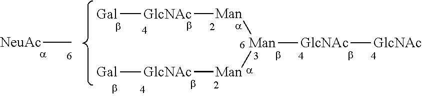 |
| G2S2 | 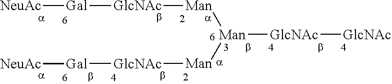 |

### 4. BRIEF DESCRIPTION OF THE FIGURES

FIGS. 1A-1B: Leishmania tarentolae is able to assemble a chromosomal integration construct from multiple DNA fragments by homologous recombination. (FIG. 1A) Schematic representation of a full length monoclonal antibody (mAb) expression construct. The integration construct for Rituximab (top) comprises the homologous recombination sites for integration into the ssu locus (Lhr [ssu] and Rhr [ssu]), 4 intergenic regions ensuring correct transcription and splicing of the mRNA (IR1=aprtlR; IR2=aTubIR from L. enrietti; IR3=CamIR from L. tarentolae; IR4=dhfr-ts) and the open reading frames (ORF) for the light (ORF1) and heavy (ORF2) chains of Rituximab and the selection marker NTC (ORFS). The full construct is present on plasmid pLMTB5026, or split between fragments present on donor plasmids pLMTB5024 and pLMTB5025. Overlapping regions for homologous recombination into the genome (>500 bp; lower part of figure) and between the fragments (250 bp) are indicated as grey bars. (FIG. 1B) Western blot analysis of cell culture supernatants of strains generated by co-transfecting two DNA fragments that should recombine in vivo in order to form an expression construct for a monoclonal antibody (Rituximab). Positive control=Rituximab (Anti CD20, Lubio: A1049-100). Expression of full-length monoclonal antibody can be detected via light chain or heavy chain specific antibodies directly in the cell culture supernatant.

FIG. 2: Multiple homologous recombination events of heterologous coding sequences lead to function-engineered Leishmania host cells. Schematic representation of the integration constructs (top) comprising the homologous recombination sites for integration into the AQP locus (Lhr [AQP] and Rhr [AQP]), regulatory elements (PolA; promoter) and intergenic regions ensuring correct transcription and splicing of the mRNA (aTubIR from L. enrietti; Pfr IR=IR of paraflagellar rod protein from L. tarentolae; Val IR, IR from Valosin from L. tarentolae; Cam IR from L. tarentolae and UtrA=dhfr-ts) and the coding sequences for heterologous glycosyltransferases (sfGntI, rnMGAT2, drMGAT1, hsB4GalT1, see, e.g., International Publication No. WO2019/002512 A2, incorporated by reference in its entirety herein) and the selection marker hygromycin Sm[hyg]). The full construct is split between ten fragments, which were excised from ten donor plasmids. Overlapping regions for homologous recombination into the genome (500 bp) are within black boxes at the extremities and homologous recombination regions between the fragments (200 bp) are indicated as grey bars. Bottom graph shows the functional read out of glycoengineering efficiencies, indicated as relative % N-glycans, which were released from cellular proteins and measured by routine N-glycan analysis such as RF-MS or PC-labeling.

FIGS. 3A-3C: Multiple homologous events of heterologous coding sequences interspaced by Leishmania tarentolae regulatory elements and intergenic regions (IRs). (FIG. 3A) Functional read out of glycoengineering efficiencies is indicated as relative % N-glycans, released from cellular proteins and measured by routine RF-MS, and shows activity of all enzymatic steps in St15368. Absent activities from second glycoengineering enzymatic step by MGAT2 in St15448 suggest phenotypic differences based on desired and undesired integration events. (FIG. 3B) Schematic representation of the integration (top) comprising the homologous recombination sites for counterclockwise disruption of aquaporin (AQP) (dark grey boxes, Lhr and Rhr), regulatory elements PolA, and intergenic regions (striped boxes) ensuring correct transcription and splicing of the mRNA (aTubIR from L. enrietti; Pfr IR=IR of paraflagellar rod protein from L. tarentolae; Val IR, IR from Valosin from L. tarentolae; 60S ribosomal protein L23 from L. tarentolae and a 3′ UTR downstream of the selection marker gene (SmA). The coding sequences for heterologous glycosyltransferases are rnMGAT2 (GtD), hsB4GalT1 (GtE), sfGntI (GtA), drMGAT1 (GtB), and SmA for the selection marker hygromycin. Inserted genetic element regions are shaded with grey dotted background. Correct integration is exemplified for St15368 (top). Region marked with black background and white dots shows the Pfr IR that caused an undesired crossing over to the identical Pfr IR sequence in Chromosome 29, thereby omitting the recombinant genetic elements of rnMGAT2 (GtD), hsB4GalT1 (GtE) in St15448 (bottom) and (FIG. 3C) leading to a hybrid chromosome as identified by PacBio sequencing (PacBio raw subread m54073_181001_130829/9307006/0_32110).

FIG. 4: Schematic representation of the intended integration comprising the 500 bp homologous recombination sites (dark grey boxes) for counterclockwise disruption of Ptr1 in Chromosome 23 (Lhr and Rhr), regulatory element PolI (“PolA”), and intergenic regions (IRs, striped boxes) ensuring correct transcription and splicing of the mRNA and a 3′ UTR downstream of the selection marker gene (SmA). The coding sequences for heterologous glycosyltransferases are hsB4GalT1 (ORF1), hsMGAT1 (ORF2) rnMGAT2 (ORF3), and SM for the selection marker hygromycin. The full expression cassette is split between eight fragments, which were excised from their donor plasmids. Overlapping regions for homologous recombination into the genome (500 bp at the extremities) and between the fragments (200 bp) are indicated as grey boxes. Within black brackets, the region shaded dark grey with white dots marks an identical stretch of 93 bp derived from the 3×HA tag at the C-terminus of hsMGAT1 ORF in donor fragment GtC_5IrLmM(8081), and the identical sequence in IrLmO_5GtD (8085) derived from the 3×HA tag also present at the C-terminus of rnMGAT2 ORF. Homologous recombination of these fragments creates an undesired crossing out and omission of genetic information for IR2 and rnMGAT2. The phenotype is represented by glycoengineering activities of the GTs, and the absence of G0 and G2 glycans suggests absence of MGAT2 activity, shown as graph with relative % N-glycans (Top left).

FIG. 5: Schematic representation of different chromosomal integration strategies with light grey arrows indicating chromosomal coding sequences and dark grey arrows heterologous coding sequences that are inserted via homology ends at their extremities (shaded grey). The regulatory element PolI is a promoter region for PolI transcription and used for transcription initiation in counter clockwise integration constructs.

FIGS. 6A-6B: Heterologous and non-identical sequences ensure correct chromosomal integrations and internal fragment recombinations, while selected heterologous regulatory sequences are functional in Leishmania tarentolae CustomGlycan host cells. (FIG. 6A) Glycoengineering activities of the GTs are assessed as relative % N-glycans derived from total surface glycoproteins and compared between the different strains differing in their IRs but not in the GT and SM coding sequences. (FIG. 6B) Schematic representation of the Nanopore sequencing analysis results for the different integrations and resulting strains (St17212=LmIR, St17311=LdIR, St17176=LiIR, St17180=LmxIR). Each integration comprises the homologous recombination sites for integration into the GP63 locus of Chromosome 10 (LhrD [GP63] and RhrD [GP63]). The intergenic regions (Ir) derived from different Leishmania species L. major (Lm), L. donovani (Ld), L. infantum (Li), L. mexicana (Lmx) and ensuring correct transcription and splicing of the mRNA, are shown as striped boxes with indicated text description for each IR. The coding sequences for heterologous glycosyltransferases, GTs, (sfGntI, rnMGAT2, drMGAT1, hsB4GalT1) are depicted as white arrows and the selection marker hygromycin in grey and are identical in all four strains.

FIG. 7: Stepwise increase of N-glycan conversion efficiency by transfection of several genetic modules, shown as N-glycans (relative in %) from surface glycoproteins of three consecutively generated strains, St17238 (1^{st}), St17294 (2^{nd}) and St17826 (3^{rd}). Increase in copy numbers of glycosyltransferases was achieved by using codon diversified enzymes and homologs from different species (hs, Homo sapiens; rn, Rattus norvegicus, dr, Danio rerio, gj, Gekko japonicus, ag, Anopheles gambiae).

FIGS. 8A-8C: Generation of an N-glycan sialylation proficient cell line St17527 by multiple homologous recombination of 13 fragments into the parental glycosyltransferase containing cell line (St17311). (FIG. 8A) Schematic representation of the genomic modification of St17527. Top indicates genomically integrated expression cassette in gp63 locus (Chromosome 10) from parental cell line St17311. The new integration comprises the homologous recombination sites for the alpha tubulin locus of Chromosome 13 (Lhr [aTub] and Rhr [aTub]), the intergenic regions (Ir) derived from L. infantum (IrLi) and L. major (IrLm) for transcription and splicing of the mRNA, shown as striped boxes. The coding sequences for sialic acid (Neu5Ac) biosynthesis, Golgi import and transfer to N-glycans (such as NeuC_{3×Myc}: UDP-N-acetyl glucosamine 2-epimerase, CgNal: N-acetylneuraminic acid lyase favoring N-acetylneuraminic acid synthesis, NeuB_{3xHA}: CMP-sialic acid synthase, _{3xHA}ST6: Beta-galactoside alpha-2,6-sialyltransferase 1, NeuA_{3xHA}: CMP-sialic acid synthetase and CST_{3×myc} CMP-Neu5Ac transporter) are depicted as white arrows and the selection marker, pac, in grey. (FIG. 8B) HPLC traces of DMB labelled total sialic acid (Neu5Ac+CMP-Neu5Ac) and CMP-Neu5Ac extracted from cell pellets of St17527 show presence of Neu5Ac and the activated sugar CMP-Neu5Ac and thus demonstrate functionality of sialic acid precursor biosynthesis. (FIG. 8C) Glycoengineering activities of the GTs is represented as relative % N-glycans derived from total surface glycoproteins. Total galactosylation and total sialylation is also indicated, demonstrating a function-customized L. tarentolae host cell.

FIGS. 9A-9C: Chromosomal integrations of the same glycoengineering construct into different chromosomal loci for high level glycoengineering activity of the expressed glycosyltransferases. (FIG. 9A) Schematic representation of chromosomal integration strategies targeting the Pfr locus on chromosome 29 as well as the rDNA expression locus on chromosome 27. Light grey arrows indicate chromosomal coding sequences and dark grey arrows indicate heterologous coding sequences that are inserted via homology ends at their extremities (shaded grey). Since the depicted chromosomal regions represent multi-copy loci, integration can occur in several different places, as indicated by the differently shaded grey bars. Regulatory elements ensuring correct processing of the pre-mRNA are shown as differently striped boxes flanking the 5′ end of the first integrated heterologous coding sequence in the case of integrations into the rDNA locus (Ssu and Ssu-PolI) and the 3′ end of the last heterologous coding sequence of the integration constructs. (FIG. 9B) Comparison of glycoengineering activities of the GTs encoded by the same G0 integration construct targeted to different chromosomal integration loci (“Pfr”, “Ssu” or “Ssu-PolI”). Shown are relative % N-glycans derived from total surface glycoproteins of the respective strains. (FIG. 9C) Comparison of glycoengineering activities of the GTs encoded by the same G0 integration construct targeted to different variants of the rDNA chromosomal integration locus (“Ssu” vs. “Ssu-PolI”). Shown are relative % N-glycans released from the Fc N-glycosylation site of a coexpressed monoclonal antibody (Adalimumab).

FIGS. 10A-10C: Multiple homologous recombination events of heterologous coding sequences lead to function-engineered Leishmania host cells. (FIG. 10A) Schematic representation of the integration construct (top) comprising the homologous recombination sites for integration into the “Pfr” locus (LhrP and RhrP), intergenic regions ensuring correct transcription and splicing of the mRNA (15 different IRs from L. major (Lm), L. donovani (Ld), L. infantum (Li), L. tarentolae (Lt) and UtrA=dhfr-ts are shown as striped boxes) and the coding sequences for heterologous glycosyltransferases (different orthologs of MGAT1, MGAT2, hsB4GalT1 are shown as white boxes, see, e.g., International Publication No. WO2019/002512 A2, incorporated by reference in its entirety herein), the coding sequences for enzymes of the sialic acid biogenesis and transfer pathway (shown as dark grey boxes) and the selection marker for Puromycin resistance (SmD, shown as light grey box). The full construct is split between 25 fragments, which were excised from 25 donor plasmids. Overlapping regions for homologous recombination into the genome (500 bp) are within black boxes at the extremities and homologous recombination regions between the fragments (200 bp or longer) are indicated as grey bars. Bottom graph shows the functional read out of glycoengineering efficiencies, indicated as relative % N-glycans, which were released from cellular proteins and measured by routine N-glycan analysis (PC-labeling). (FIG. 10B) Increase of N-glycan conversion efficiency by transfection of several genetic modules, shown as N-glycans (relative in %) from total surface glycoproteins of three consecutively generated strains, St18700 (1^{st}), St19084 (2^{nd}) and St19384 (3^{rd}). Increase in copy numbers of glycosyltransferases was achieved by using codon diversified enzymes and homologs from different species. (FIG. 10C) Alternative strains of different genetic composition that allow almost homogeneous N-glycan conversion to G2S2. Figure shows N-glycans (relative in %) from total surface glycoproteins of three alternative strains, St20157, St20208 and St20224.

FIGS. 11A-11D: Assembly of a hybrid prokaryotic gene cluster on an Escherichia coli cosmid in Leishmania tarentolae (FIG. 11A) Schematic representation of the designed fragments and the expected recombination via 200 bp homologous regions shaded in grey or grey striped. (FIG. 11B) Western blot analysis on E. coli DH5a transformed with plasmids isolated from several polyclones for expression of S. pneumoniae serotype 1 polysaccharide. Lane 1: PageRuler™ Prestained Protein Ladder, 10 to 180 kDa (ThermoFischer scientific), lane 2: polyclone 1.2, lane 3: polyclone 1.3, lane 4: polyclone 1.4, lane 5: polyclone 1.5, lane 6: polyclone 1.6, lane 7: polyclone 1.7, lane 8: polyclone 1.8, lane 9: polyclone 2.3. (FIG. 11C) Control restriction of plasmids isolated from different polyclones. A: restrictions on polyclones from transfections #1 and #2. Upper panel: BstBI restriction (expected sizes for correct construct: 22210 bp, 9042 bp, 3787 bp; for empty vector: 20801 bp), lower panel: BsiWI restriction (expected sizes for correct construct: 32536 bp, 2503 bp; for empty vector: undigested 20801 bp). Lane 1: GeneRuler™ 1 kb DNA ladder (Thermo-Fischer scientific), lane 2: polyclone 1.1, lane 3: polyclone 1.7, lane 4: polyclone 2.1, lane 5: polyclone 2.2, lane 6: pGVXN775. (FIG. 11D) Restriction on polyclones from transfection #3. Sad restriction (expected sizes for correct construct: 19628 bp, 3723 bp; for empty vector: 20801 bp). Lane 1: polyclone 3.1, lane 2: polyclone 3.2, lane 3: pLMTB6412, lane 4: GeneRuler™ 1 kb DNA ladder (Thermo-Fischer scientific).

### 5. DETAILED DESCRIPTION OF THE INVENTION

Provided herein are methods of recombinantly engineering a Leishmania cell, Leishmania cells engineered using the methods provided herein, methods of making a target polypeptide using a Leishmania cell provided herein, and target polypeptides produced by the methods. The methods of recombinantly engineering a Leishmania cell are described in Section 5.1. Properties of the resulting Leishmania cell are described in Section 5.2. Uses of such Leishmania cell as expression systems for target polypeptides, e.g., therapeutic proteins, are described in Section 5.3. Properties of the target polypeptides expressed in Leishmania host cells provided herein are described in Section 5.4.

Provided herein are i) a quick, multi-fragment homologous recombination to create large artificial chromosomal insertions of at least around 20 kb in Leishmania tarentolae hosts cells, ii) specific strategies to avoid undesired crossing out of recombinantly inserted genetic elements and iii) using multiple homologous recombination to assemble circular DNA adaptable for any heterologous shuttle vector use. Further, also provided herein is a method of increasing expression of a polypeptide by insertion of multiple expressed gene copies into the same host cell.

Problems using a multiple homologous recombination event for host cell engineering occur if the inserted DNA sequences are homologous/identical. Even short stretches of less than 100 bp can lead to undesired crossing over due to the very efficient natural recombination of Leishmania tarentolae.

Without being bound by theory, the methods provided herein reduce or eliminate such undesired crossing over. Specifically, described herein are i) multiple regulatory DNA sequences that enable functional processing and splicing of polycistronic pre-mRNA to form mature processed mRNA for protein expression, while differing sufficiently from each other and from any chromosomal sequence to avoid undesired crossing over (such sequences were taken from related species but not from Leishmania tarentolae), and ii) strategies to diversify coding sequences for genes that are intended to be inserted in multiple copies in such a way that they are not recombining with themselves but still are efficiently expressed.

The multi-fragment ligation strategy described herein for creating engineered host cells is markedly faster than traditional approaches, it moreover allows simultaneous integration of multiple ORFs with only one selection marker and thus expands the previously limited capacity of genetic engineering possibilities. Furthermore, the selection of different insertion elements (intergenic regulatory sequences or codon-diversified genes of interest) enables expression of glycoengineered therapeutics and yield increases by increasing gene copy numbers. This application describes fully function-customized host cells, which were created by the genetic methods described.

Moreover, due to L. tarentolae very efficient innate homologous recombination system, L. tarentolae can be used to assemble multiple heterologous DNA fragments to a circular DNA, if homologous sites to the L. tarentolae chromosome are absent. L. tarentolae is able to propagate episomal (Plasmid) DNA in absence of origin of replication. These methods consist of co-transfecting the donor plasmid and a series of DNA fragments which share homologies in their extremities and between their extremities and the recipient vector. A selection marker for L. tarentolae is added, which is as well separated into 2 fragments for selecting positive transfectants of L. tarentolae host cells. Nucleic acid from L. tarentolae PCR-positive cells can be extracted and the extracted material is transformed/transfected into target propagating microorganism on desired selection marker present in recipient vector. The technology can use any unmodified recipient circular DNA and no restriction site availability is necessary.

To summarize, multiple homologous recombination events, which efficiently occur in Leishmania, are exploited to introduce 2 to 20, and potentially even far more than 20, DNA fragments, which share homologies in their extremities, in order to create host cells that have site specifically integrated the genetic information containing coding sequences flanked by regulatory elements. Regulatory elements were identified from related species and remain functional when introduced in Leishmania tarentolae. Final genetic information comprises stretches of 20 kb/site specifically inserted into the chromosome of Leishmania host cells, and even larger insertions are possible. The application describes a novel tool using “plug and play” modules to efficiently create function-engineered Leishmania host cells. Additionally this efficient site-specific homologous recombination event is exploited to assemble multiple DNA fragments into an episomal construct, which can be adapted as shuttle vector for different unrelated host organisms, thereby representing an efficient cloning tool for complex shuttle vectors.

### 5.1 Methods of Recombinantly Engineering a Leishmania Cell

In one aspect, provided herein is a method of recombinantly engineering a Leishmania cell comprising (a) introducing two or more DNA fragments into the Leishmania cell, and (b) incubating the Leishmania cell to allow homologous recombination of the DNA fragments, wherein a first DNA fragment of the two or more DNA fragments comprises a 5′ homologous region and/or a 3′ homologous region; wherein the 5′ homologous region is homologous to a 3′ homologous region of a second DNA fragment of the two or more DNA fragments or the 3′ homologous region of the first DNA fragment is homologous to a 5′ homologous region of the second DNA fragment; and wherein the nucleotide sequences of the first and the second DNA fragments outside the homologous region(s) are not homologous to each other; are not homologous to a sequence in the Leishmania cell's genome; and/or have no homologies within the respective DNA fragment.

### 5.1.1 DNA Fragments

In certain embodiments, the DNA fragment described herein comprises a 5′ homologous region or a 3′ homologous region that is homologous to a 5′ homologous region or a 3′ homologous region of another DNA fragment. In certain embodiments, the DNA fragment described herein comprises a 5′ homologous region that is homologous to a 3′ homologous region of another DNA fragment. In certain embodiments, the DNA fragment described herein comprises a 3′ homologous region that is homologous to a 5′ homologous region of another DNA fragment. In certain embodiments, the DNA fragment described herein comprises a 5′ homologous region that is homologous to a 3′ homologous region of another DNA fragment, and a 3′ homologous region that is homologous to a 5′ homologous region of a third DNA fragment. In certain embodiments, the nucleotide sequences outside the homologous regions in the DNA fragment described herein are not homologous to each other. In certain embodiments, the nucleotide sequences outside the homologous regions in the DNA fragment described herein are not homologous to a sequence in the Leishmania cell's genome. In certain embodiments, the nucleotide sequences outside the homologous regions in the DNA fragment described herein have no homologies within the respective DNA fragment.

In certain embodiments, the first DNA fragment of the two or more DNA fragments comprises a 5′ homologous region and/or a 3′ homologous region. In certain embodiments, the 5′ homologous region of the first DNA fragment is homologous to a 3′ homologous region of a second DNA fragment of the two or more DNA fragments. In certain embodiments, the 3′ homologous region of the first DNA fragment is homologous to a 5′ homologous region of a second DNA fragment of the two or more DNA fragments. In certain embodiments, the nucleotide sequences of the first and the second DNA fragments outside the homologous region(s) are not homologous to each other; are not homologous to a sequence in the Leishmania cell's genome; and/or have no homologies within the respective DNA fragment.

In certain embodiments, each of the two or more DNA fragments comprises a 5′ homologous region and/or a 3′ homologous region. In certain embodiments, the 5′ homologous region of the each of the two or more DNA fragments is homologous to a 3′ homologous region of another one of the two or more DNA fragments. In certain embodiments, the 3′ homologous region of the each of the two or more DNA fragments is homologous to a 5′ homologous region of another one of the two or more DNA fragments. In certain embodiments, the nucleotide sequences outside the homologous regions in each DNA fragment are not homologous to each other; are not homologous to a sequence in the Leishmania cell's genome; and/or have no homologies within the respective DNA fragment.

In certain embodiments, the DNA fragment described herein comprises a 5′ homologous region or a 3′ homologous region that is homologous to a region in the chromosome of the Leishmania cell. In certain embodiments, such homologous region allows the integration of the DNA fragment into the chromosome of the Leishmania cell. In certain embodiments, the DNA fragment comprises a 5′ homologous region that is homologous to a 3′ homologous region of another DNA fragment, a region that is outside the homologous regions, and a 3′ homologous region that is homologous to a 5′ homologous region of another DNA fragment.

In certain embodiments, the two or more DNA fragments, optionally after the two or more DNA fragments are recombined with each other, are suitable for integration into a chromosome of the Leishmania cell. In certain embodiments, the two or more DNA fragments, optionally after the two or more DNA fragments are recombined with each other, are integrated into the chromosome of the Leishmania cell. In certain embodiments, the two or more DNA fragments are integrated in tandem into the paraflagellar rod protein (Pfr) locus. In certain embodiments, the two or more DNA fragments are integrated at the start of the 18S coding region (Ssu-PolI). In certain embodiments, the two or more DNA fragments, before and/or after recombination with each other, are not integrated in a chromosome of the Leishmania cell.

### 5.1.2 Homologous Region

In certain embodiments, the 5′ homologous region and/or the 3′ homologous region may be 10 to 2000 nucleotides in length. In certain embodiments, the 5′ homologous region and/or the 3′ homologous region may be at least 10 nucleotides, 20 nucleotides, 30 nucleotides, 40 nucleotides, 50 nucleotides, 60 nucleotides, 70 nucleotides, 80 nucleotides, 90 nucleotides, 100 nucleotides, 150 nucleotides, 200 nucleotides, 250 nucleotides, 300 nucleotides, 350 nucleotides, 400 nucleotides, 450 nucleotides, 500 nucleotides, 550 nucleotides, 600 nucleotides, 650 nucleotides, 700 nucleotides, 750 nucleotides, 800 nucleotides, 850 nucleotides, 900 nucleotides, 950 nucleotides, 1000 nucleotides, 1200 nucleotides, 1400 nucleotides, 1600 nucleotides, 1800 nucleotides, 2000 nucleotides, 2200 nucleotides, 2400 nucleotides, 2600 nucleotides, 2800 nucleotides, 3000 nucleotides, 3200 nucleotides, 3400 nucleotides, 3600 nucleotides, 3800 nucleotides, 4000 nucleotides, 4200 nucleotides, 4400 nucleotides, 4600 nucleotides, 4800 nucleotides, or at least 5000 nucleotides in length. In certain embodiments, the 5′ homologous region and/or the 3′ homologous region may be 10 nucleotides, 20 nucleotides, 30 nucleotides, 40 nucleotides, 50 nucleotides, 60 nucleotides, 70 nucleotides, 80 nucleotides, 90 nucleotides, 100 nucleotides, 150 nucleotides, 200 nucleotides, 250 nucleotides, 300 nucleotides, 350 nucleotides, 400 nucleotides, 450 nucleotides, 500 nucleotides, 550 nucleotides, 600 nucleotides, 650 nucleotides, 700 nucleotides, 750 nucleotides, 800 nucleotides, 850 nucleotides, 900 nucleotides, 950 nucleotides, 1000 nucleotides, 1200 nucleotides, 1400 nucleotides, 1600 nucleotides, 1800 nucleotides, 2000 nucleotides, 2200 nucleotides, 2400 nucleotides, 2600 nucleotides, 2800 nucleotides, 3000 nucleotides, 3200 nucleotides, 3400 nucleotides, 3600 nucleotides, 3800 nucleotides, 4000 nucleotides, 4200 nucleotides, 4400 nucleotides, 4600 nucleotides, 4800 nucleotides, or 5000 nucleotides in length. In certain embodiments, the 5′ homologous region and/or the 3′ homologous region may be 200 nucleotides, 250 nucleotides or more than 500 nucleotides in length. In certain embodiments, the 5′ homologous region and/or the 3′ homologous region may be of any length that is described in the Example section.

In certain embodiments, the 5′ homologous region and/or the 3′ homologous region of the first DNA fragment is at least 10 nucleotides, 20 nucleotides, 30 nucleotides, 40 nucleotides, 50 nucleotides, 100 nucleotides, 150 nucleotides, 200 nucleotides, 250 nucleotides, 300 nucleotides, 350 nucleotides, 400 nucleotides, 450 nucleotides, or at least 500 nucleotides in length. In certain embodiments, the 5′ homologous region and/or the 3′ homologous region of the first DNA fragment is at most 500 nucleotides, 550 nucleotides, 600 nucleotides, 650 nucleotides, 700 nucleotides, 750 nucleotides, 800 nucleotides, 850 nucleotides, 900 nucleotides, 950 nucleotides, 1000 nucleotides, 1200 nucleotides, 1400 nucleotides, 1600 nucleotides, 1800 nucleotides, 2000 nucleotides, 2200 nucleotides, 2400 nucleotides, 2600 nucleotides, 2800 nucleotides, 3000 nucleotides, 3200 nucleotides, 3400 nucleotides, 3600 nucleotides, 3800 nucleotides, 4000 nucleotides, 4200 nucleotides, 4400 nucleotides, 4600 nucleotides, 4800 nucleotides, or at most 5000 nucleotides in length. In certain embodiments, the 5′ homologous region and/or the 3′ homologous region of the first DNA fragment is 10 nucleotide to 50 nucleotides, 50 nucleotide to 100 nucleotides, 100 nucleotide to 150 nucleotides, 150 nucleotide to 200 nucleotides, 200 nucleotide to 250 nucleotides, 250 nucleotide to 300 nucleotides, 300 nucleotide to 350 nucleotides, 350 nucleotide to 400 nucleotides, 400 nucleotide to 450 nucleotides, 450 nucleotide to 500 nucleotides, 500 nucleotide to 550 nucleotides, 550 nucleotide to 600 nucleotides, 600 nucleotide to 650 nucleotides, 650 nucleotide to 700 nucleotides, 700 nucleotide to 750 nucleotides, 750 nucleotide to 800 nucleotides, 800 nucleotide to 850 nucleotides, 850 nucleotide to 900 nucleotides, 900 nucleotide to 950 nucleotides, 950 nucleotide to 1000 nucleotides, 1000 nucleotides to 1200 nucleotides, 1200 nucleotides to 1400 nucleotides, 1400 nucleotides to 1600 nucleotides, 1600 nucleotides to 1800 nucleotides, 1800 nucleotides to 2000 nucleotides, 2000 nucleotides to 2200 nucleotides, 2200 nucleotides to 2400 nucleotides, 2400 nucleotides to 2600 nucleotides, 2600 nucleotides to 2800 nucleotides, 2800 nucleotides to 3000 nucleotides, 3000 nucleotides to 3200 nucleotides, 3200 nucleotides to 3400 nucleotides, 3400 nucleotides to 3600 nucleotides, 3600 nucleotides to 3800 nucleotides, 3800 nucleotides to 4000 nucleotides, 4000 nucleotides to 4200 nucleotides, 4200 nucleotides to 4400 nucleotides, 4400 nucleotides to 4600 nucleotides, 4600 nucleotides to 4800 nucleotides, or 4800 nucleotides to 5000 nucleotides in length.

In certain embodiments, the 5′ homologous region and/or the 3′ homologous region of the second DNA fragment is at least 10 nucleotides, 20 nucleotides, 30 nucleotides, 40 nucleotides, 50 nucleotides, 100 nucleotides, 150 nucleotides, 200 nucleotides, 250 nucleotides, 300 nucleotides, 350 nucleotides, 400 nucleotides, 450 nucleotides, or at least 500 nucleotides in length. In certain embodiments, the 5′ homologous region and/or the 3′ homologous region of the second DNA fragment is at most 500 nucleotides, 550 nucleotides, 600 nucleotides, 650 nucleotides, 700 nucleotides, 750 nucleotides, 800 nucleotides, 850 nucleotides, 900 nucleotides, 950 nucleotides, 1000 nucleotides, 1200 nucleotides, 1400 nucleotides, 1600 nucleotides, 1800 nucleotides, 2000 nucleotides, 2200 nucleotides, 2400 nucleotides, 2600 nucleotides, 2800 nucleotides, 3000 nucleotides, 3200 nucleotides, 3400 nucleotides, 3600 nucleotides, 3800 nucleotides, 4000 nucleotides, 4200 nucleotides, 4400 nucleotides, 4600 nucleotides, 4800 nucleotides, or at most 5000 nucleotides in length. In certain embodiments, the 5′ homologous region and/or the 3′ homologous region of the second DNA fragment is 10 nucleotide to 50 nucleotides, 50 nucleotide to 100 nucleotides, 100 nucleotide to 150 nucleotides, 150 nucleotide to 200 nucleotides, 200 nucleotide to 250 nucleotides, 250 nucleotide to 300 nucleotides, 300 nucleotide to 350 nucleotides, 350 nucleotide to 400 nucleotides, 400 nucleotide to 450 nucleotides, 450 nucleotide to 500 nucleotides, 500 nucleotide to 550 nucleotides, 550 nucleotide to 600 nucleotides, 600 nucleotide to 650 nucleotides, 650 nucleotide to 700 nucleotides, 700 nucleotide to 750 nucleotides, 750 nucleotide to 800 nucleotides, 800 nucleotide to 850 nucleotides, 850 nucleotide to 900 nucleotides, 900 nucleotide to 950 nucleotides, 950 nucleotide to 1000 nucleotides, 1000 nucleotides to 1200 nucleotides, 1200 nucleotides to 1400 nucleotides, 1400 nucleotides to 1600 nucleotides, 1600 nucleotides to 1800 nucleotides, 1800 nucleotides to 2000 nucleotides, 2000 nucleotides to 2200 nucleotides, 2200 nucleotides to 2400 nucleotides, 2400 nucleotides to 2600 nucleotides, 2600 nucleotides to 2800 nucleotides, 2800 nucleotides to 3000 nucleotides, 3000 nucleotides to 3200 nucleotides, 3200 nucleotides to 3400 nucleotides, 3400 nucleotides to 3600 nucleotides, 3600 nucleotides to 3800 nucleotides, 3800 nucleotides to 4000 nucleotides, 4000 nucleotides to 4200 nucleotides, 4200 nucleotides to 4400 nucleotides, 4400 nucleotides to 4600 nucleotides, 4600 nucleotides to 4800 nucleotides, or 4800 nucleotides to 5000 nucleotides in length.

In certain embodiments, the 5′ homologous region and/or the 3′ homologous region of all of the two or more DNA fragments is at least 10 nucleotides, 20 nucleotides, 30 nucleotides, 40 nucleotides, 50 nucleotides, 100 nucleotides, 150 nucleotides, 200 nucleotides, 250 nucleotides, 300 nucleotides, 350 nucleotides, 400 nucleotides, 450 nucleotides, or at least 500 nucleotides in length. In certain embodiments, the 5′ homologous region and/or the 3′ homologous region of all of the two or more DNA fragments is at most 500 nucleotides, 550 nucleotides, 600 nucleotides, 650 nucleotides, 700 nucleotides, 750 nucleotides, 800 nucleotides, 850 nucleotides, 900 nucleotides, 950 nucleotides, 1000 nucleotides, 1200 nucleotides, 1400 nucleotides, 1600 nucleotides, 1800 nucleotides, 2000 nucleotides, 2200 nucleotides, 2400 nucleotides, 2600 nucleotides, 2800 nucleotides, 3000 nucleotides, 3200 nucleotides, 3400 nucleotides, 3600 nucleotides, 3800 nucleotides, 4000 nucleotides, 4200 nucleotides, 4400 nucleotides, 4600 nucleotides, 4800 nucleotides, or at most 5000 nucleotides in length. In certain embodiments, the 5′ homologous region and/or the 3′ homologous region of all of the two or more DNA fragments is 10 nucleotide to 50 nucleotides, 50 nucleotide to 100 nucleotides, 100 nucleotide to 150 nucleotides, 150 nucleotide to 200 nucleotides, 200 nucleotide to 250 nucleotides, 250 nucleotide to 300 nucleotides, 300 nucleotide to 350 nucleotides, 350 nucleotide to 400 nucleotides, 400 nucleotide to 450 nucleotides, 450 nucleotide to 500 nucleotides, 500 nucleotide to 550 nucleotides, 550 nucleotide to 600 nucleotides, 600 nucleotide to 650 nucleotides, 650 nucleotide to 700 nucleotides, 700 nucleotide to 750 nucleotides, 750 nucleotide to 800 nucleotides, 800 nucleotide to 850 nucleotides, 850 nucleotide to 900 nucleotides, 900 nucleotide to 950 nucleotides, 950 nucleotide to 1000 nucleotides, 1000 nucleotides to 1200 nucleotides, 1200 nucleotides to 1400 nucleotides, 1400 nucleotides to 1600 nucleotides, 1600 nucleotides to 1800 nucleotides, 1800 nucleotides to 2000 nucleotides, 2000 nucleotides to 2200 nucleotides, 2200 nucleotides to 2400 nucleotides, 2400 nucleotides to 2600 nucleotides, 2600 nucleotides to 2800 nucleotides, 2800 nucleotides to 3000 nucleotides, 3000 nucleotides to 3200 nucleotides, 3200 nucleotides to 3400 nucleotides, 3400 nucleotides to 3600 nucleotides, 3600 nucleotides to 3800 nucleotides, 3800 nucleotides to 4000 nucleotides, 4000 nucleotides to 4200 nucleotides, 4200 nucleotides to 4400 nucleotides, 4400 nucleotides to 4600 nucleotides, 4600 nucleotides to 4800 nucleotides, or 4800 nucleotides to 5000 nucleotides in length.

In certain embodiments, two homologous regions that are homologous to each other have at least 20%, 25%, 30%, 35%, 40%, 45%, 50%, 55%, 60%, 65%, 70%, 75%, 80%, 85%, 90%, 95%, or 100% sequence identity. In certain embodiments, two homologous regions that are homologous to each other have 20%, 25%, 30%, 35%, 40%, 45%, 50%, 55%, 60%, 65%, 70%, 75%, 80%, 85%, 90%, 95%, or 100% sequence identity. In certain embodiments, two homologous regions have enough level of homology to allow homologous recombination of the corresponding DNA fragments comprising the homologous regions.

In certain embodiments, the 5′ homologous region of the first DNA fragment and the 3′ homologous region of the second DNA fragment have at least 20%, 25%, 30%, 35%, 40%, 45%, 50%, 55%, 60%, 65%, 70%, 75%, 80%, 85%, 90%, 95%, or 100% sequence identity. In certain embodiments, the 3′ homologous region of the first DNA fragment and the 5′ homologous region of the second DNA fragment have at least 20%, 25%, 30%, 35%, 40%, 45%, 50%, 55%, 60%, 65%, 70%, 75%, 80%, 85%, 90%, 95%, or 100% sequence identity. In certain embodiments, the 5′ homologous region of the first DNA fragment and the 3′ homologous region of the second DNA fragment have 20%, 25%, 30%, 35%, 40%, 45%, 50%, 55%, 60%, 65%, 70%, 75%, 80%, 85%, 90%, 95%, or 100% sequence identity. In certain embodiments, the 3′ homologous region of the first DNA fragment and the 5′ homologous region of the second DNA fragment have 20%, 25%, 30%, 35%, 40%, 45%, 50%, 55%, 60%, 65%, 70%, 75%, 80%, 85%, 90%, 95%, or 100% sequence identity.

### 5.1.3 the DNA Fragments Outside the Homologous Region

In certain embodiments, the nucleotide sequences of the DNA fragments outside the homologous region are at least 10 nucleotides, 20 nucleotides, 30 nucleotides, 40 nucleotides, 50 nucleotides, 100 nucleotides, 200 nucleotides, 300 nucleotides, 400 nucleotides, 500 nucleotides, 600 nucleotides, 700 nucleotides, 800 nucleotides, 900 nucleotides, 1000 nucleotides, 1200 nucleotides, 1500 nucleotides, 1800 nucleotides, 2000 nucleotides, 2500 nucleotides, 3000 nucleotides, 3500 nucleotides, 4000 nucleotides, 4500 nucleotides, 5000 nucleotides, 6000 nucleotides, 7000 nucleotides, 8000 nucleotides, 9000 nucleotides, 10000 nucleotides, 11000 nucleotides, 12000 nucleotides, 13000 nucleotides, 14000 nucleotides, 15000 nucleotides, 16000 nucleotides, 17000 nucleotides, 18000 nucleotides, 19000 nucleotides, 20000 nucleotides, 25000 nucleotides, 30000 nucleotides, 35000 nucleotides, 40000 nucleotides, 45000 nucleotides, or at least 50000 nucleotides in length. In certain embodiments, the nucleotide sequences of the DNA fragments outside the homologous region are 10 to 50000 nucleotides in length. In certain embodiments, the nucleotide sequences of the DNA fragments outside the homologous region are 50 to 10000 nucleotides in length. In certain embodiments, the nucleotide sequences of the DNA fragments outside the homologous region are 100 to 5000 nucleotides in length. In certain embodiments, the nucleotide sequences of the DNA fragments outside the homologous region are 150 to 2500 nucleotides in length. In certain embodiments, the nucleotide sequences of the DNA fragments outside the homologous region are 250 to 2000 nucleotides in length.

In certain embodiments, the nucleotide sequence of the first DNA fragment outside the homologous region is at least 10 nucleotides, 20 nucleotides, 30 nucleotides, 40 nucleotides, 50 nucleotides, 100 nucleotides, 200 nucleotides, 300 nucleotides, 400 nucleotides, 500 nucleotides, 600 nucleotides, 700 nucleotides, 800 nucleotides, 900 nucleotides, 1000 nucleotides, 2000 nucleotides, 5000 nucleotides, 10000 nucleotides, 15000 nucleotides, or 20000 nucleotides, 25000 nucleotides, 30000 nucleotides, 35000 nucleotides, 40000 nucleotides, 45000 nucleotides, or at least 50000 nucleotides in length. In certain embodiments, the nucleotide sequences of the first DNA fragment outside the homologous region are 10 to 50000 nucleotides in length. In certain embodiments, the nucleotide sequences of the first DNA fragment outside the homologous region are 50 to 10000 nucleotides in length. In certain embodiments, the nucleotide sequences of the first DNA fragment outside the homologous region are 100 to 5000 nucleotides in length. In certain embodiments, the nucleotide sequences of the first DNA fragment outside the homologous region are 150 to 2500 nucleotides in length. In certain embodiments, the nucleotide sequences of the first DNA fragment outside the homologous region are 250 to 2000 nucleotides in length.

In certain embodiments, the nucleotide sequence of the second DNA fragment outside the homologous region is at least 10 nucleotides, 20 nucleotides, 30 nucleotides, 40 nucleotides, 50 nucleotides, 100 nucleotides, 200 nucleotides, 300 nucleotides, 400 nucleotides, 500 nucleotides, 600 nucleotides, 700 nucleotides, 800 nucleotides, 900 nucleotides, 1000 nucleotides, 2000 nucleotides, 5000 nucleotides, 10000 nucleotides, 15000 nucleotides, or 20000 nucleotides, 25000 nucleotides, 30000 nucleotides, 35000 nucleotides, 40000 nucleotides, 45000 nucleotides, or at least 50000 nucleotides in length. In certain embodiments, the nucleotide sequences of the second DNA fragment outside the homologous region are 10 to 50000 nucleotides in length. In certain embodiments, the nucleotide sequences of the first DNA fragment outside the homologous region are 50 to 10000 nucleotides in length. In certain embodiments, the nucleotide sequences of the second DNA fragment outside the homologous region are 100 to 5000 nucleotides in length. In certain embodiments, the nucleotide sequences of the second DNA fragment outside the homologous region are 150 to 2500 nucleotides in length. In certain embodiments, the nucleotide sequences of the second DNA fragment outside the homologous region are 250 to 2000 nucleotides in length.

In certain embodiments, the nucleotide sequences of all of the two or more DNA fragments outside the homologous region are at least 10 nucleotides, 20 nucleotides, 30 nucleotides, 40 nucleotides, 50 nucleotides, 100 nucleotides, 200 nucleotides, 300 nucleotides, 400 nucleotides, 500 nucleotides, 600 nucleotides, 700 nucleotides, 800 nucleotides, 900 nucleotides, 1000 nucleotides, 2000 nucleotides, 5000 nucleotides, 10000 nucleotides, 15000 nucleotides, or 20000 nucleotides, 25000 nucleotides, 30000 nucleotides, 35000 nucleotides, 40000 nucleotides, 45000 nucleotides, or at least 50000 nucleotides in length. In certain embodiments, the nucleotide sequences of all of the two or more DNA fragments outside the homologous region are 10 to 50000 nucleotides in length. In certain embodiments, the nucleotide sequences of the first DNA fragment outside the homologous region are 50 to 10000 nucleotides in length. In certain embodiments, the nucleotide sequences of all of the two or more DNA fragments outside the homologous region are 100 to 5000 nucleotides in length. In certain embodiments, the nucleotide sequences of the second DNA fragment outside the homologous region are 150 to 2500 nucleotides in length. In certain embodiments, the nucleotide sequences of all of the two or more DNA fragments outside the homologous region are 250 to 2000 nucleotides in length.

As used herein and unless otherwise indicated, when two nucleotide sequences have “no homologies” or are “not homologous to” each other, the two nucleotide sequences have at most 1%, 5%, 10%, 15%, 20%, 25%, 30%, 35%, 40%, 45%, 50%, 55%, 60%, 65%, 70%, 75%, or at most 80% sequence identity over a region of about 100 nucleotides, about 125 nucleotides, about 150 nucleotides, about 175 nucleotides, about 200 nucleotides, about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, about 500 nucleotides, about 525 nucleotides, about 550 nucleotides, about 575 nucleotides, about 600 nucleotides, about 625 nucleotides, about 650 nucleotides, about 675 nucleotides, about 700 nucleotides, about 725 nucleotides, about 750 nucleotides, about 775 nucleotides, about 800 nucleotides, about 825 nucleotides, about 850 nucleotides, about 875 nucleotides, about 900 nucleotides, about 925 nucleotides, about 950 nucleotides, about 975 nucleotides, about 1000 nucleotides, about 1025 nucleotides, about 1050 nucleotides, about 1075 nucleotides, or about 2000 nucleotides. In certain embodiments, the two nucleotide sequences may have regions with 90% or higher sequence identity, and such regions are at most about 10 nucleotide, about 20 nucleotide, about 30 nucleotide, or at most about 40 nucleotides in length. In certain embodiments, the two nucleotide sequences may have at most 70% or 80% sequence identity over a region of about 20 nucleotides, about 30 nucleotides, about 40 nucleotides, about 50 nucleotides, about 60 nucleotides, about 70 nucleotides, about 80 nucleotides, about 90 nucleotides, about 100 nucleotides, about 125 nucleotides, about 150 nucleotides, about 175 nucleotides, about 200 nucleotides, about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, or about 500 nucleotides. In certain embodiments, the level of homology in the two nucleotide sequences is not enough to allow homologous recombination of the two nucleotide sequences. In certain embodiments, the level of homology in the two nucleotide sequences may allow at most 1, 2, 3, 4, 5, 6, 7, 8, 9, or 10 undesired recombination events between the two nucleotide sequences per 10,000 copies of nucleotide sequences per 2, 3, 4, 5, 6, 7, 8, 9, 10, 11, 12, 13, 14, or 15 days of incubation.

(i) The First and the Second DNA Fragments are not Homologous to Each Other Outside the 5′ and/or 3′ Homologous Region(s)

In certain embodiments, the nucleotide sequences of the first and the second DNA fragments are not homologous to each other outside the homologous region(s). In certain embodiments, the nucleotide sequences of the first and the second DNA fragments outside the homologous region(s) may have at most 1%, 5%, 10%, 15%, 20%, 25%, 30%, 35%, 40%, 45%, 50%, 55%, 60%, 65%, 70%, 75%, or at most 80% sequence identity over a region of about 100 nucleotides, about 125 nucleotides, about 150 nucleotides, about 175 nucleotides, about 200 nucleotides, about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, about 500 nucleotides, about 525 nucleotides, about 550 nucleotides, about 575 nucleotides, about 600 nucleotides, about 625 nucleotides, about 650 nucleotides, about 675 nucleotides, about 700 nucleotides, about 725 nucleotides, about 750 nucleotides, about 775 nucleotides, about 800 nucleotides, about 825 nucleotides, about 850 nucleotides, about 875 nucleotides, about 900 nucleotides, about 925 nucleotides, about 950 nucleotides, about 975 nucleotides, about 1000 nucleotides, about 1025 nucleotides, about 1050 nucleotides, about 1075 nucleotides, or about 2000 nucleotides. In certain embodiments, the nucleotide sequences of the first and the second DNA fragments outside the homologous region(s) may have regions with 90% or higher sequence identity, and such regions are at most about 10 nucleotide, about 20 nucleotide, about 30 nucleotide, or at most about 40 nucleotides in length. In certain embodiments, the nucleotide sequences of the first and the second DNA fragments outside the homologous region(s) may have at most 70% or 80% sequence identity over a region of about 20 nucleotides, about 30 nucleotides, about 40 nucleotides, about 50 nucleotides, about 60 nucleotides, about 70 nucleotides, about 80 nucleotides, about 90 nucleotides, about 100 nucleotides, about 125 nucleotides, about 150 nucleotides, about 175 nucleotides, about 200 nucleotides, about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, or about 500 nucleotides. In certain embodiments, the level of homology in the nucleotide sequences of the first and the second DNA fragments outside the homologous region is not enough to allow homologous recombination of the DNA fragments in the regions that are outside the homologous region. In certain embodiments, the level of homology in the nucleotide sequences of the first and the second DNA fragments outside the homologous region may allow at most 1, 2, 3, 4, 5, 6, 7, 8, 9, or at most 10 undesired recombination events between the first and the second DNA fragments in the regions that are outside the homologous region per 10,000 copies of each of the first and the second DNA fragments per 2, 3, 4, 5, 6, 7, 8, 9, 10, 11, 12, 13, 14, or 15 days of incubation.

(ii) All DNA Fragments are not Homologous to Each Other Outside the 5′ and/or 3′ Homologous Region(s)

In certain embodiments, the nucleotide sequences of all the DNA fragments are not homologous to each other outside the homologous region(s). In certain embodiments, the nucleotide sequences of all the DNA fragments outside the homologous region(s) may have at most 1%, 5%, 10%, 15%, 20%, 25%, 30%, 35%, 40%, 45%, 50%, 55%, 60%, 65%, 70%, 75%, or at most 80% sequence identity over a region of about 100 nucleotides, about 125 nucleotides, about 150 nucleotides, about 175 nucleotides, about 200 nucleotides, about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, about 500 nucleotides, about 525 nucleotides, about 550 nucleotides, about 575 nucleotides, about 600 nucleotides, about 625 nucleotides, about 650 nucleotides, about 675 nucleotides, about 700 nucleotides, about 725 nucleotides, about 750 nucleotides, about 775 nucleotides, about 800 nucleotides, about 825 nucleotides, about 850 nucleotides, about 875 nucleotides, about 900 nucleotides, about 925 nucleotides, about 950 nucleotides, about 975 nucleotides, about 1000 nucleotides, about 1025 nucleotides, about 1050 nucleotides, about 1075 nucleotides, or about 2000 nucleotides. In certain embodiments, the nucleotide sequences of all the DNA fragments outside the homologous region(s) may have regions with 90% or higher sequence identity, and such regions are at most about 10 nucleotide, about 20 nucleotide, about 30 nucleotide, or at most about 40 nucleotides in length. In certain embodiments, the nucleotide sequences of all the DNA fragments outside the homologous region(s) may have at most 70% or 80% sequence identity over a region of about 20 nucleotides, about 30 nucleotides, about 40 nucleotides, about 50 nucleotides, about 60 nucleotides, about 70 nucleotides, about 80 nucleotides, about 90 nucleotides, about 100 nucleotides, about 125 nucleotides, about 150 nucleotides, about 175 nucleotides, about 200 nucleotides, about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, or about 500 nucleotides. In certain embodiments, the level of homology in the nucleotide sequences of all the DNA fragments outside the homologous region is not enough to allow homologous recombination of the DNA fragments in the regions that are outside the homologous region. In certain embodiments, the level of homology in the nucleotide sequences of all the DNA fragments outside the homologous region may allow at most 1, 2, 3, 4, 5, 6, 7, 8, 9, or at most 10 undesired recombination events of the DNA fragments in the regions that are outside the homologous region per 10,000 copies of each of the DNA fragments per 2, 3, 4, 5, 6, 7, 8, 9, 10, 11, 12, 13, 14, or 15 days of incubation.

(iii) The First and the Second DNA Fragments Outside the 5′ and/or 3′ Homologous Region(s) are not Homologous to a Sequence in the Leishmania Cell's Genome

In certain embodiments, the nucleotide sequences of the first and the second DNA fragments outside the homologous region(s) are not homologous to a sequence in the Leishmania cell's genome. In certain embodiments, the nucleotide sequences of the first and the second DNA fragments outside the homologous region(s) and the Leishmania cell's genome may have at most 1%, 5%, 10%, 15%, 20%, 25%, 30%, 35%, 40%, 45%, 50%, 55%, 60%, 65%, 70%, 75%, or at most 80% sequence identity over a region of about 100 nucleotides, about 125 nucleotides, about 150 nucleotides, about 175 nucleotides, about 200 nucleotides, about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, about 500 nucleotides, about 525 nucleotides, about 550 nucleotides, about 575 nucleotides, about 600 nucleotides, about 625 nucleotides, about 650 nucleotides, about 675 nucleotides, about 700 nucleotides, about 725 nucleotides, about 750 nucleotides, about 775 nucleotides, about 800 nucleotides, about 825 nucleotides, about 850 nucleotides, about 875 nucleotides, about 900 nucleotides, about 925 nucleotides, about 950 nucleotides, about 975 nucleotides, about 1000 nucleotides, about 1025 nucleotides, about 1050 nucleotides, about 1075 nucleotides, or about 2000 nucleotides. In certain embodiments, the nucleotide sequences of the first and the second DNA fragments outside the homologous region(s) and the Leishmania cell's genome may have regions with 90% or higher sequence identity, and such regions are at most about 10 nucleotide, about 20 nucleotide, about 30 nucleotide, or at most about 40 nucleotides in length. In certain embodiments, the nucleotide sequences of the first and the second DNA fragments outside the homologous region(s) and the Leishmania cell's genome may have at most 70% or 80% sequence identity over a region of about 20 nucleotides, about 30 nucleotides, about 40 nucleotides, about 50 nucleotides, about 60 nucleotides, about 70 nucleotides, about 80 nucleotides, about 90 nucleotides, about 100 nucleotides, about 125 nucleotides, about 150 nucleotides, about 175 nucleotides, about 200 nucleotides, about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, or about 500 nucleotides. In certain embodiments, the level of homology in the first and the second DNA fragments outside the homologous region and the Leishmania cell's genome is not enough to allow homologous recombination of the DNA fragments and the Leishmania cell's genome in the regions that are outside the homologous region. In certain embodiments, the level of homology in the first and the second DNA fragments outside the homologous region and the Leishmania cell's genome may allow at most 1, 2, 3, 4, 5, 6, 7, 8, 9, or at most 10 undesired recombination events of the DNA fragments and the Leishmania cell's genome in the regions that are outside the homologous region per 10,000 copies of each of the first and the second DNA fragments per 2, 3, 4, 5, 6, 7, 8, 9, 10, 11, 12, 13, 14, or 15 days of incubation.

(iv) All the DNA Fragments Outside the 5′ and/or 3′ Homologous Region(s) are not Homologous to a Sequence in the Leishmania Cell's Genome

In certain embodiments, the nucleotide sequences of all the DNA fragments outside the homologous region(s) are not homologous to a sequence in the Leishmania cell's genome. In certain embodiments, the nucleotide sequences of the first and the second DNA fragments outside the homologous region(s) and the Leishmania cell's genome may have at most 1%, 5%, 10%, 15%, 20%, 25%, 30%, 35%, 40%, 45%, 50%, 55%, 60%, 65%, 70%, 75%, or at most 80% sequence identity over a region of about 100 nucleotides, about 125 nucleotides, about 150 nucleotides, about 175 nucleotides, about 200 nucleotides, about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, about 500 nucleotides, about 525 nucleotides, about 550 nucleotides, about 575 nucleotides, about 600 nucleotides, about 625 nucleotides, about 650 nucleotides, about 675 nucleotides, about 700 nucleotides, about 725 nucleotides, about 750 nucleotides, about 775 nucleotides, about 800 nucleotides, about 825 nucleotides, about 850 nucleotides, about 875 nucleotides, about 900 nucleotides, about 925 nucleotides, about 950 nucleotides, about 975 nucleotides, about 1000 nucleotides, about 1025 nucleotides, about 1050 nucleotides, about 1075 nucleotides, or about 2000 nucleotides. In certain embodiments, the nucleotide sequences of all the DNA fragments outside the homologous region(s) and the Leishmania cell's genome may have regions with 90% or higher sequence identity, and such regions are at most about 10 nucleotide, about 20 nucleotide, about 30 nucleotide, or at most about 40 nucleotides in length. In certain embodiments, the nucleotide sequences of all the DNA fragments outside the homologous region(s) and the Leishmania cell's genome may have at most 70% or 80% sequence identity over a region of about 20 nucleotides, about 30 nucleotides, about 40 nucleotides, about 50 nucleotides, about 60 nucleotides, about 70 nucleotides, about 80 nucleotides, about 90 nucleotides, about 100 nucleotides, about 125 nucleotides, about 150 nucleotides, about 175 nucleotides, about 200 nucleotides, about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, or about 500 nucleotides. In certain embodiments, the level of homology in the nucleotide sequences of all the DNA fragments outside the homologous region and the Leishmania cell's genome is not enough to allow homologous recombination of the DNA fragments and the Leishmania cell's genome in the regions that are outside the homologous region. In certain embodiments, the level of homology in the nucleotide sequences of all the DNA fragments outside the homologous region and the Leishmania cell's genome may allow at most 1, 2, 3, 4, 5, 6, 7, 8, 9, or at most 10 undesired recombination events of the DNA fragments and the Leishmania cell's genome in the regions that are outside the homologous region per 10,000 copies of each of the DNA fragments per 2, 3, 4, 5, 6, 7, 8, 9, 10, 11, 12, 13, 14, or 15 days of incubation.

(v) The First and the Second DNA Fragments have No Homologies within the Respective DNA Fragment

In certain embodiments, the nucleotide sequences of the first and the second DNA fragments have no homologies within the respective DNA fragment. In certain embodiments, the nucleotide sequences of the first and the second DNA fragments within the respective DNA fragment may contain nucleotide sequences that have at most 1%, 5%, 10%, 15%, 20%, 25%, 30%, 35%, 40%, 45%, 50%, 55%, 60%, 65%, 70%, 75%, or at most 80% sequence identity over a region of about 100 nucleotides, about 125 nucleotides, about 150 nucleotides, about 175 nucleotides, about 200 nucleotides, about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, about 500 nucleotides, about 525 nucleotides, about 550 nucleotides, about 575 nucleotides, about 600 nucleotides, about 625 nucleotides, about 650 nucleotides, about 675 nucleotides, about 700 nucleotides, about 725 nucleotides, about 750 nucleotides, about 775 nucleotides, about 800 nucleotides, about 825 nucleotides, about 850 nucleotides, about 875 nucleotides, about 900 nucleotides, about 925 nucleotides, about 950 nucleotides, about 975 nucleotides, about 1000 nucleotides, about 1025 nucleotides, about 1050 nucleotides, about 1075 nucleotides, or about 2000 nucleotides. In certain embodiments, the nucleotide sequences of the first and the second DNA fragments within the respective DNA fragment may contain nucleotide sequences with 90% or higher sequence identity, and such regions are at most about 10 nucleotide, about 20 nucleotide, about 30 nucleotide, or at most about 40 nucleotides in length. In certain embodiments, the nucleotide sequences of the first and the second DNA fragments within the respective DNA fragment may contain nucleotide sequences that have at most 70% or 80% sequence identity over a region of about 20 nucleotides, about 30 nucleotides, about 40 nucleotides, about 50 nucleotides, about 60 nucleotides, about 70 nucleotides, about 80 nucleotides, about 90 nucleotides, about 100 nucleotides, about 125 nucleotides, about 150 nucleotides, about 175 nucleotides, about 200 nucleotides, about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, or about 500 nucleotides. In certain embodiments, the level of homology in the nucleotide sequences of the first and the second DNA fragments within the respective DNA fragment is not enough to allow homologous recombination of the DNA fragments within itself. In certain embodiments, the level of homology in the nucleotide sequences of the first and the second DNA fragments within the respective DNA fragment may allow at most 1, 2, 3, 4, 5, 6, 7, 8, 9, or at most 10 undesired recombination events within the DNA fragment itself per 10,000 copies of the DNA fragment per 2, 3, 4, 5, 6, 7, 8, 9, 10, 11, 12, 13, 14, or 15 days of incubation.

(vi) All the DNA Fragments have No Homologies within the Respective DNA Fragment

In certain embodiments, the nucleotide sequences of all the DNA fragments have no homologies within the respective DNA fragment. In certain embodiments, the nucleotide sequences of all the DNA fragments within the respective DNA fragment may contain nucleotide sequences that have at most 1%, 5%, 10%, 15%, 20%, 25%, 30%, 35%, 40%, 45%, 50%, 55%, 60%, 65%, 70%, 75%, or at most 80% sequence identity over a region of about 100 nucleotides, about 125 nucleotides, about 150 nucleotides, about 175 nucleotides, about 200 nucleotides, about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, about 500 nucleotides, about 525 nucleotides, about 550 nucleotides, about 575 nucleotides, about 600 nucleotides, about 625 nucleotides, about 650 nucleotides, about 675 nucleotides, about 700 nucleotides, about 725 nucleotides, about 750 nucleotides, about 775 nucleotides, about 800 nucleotides, about 825 nucleotides, about 850 nucleotides, about 875 nucleotides, about 900 nucleotides, about 925 nucleotides, about 950 nucleotides, about 975 nucleotides, about 1000 nucleotides, about 1025 nucleotides, about 1050 nucleotides, about 1075 nucleotides, or about 2000 nucleotides. In certain embodiments, the nucleotide sequences of all the DNA fragments within the respective DNA fragment may contain nucleotide sequences with 90% or higher sequence identity, and such regions are at most about 10 nucleotide, about 20 nucleotide, about 30 nucleotide, or at most about 40 nucleotides in length. In certain embodiments, the nucleotide sequences of all the DNA fragments within the respective DNA fragment may contain nucleotide sequences that have at most 70% or 80% sequence identity over a region of about 20 nucleotides, about 30 nucleotides, about 40 nucleotides, about 50 nucleotides, about 60 nucleotides, about 70 nucleotides, about 80 nucleotides, about 90 nucleotides, about 100 nucleotides, about 125 nucleotides, about 150 nucleotides, about 175 nucleotides, about 200 nucleotides, about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, or about 500 nucleotides. In certain embodiments, the level of homology in the nucleotide sequences of all the fragments within the respective DNA fragment is not enough to allow homologous recombination of the DNA fragments within itself. In certain embodiments, the level of homology in the nucleotide sequences of all the DNA fragments within the respective DNA fragment may allow at most 1, 2, 3, 4, 5, 6, 7, 8, 9, or at most 10 undesired recombination events within the DNA fragment itself per 10,000 copies of the DNA fragment per 2, 3, 4, 5, 6, 7, 8, 9, 10, 11, 12, 13, 14, or 15 days of incubation.

In certain embodiments, the nucleotide sequences of the first and the second DNA fragments outside the homologous region(s) have no repetitive sequences.

In certain embodiments, the number of DNA fragments is at least 2, 3, 4, 5, 6, 7, 8, 9, 10, 11, 12, 13, 14, 15, 16, 17, 18, 19, 20, 21, 22, 23, 24, or at least 25. In certain embodiments, the number of DNA fragments is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 37, 39, 40, 41, 42, 43, 44, 45, 46, 47, 48, 49, or 50.

### 5.1.4 Translation Product of the DNA Fragments

In certain embodiments, the nucleotide sequences of the two or more DNA fragments outside the homologous region are selected from a group consisting of intergenic regions (IRs), untranslated regions (UTRs), and open reading frames (ORFs) encoding polypeptides. In certain embodiments, the nucleotide sequences of the DNA fragments outside the homologous region are selected from a group consisting of intergenic regions (IRs), untranslated regions (UTRs), and open reading frames (ORFs) that are described in the Example section. In certain embodiments, the IRs, UTRs and ORFs are devoid of homologous sequences within itself, and/or homologous sequences to one another.

In certain embodiments, the nucleotide sequences of the DNA fragments outside the homologous region are ORFs that encode target polypeptides as described in Section 5.4. In certain embodiments, the nucleotide sequences of the DNA fragments outside the homologous region are ORFs that encode enzymes related to the production of the target polypeptides. Non-limiting exemplary enzymes may be found in International Publication No. WO2019/002512 A2, incorporated by reference in its entirety herein) and International Application entitled “Glycoengineering Using Leishmania Cells” filed even date herewith. In certain embodiments, the nucleotide sequences of the DNA fragments outside the homologous region are ORFs that encode heterologous glycosyltransferases. In certain embodiments, the nucleotide sequences of the DNA fragments outside the homologous region may be transcribed to RNA products, for example ribozymes, regulating RNA, ncRNA, and crisprRNA). In certain embodiments, the nucleotide sequences of the DNA fragments outside the homologous region may be ORFs that encode polypeptides having the function that relates to catalyzing metabolic reactions and DNA replication, responding to stimuli, transporting molecules from one location to another, providing structure to cells and organisms, aggregation and adhesion to other cells, localization of molecules, utilization of carbon, carbohydrates, nitrogen, phosphorus and sulfur, biomineralization, growth, development and mitosis of cells, locomotion, biological regulation, protein folding, and/or toxins.

In certain embodiments, the nucleotide sequences of the two or more DNA fragments outside the homologous region encode the same polypeptide. In certain embodiments, the Leishmania cell is capable of expressing multiple copies of the same polypeptide. In certain embodiments, the method provided herein increases the expression level of the polypeptide. In certain embodiments, using multiple DNA fragments encoding the same polypeptide may increase the expression level of the polypeptide in comparison to the resulting expression level of the approach using one DNA fragment encoding the polypeptide.
(i) Nucleotide sequence resulted from the homologous recombination of the DNA fragments

In certain embodiments, the homologous recombination of the DNA fragments results in a nucleotide sequence that is 50 nucleotides to 100 nucleotides, 100 nucleotides to 500 nucleotides, 500 nucleotides to 1000 nucleotides, 1000 nucleotides to 5000 nucleotides, 5000 nucleotides to 10000 nucleotides, 10000 nucleotides to 15000 nucleotides, 15000 nucleotides to 20000 nucleotides, 20000 nucleotides to 25000 nucleotides, 25000 nucleotides to 30000 nucleotides, 30000 nucleotides to 35000 nucleotides, 35000 nucleotides to 40000 nucleotides, 40000 nucleotides to 45000 nucleotides, 45000 nucleotides to 50000 nucleotides, 50000 nucleotides to 55000 nucleotides, 55000 nucleotides to 60000 nucleotides, 60000 nucleotides to 65000 nucleotides, 65000 nucleotides to 70000 nucleotides, 70000 nucleotides to 75000 nucleotides, or 75000 nucleotides to 80000 nucleotides in length.

In certain embodiments, the homologous recombination of the DNA fragments results in a nucleotide sequence comprising at least 50%, 60%, 70%, 80%, 90% or 100% of genetic information encoded by the two or more DNA fragments. In certain embodiments, the nucleotide sequence resulted from the homologous recombination of the DNA fragments contains all the genetic information encoded in the two or more DNA fragments.

### 5.1.5 Undesired Crossing Out and/or Crossing Over

In general, the methods provided herein are capable of avoiding undesired genetic recombination events. In certain embodiments, the undesired genetic recombination events include crossing over and crossing out. In certain embodiments, the undesired genetic recombination events may be single-strand annealing (SSA) or micro homology mediated end joining (MMEJ) and non-homologous end joining (NHEJ) (Zhang (2019) Single-Strand Annealing Plays a Major Role in Double-Strand DNA Break Repair following CRISPR-Cas9 Cleavage in Leishmania. doi: 10.1128/mSphere.00408-19.) In certain embodiments, undesired crossing out and/or crossing over may lead to omission of genetic information of the DNA fragments in the nucleotide sequence resulted from the homologous recombination of the DNA fragments. In certain embodiments, undesired crossing out and/or crossing over may lead to omission of genetic information of the chromosomal endogenous DNA.

In certain embodiments, undesired crossing out and/or crossing over may be detected using gene sequencing technologies known in the art. In certain embodiments, undesired crossing out and/or crossing over may be detected by phenotypical testing of the resulting genetically engineered Leishmania cells, for example by testing of the activity of an enzyme that is encoded by one or more DNA fragments used in the method described herein. In certain embodiments, undesired crossing out and/or crossing over may be detected using methods as described in the Assay and Example sections of this application.

In certain embodiments, the method described herein results in low level of undesired crossing out and/or crossing over. In certain embodiments, the undesired crossing out and/or crossing over occurs in at most 0.01%, 0.05%, 0.1%, 0.5%, 1%, 2%, 3%, 4%, 5%, 6%, 7%, 8%, 9% or at most 10% of the Leishmania cells over a period of at least 1 day, 2 days, 3 days, 4 days, 5 days, 6 days, 7 days, 8 days, 9 days, or at least 10 days. In certain embodiments, the undesired crossing out and/or crossing over occurs in about 0.01%, 0.05%, 0.1%, 0.5%, 1%, 2%, 3%, 4%, 5%, 6%, 7%, 8%, 9% or about 10% of the Leishmania cells over a period of at least 1 day, 2 days, 3 days, 4 days, 5 days, 6 days, 7 days, 8 days, 9 days, or at least 10 days.

In certain embodiments, the undesired crossing out and/or crossing over occurs in at most 0.01%, 0.05%, 0.1%, 0.5%, 1%, 2%, 3%, 4%, 5%, 6%, 7%, 8%, 9% or at most 10% of the Leishmania cells over a period of 1 day, 2 days, 3 days, 4 days, 5 days, 6 days, 7 days, 8 days, 9 days, or 10 days. In certain embodiments, the undesired crossing out and/or crossing over occurs in about 0.01%, 0.05%, 0.1%, 0.5%, 1%, 2%, 3%, 4%, 5%, 6%, 7%, 8%, 9% or about 10% of the Leishmania cells over a period of 1 day, 2 days, 3 days, 4 days, 5 days, 6 days, 7 days, 8 days, 9 days, or 10 days.

In certain embodiments, the undesired crossing out and/or crossing over occurs in at most 0.01%, 0.05%, 0.1%, 0.5%, 1%, 2%, 3%, 4%, 5%, 6%, 7%, 8%, 9% or at most 10% of the Leishmania cells over at least 1, 2, 3, 4, 5, 6, 7, 8, 9, or at least 10 cell divisions. In certain embodiments, the undesired crossing out and/or crossing over occurs in about 0.01%, 0.05%, 0.1%, 0.5%, 1%, 2%, 3%, 4%, 5%, 6%, 7%, 8%, 9% or about 10% of the Leishmania cells over at least 1, 2, 3, 4, 5, 6, 7, 8, 9, or at least 10 cell divisions.

In certain embodiments, the undesired crossing out and/or crossing over occurs in at most 0.01%, 0.05%, 0.1%, 0.5%, 1%, 2%, 3%, 4%, 5%, 6%, 7%, 8%, 9% or at most 10% of the Leishmania cells over 1, 2, 3, 4, 5, 6, 7, 8, 9, or 10 cell divisions. In certain embodiments, the undesired crossing out and/or crossing over occurs in about 0.01%, 0.05%, 0.1%, 0.5%, 1%, 2%, 3%, 4%, 5%, 6%, 7%, 8%, 9% or about 10% of the Leishmania cells over 1, 2, 3, 4, 5, 6, 7, 8, 9, or 10 cell divisions.

### 5.1.6 Chromosomal Integration

In certain embodiments, the two or more DNA fragments are suitable for integration in the chromosome of the Leishmania cell.

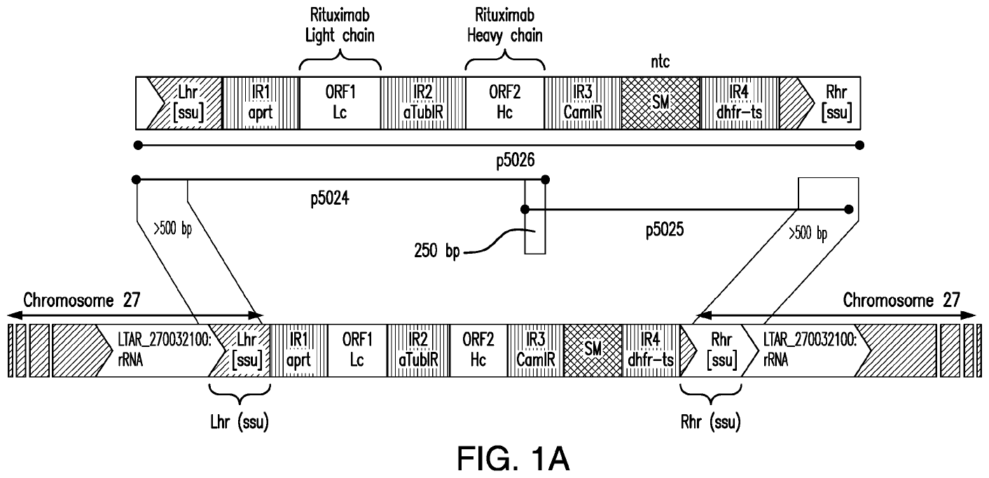

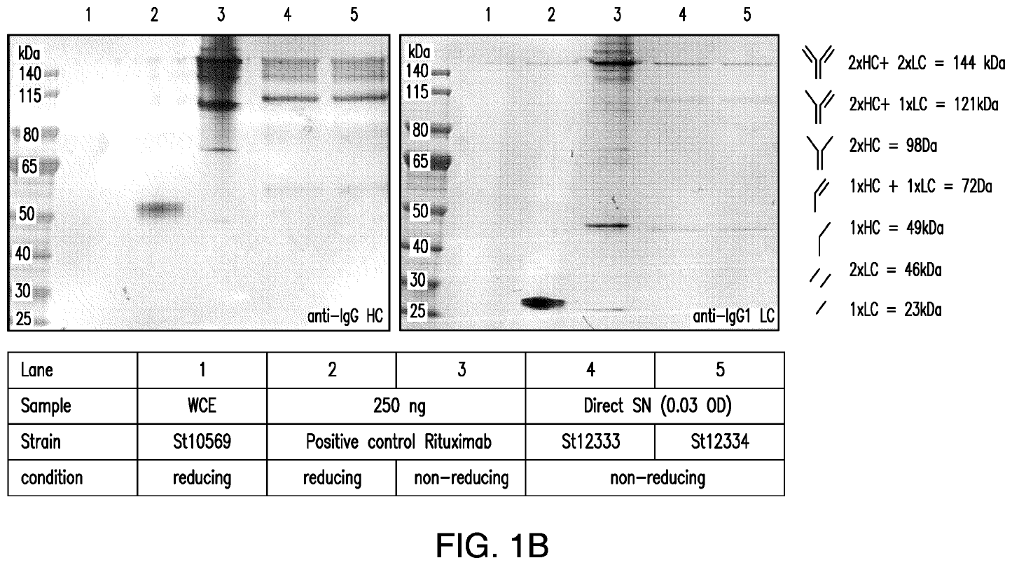

In certain embodiments, the two or more DNA fragments are integrated into the chromosomes of the Leishmania cell. In certain embodiments, one of the DNA fragments comprises a 5′ homologous region that is homologous to a region in the chromosome of the Leishmania cell, and a 3′ homologous region that is homologous to a 5′ homologous region of another DNA fragment. In certain embodiments, one of the DNA fragments comprises a 3′ homologous region that is homologous to another region in the chromosome of the Leishmania cell, and a 5′ homologous region that is homologous to a 3′ homologous region of another DNA fragment. In certain embodiments, the homologous regions that are homologous to regions in the chromosome of the Leishmania cell allow the integration of the DNA fragments into the chromosome of the Leishmania cell. Non-limiting examples of the chromosomal integration described herein may be found in the Example section and illustrated in at least FIGS. 1A, 3B, 4, 6B, and 8A.

### 5.1.7 Extrachromosomal Plasmid

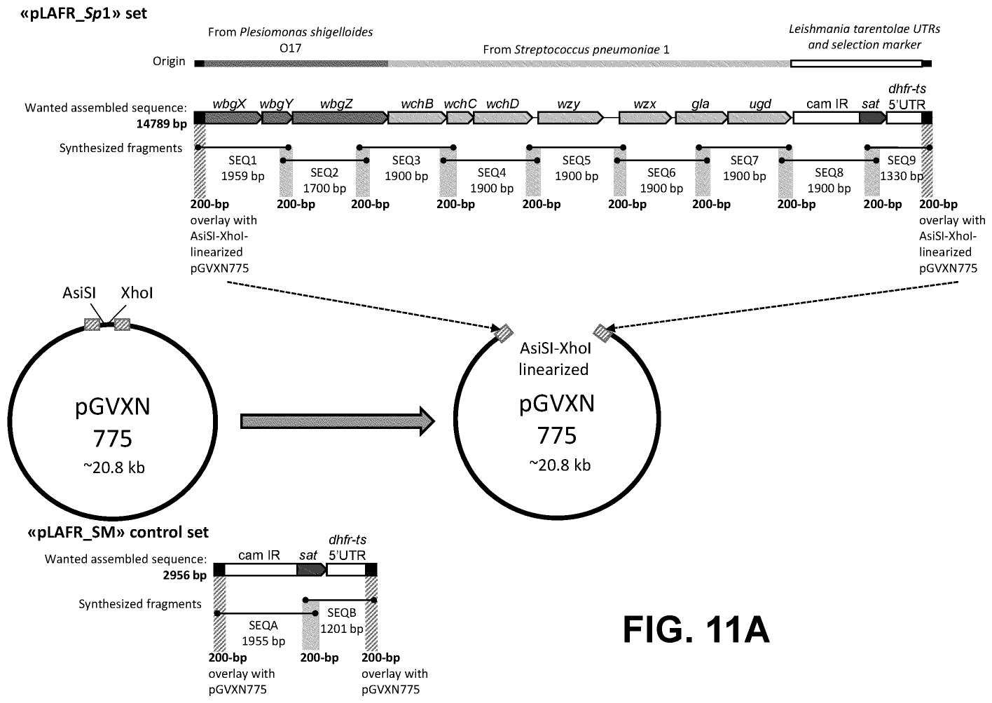

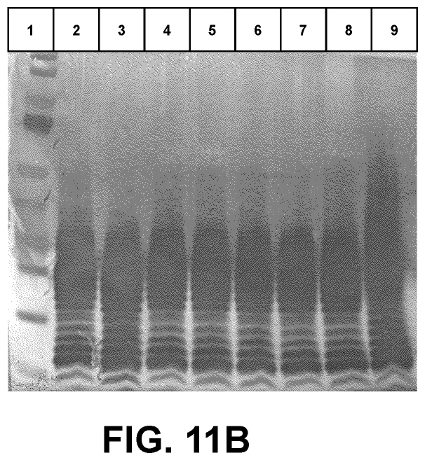

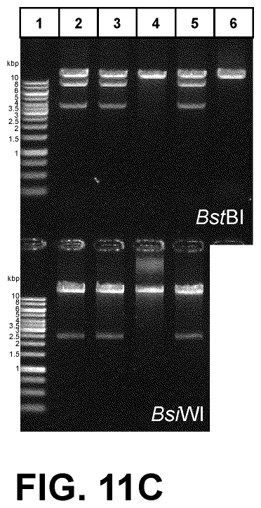

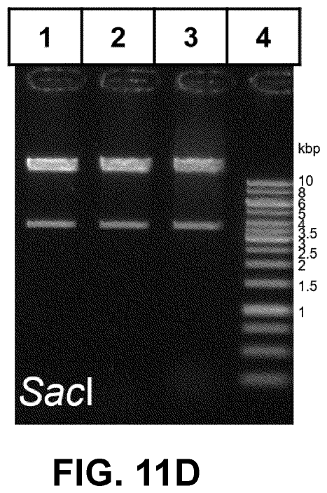

In certain embodiments, the two or more DNA fragments are not integrated in the chromosome of the Leishmania cell. In certain embodiments, the homologous recombination of the two or more DNA fragments results in a circular plasmid. In certain embodiment, the circular plasmid comprises a cos site. In certain embodiment, the circular plasmid is a cosmid. In certain embodiment, the plasmid is an Escherichia coli cosmid. Non-limiting examples of the extrachromosomal plasmid described herein include the Escherichia coli cosmid as described in Example 6 and illustrated in at least FIG. 11A.

### 5.1.8 Introduction of a DNA Fragment into Host Cells

Any method known in the art can be used to introduce a DNA fragment (e.g., a gene fragment thereof) into the host cell, e.g., a Leishmania cell.

In certain embodiments, a DNA fragment is introduced into the host cells described herein using transfection, infection, or electroporation, chemical transformation by heat shock, natural transformation, phage transduction, or conjugation. In a further embodiment, a DNA fragment is introduced into integrated site-specifically into the host cell genome by homologous recombination.

In certain embodiments, a DNA fragment is introduced into the host cells described herein using a plasmid, e.g., a DNA fragment is expressed in the host cells by a plasmid (e.g., an expression vector), and the plasmid is introduced into the modified host cells by transfection, infection, or electroporation, chemical transformation by heat shock, natural transformation, phage transduction, or conjugation. In a specific embodiment, said plasmid is introduced into the modified host cells by stable transfection.

In certain embodiments, the two or more DNA fragments are introduced by transfection. In certain embodiments, the two or more DNA fragments are introduced concurrently.

### 5.1.9 Methods of Culturing Cells

Provided herein are methods for culturing host cells, for example Leishmania host cells. In one embodiment, host cells are cultured using any of the standard culturing techniques known in the art. For example, cells are routinely grown in rich media like Brain Heart Infusion, Trypticase Soy Broth or Yeast Extract, all containing 5 μg/ml Hemin. Additionally, incubation is done at 26° C. in the dark as static or shaking cultures for 2-3 days. In some embodiments, cultures of recombinant cell lines contain the appropriate selective agents. Non-limiting exemplary selective agents are provided in Table 1. In some embodiments, cultures contain Biopterin at a final concentration of 10 μM to support growth. In certain embodiments, host cells may be cultured using the methods as described in the Assay and Examples Sections.

### 5.2 Leishmania Cell

Also provided herein are genetically engineered Leishmania cells. In certain embodiments, the Leishmania cells are genetically engineered using the method described herein in Section 5.1. In certain embodiments, the Leishmania cell is recombinantly engineered using the method described herein repeatedly. In certain embodiments, the Leishmania cells described herein may be used to express the DNA fragments as described in Section 5.1.1. In certain embodiments, the Leishmania cells described herein may be used as an expression system as described in Section 5.3. In certain embodiments, the Leishmania cells described herein may be used to make a polypeptide as described in Section 5.4.

### 5.2.1 Genetically Engineered Leishmania Cells

In certain embodiments, the Leishmania cells are genetically engineered such that they may be used to express the ORFs of the DNA fragments. In certain embodiments, the DNA fragments are integrated into the chromosomes of the Leishmania cell. In certain embodiments, the DNA fragments are not integrated into the chromosomes of the Leishmania cell. In certain embodiments, the homologous recombination of the DNA fragments are circularized to an extrachromosomal plasmid. In certain embodiment, the plasmid is a cosmid. In certain embodiment, the plasmid is an E. coli cosmid.

### 5.2.2 Leishmania and Kinetoplastida Strains

In certain embodiments, the Leishmania cell is a Leishmania tarentolae cell. In certain embodiments, the Leishmania cell is a Leishmania aethiopica cell. In certain embodiments, the Leishmania cell is part of the Leishmania aethiopica species complex. In certain embodiments, the Leishmania cell is a Leishmania aristidesi cell. In certain embodiments, the Leishmania cell is a Leishmania deanei cell. In certain embodiments, the Leishmania cell is part of the Leishmania donovani species complex. In certain embodiments, the Leishmania cell is a Leishmania donovani cell. In certain embodiments, the Leishmania cell is a Leishmania chagasi cell. In certain embodiments, the Leishmania cell is a Leishmania infantum cell. In certain embodiments, the Leishmania cell is a Leishmania hertigi cell. In certain embodiments, the Leishmania cell is part of the Leishmania major species complex. In certain embodiments, the Leishmania cell is a Leishmania major cell. In certain embodiments, the Leishmania cell is a Leishmania martiniquensis cell. In certain embodiments, the Leishmania cell is part of the Leishmania mexicana species complex. In certain embodiments, the Leishmania cell is a Leishmania mexicana cell. In certain embodiments, the Leishmania cell is a Leishmania pifanoi cell. In certain embodiments, the Leishmania cell is part of the Leishmania tropica species complex. In certain embodiments, the Leishmania cell is a Leishmania tropica cell.

In certain embodiments, other host cells may be genetically engineered using the method described herein. In certain embodiments, the host cell belongs to the bodonidae family of kinetoplasts. In a specific embodiment, the host cell is a Bodo saltans cell. In certain embodiments, the host cell belongs to the ichthyobodonidae family of kinetoplasts. In certain embodiments, the host cell belongs to the trypanosomatidae family of kinetoplasts. In certain embodiments, the host cell belongs to the blastocrithidia family of trypanosomatidae. In certain embodiments, the host cell belongs to the blechomonas family of trypanosomatidae. In certain embodiments, the host cell belongs to the herpetomonas family of trypanosomatidae. In certain embodiments, the host cell belongs to the jaenimonas family of trypanosomatidae. In certain embodiments, the host cell belongs to the lafontella family of trypanosomatidae. In certain embodiments, the host cell belongs to the leishmaniinae family of trypanosomatidae. In certain embodiments, the host cell belongs to the novymonas family of trypanosomatidae. In certain embodiments, the host cell belongs to the paratrypanosoma family of trypanosomatidae. In certain embodiments, the host cell belongs to the phytomonas family of trypanosomatidae. In certain embodiments, the host cell belongs to the sergeia family of trypanosomatidae. In certain embodiments, the host cell belongs to the strigomonadinae family of trypanosomatidae. In certain embodiments, the host cell belongs to the trypanosoma family of trypanosomatidae. In certain embodiments, the host cell belongs to the wallacemonas family of trypanosomatidae. In certain embodiments, the host cell belongs to the blastocrithidia family of trypanosomatidae.

### 5.3 Uses of the Leishmania Cell as Expression Systems

In certain embodiments, a Leishmania cell (as described in Section 5.2) may be used as an expression system for making of a polypeptide. In certain embodiments, the polypeptide may be a heterologous, non-Leishmania protein, such as a therapeutic protein (e.g., an antibody).

### 5.3.1 Compositions Comprising Host Cells

In one aspect, provided herein are compositions comprising the host cells described herein, for example, compositions comprising the Leishmania cells as described in Section 5.2. Such compositions can be used in methods for generating a target polypeptide as described in Section 5.4. In certain embodiments, the compositions comprising host cells can be cultured under conditions suitable for the production of polypeptides. Subsequently, the polypeptides can be isolated from said compositions comprising host cells using methods known in the art.

The compositions comprising the host cells provided herein can comprise additional components suitable for maintenance and survival of the host cells described herein, and can additionally comprise additional components required or beneficial to the production of polypeptides by the host cells, e.g., inducers for inducible promoters, such as arabinose, IPTG, tetracycline, and doxycycline.

In certain embodiments, provided herein are kits comprising one or more containers and instructions for use, wherein said one or more containers comprise the Leishmania cell described herein.

### 5.3.2 Methods of Target Polypeptide Production

In one aspect, provided herein are methods of making a target polypeptide as described in Section 5.4. In one embodiment, provided herein is a method of producing a target polypeptide as described in Section 5.4 in vivo, using a host cell described herein. In a specific embodiment, provided herein is a method for producing a target polypeptide, said method comprising (i) culturing a host cell provided herein under conditions suitable for polypeptide production and (ii) isolating said target polypeptide. In a specific embodiment, the host cell comprises (a) a recombinant nucleic acid encoding a target polypeptide; and (b) a recombinant nucleic acid encoding one or more heterologous glycosyltransferases. In certain embodiments, the heterologous glycosyltransferase is an N-acetyl glucosamine transferase; or a heterologous galactosyltransferase; or a heterologous sialyltransferase. In certain embodiments, the host cell is a Leishmania cell.

In one aspect, provided herein are methods of making a polypeptide as described in Section 5.4 comprising (a) culturing the Leishmania cell described herein in Section 5.2 under suitable conditions for polypeptide production; and (b) isolating the polypeptide. In certain embodiments, the method further comprises introducing a nucleotide sequence encoding the polypeptide.

In certain embodiments, the target polypeptide produced by the host cells provided is a therapeutic polypeptide, i.e., a polypeptide used in the treatment of a disease or disorder. For example, the target polypeptide produced by the host cells provided herein can be an enzyme, a cytokine, or an antibody. A list of non-limiting exemplary target polypeptides is provided in Section 5.4.

### 5.4 Target Polypeptide

In one aspect, provided herein are polypeptides produced by the method as described in Section 5.3. In certain embodiments, the target polypeptide produced by the Leishmania cells provided is a therapeutic polypeptide, i.e., a polypeptide used in the treatment of a disease or disorder. For example, the target polypeptide produced by the host cells provided herein can be an enzyme, a cytokine, or an antibody. In certain embodiments, the target the polypeptide is selected from the group consisting of adalimumab, rituximab and erythropoietin (EPO).

Any polypeptide (or peptide/polypeptide corresponding to the polypeptide) known in the art can be used as a target polypeptide in accordance with the methods described herein. One of skill in the art will readily appreciate that the nucleic acid sequence of a known polypeptide, as well as a newly identified polypeptide, can easily be deduced using methods known in the art, and thus it would be well within the capacity of one of skill in the art to introduce a nucleic acid that encodes any polypeptide of interest into a host cell provided herein (e.g., via an expression vector, e.g., a plasmid, e.g., a site specific integration by homologous recombination).

In certain embodiments, the target polypeptide is glycosylated, e.g., sialylated. One of skill in the art will further recognize that the target polypeptides may be glycosylated using the methods described herein, e.g., either in vivo using a host cell provided herein or in vitro, possess therapeutic benefit (e.g., due to improved pharmacokinetics) and thus can be used in the treatment of subjects having diseases/disorders that will benefit from treatment with the glycosylated (e.g., polysialylated) target polypeptides.

In certain embodiments, the target polypeptide comprises the amino acid sequence of human Interferon-α (INF-α), Interferon-β (INF-β), Interferon-γ (INF-γ), Interleukin-2 (IL2), Chimeric diphteria toxin-IL-2 (Denileukin diftitox), Interleukin-1 (IL1), IL1B, IL3, IL4, IL11, IL21, IL22, IL1 receptor antagonist (anakinra), Tumor necrosis factor alpha (TNF-α), Insulin, Pramlintide, Growth hormone (GH), Insulin-like growth factor (IGF1), Human parathyroid hormone, Calcitonin, Glucagon-like peptide-1 agonist (GLP-1), Glucagon, Growth hormone-releasing hormone (GHRH), Secretin, Thyroid stimulating hormone (TSH), Human bone morphogenic polypeptide 2 (hBMP2), Human bone morphogenic proetin 7 (hBMP7), Gonadotropin releasing hormone (GnRH), Keratinocyte growth factor (KGF), Platelet-derived growth factor (PDGF), Fibroblast growth factor 7 (FGF7), Fibroblast growth factor 20 (FGF20), Fibroblast growth factor 21 (FGF21), Epidermal growth factor (EGF), Vascular endothelial growth factor (VEGF), Neurotrophin-3, Human follicle-stimulating hormone (FSH), Human chorionic gonadotropin (HCG), Lutropin-α, Erythropoietin, Granulocyte colony-stimulating factor (G-CSF), Granulocyte-macrophage colony-stimulating factor (GM-CSF), the extracellular domain of CTLA4 (e.g., an FC-fusion), or the extracellular domain of TNF receptor (e.g., an FC-fusion). In a specific embodiment, the target polypeptide used in accordance with the methods and host cells described herein is an enzyme or an inhibitor. Exemplary enzymes and inhibitors that can be used as a target polypeptide include, without limitation, Factor VII, Factor VIII, Factor IX, Factor X, Factor XIII, Factor VIIa, Antithrombin III (AT-III), Polypeptide C, Tissue plasminogen activator (tPA) and tPA variants, Urokinase, Hirudin, Streptokinase, Glucocerebrosidase, Alglucosidase-α, Laronidase (α-L-iduronidase), Idursulphase (Iduronate-2-sulphatase), Galsulphase, Agalsidase-β (human α-galactosidase A), Botulinum toxin, Collagenase, Human DNAse-I, Hyaluronidase, Papain, L-Asparaginase, Uricase (Urate oxidase), glutamate carboxypeptidase (glucarpidase), α1 Protease inhibitor (α1 antitrypsin), Lactase, Pancreatic enzymes (lipase, amylase, protease), and Adenosine deaminase.

In a specific embodiment, the target polypeptide used in accordance with the methods and host cells described herein is a cytokine. Exemplary cytokines that can be used as a target polypeptide include, without limitation, Interferon-α (INF-α), Interferon-β (INF-β), Interferon-γ (INF-γ), Interleukin-2 (IL2), Chimeric diphteria toxin-IL-2 (Denileukin diftitox), Interleukin-1 (IL1), IL1B, IL3, IL4, IL11, IL21, IL22, IL1 receptor antagonist (anakinra), and Tumor necrosis factor alpha (TNF-α).

In a specific embodiment, the target polypeptide used in accordance with the methods and host cells described herein is a hormone or growth factor. Exemplary hormones and growth factors that can be used as a target polypeptide include, without limitation, Insulin, Pramlintide, Growth hormone (GH), Insulin-like growth factor (IGF1), Human parathyroid hormone, Calcitonin, Glucagon-like peptide-1 agonist (GLP-1), Glucagon, Growth hormone-releasing hormone (GHRH), Secretin, Thyroid stimulating hormone (TSH), Human bone morphogenic polypeptide 2 (hBMP2), Human bone morphogenic proetin 7 (hBMP7), Gonadotropin releasing hormone (GnRH), Keratinocyte growth factor (KGF), Platelet-derived growth factor (PDGF), Fibroblast growth factor 7 (FGF7), Fibroblast growth factor 20 (FGF20), Fibroblast growth factor 21 (FGF21), Epidermal growth factor (EGF), Vascular endothelial growth factor (VEGF), Neurotrophin-3, Human follicle-stimulating hormone (FSH), Human chorionic gonadotropin (HCG), Lutropin-α, Erythropoietin, Granulocyte colony-stimulating factor (G-CSF), and Granulocyte-macrophage colony-stimulating factor (GM-CSF).

In a specific embodiment, the target polypeptide used in accordance with the methods and host cells described herein is a receptor. Exemplary receptors that can be used as a target polypeptide include, without limitation, the extracellular domain of human CTLA4 (e.g., fused to an Fc) and the soluble TNF receptor (e.g., fused to an Fc).

In other embodiments, the target polypeptide is a therapeutic polypeptide. In other embodiments, the target polypeptide is an approved biologic drug. In another embodiment, the therapeutic polypeptide comprises the amino acid sequence of Abatacept (e.g., Orencia), Aflibercept (e.g., Eylea), Agalsidase beta (e.g., Fabrazyme), Albiglutide (e.g., Eperzan), Aldesleukin (e.g., Proleukin), Alefacept (e.g., Amevive), Alglucerase (e.g., Ceredase), Alglucosidase alfa (e.g., LUMIZYME), Aliskiren (e.g., Tekturna), Alpha-1-polypeptidease inhibitor (e.g., Aralast), Alteplase (e.g., Activase), Anakinra (e.g., Kineret), Anistreplase (e.g., Eminase), Anthrax immune globulin human (e.g., ANTHRASIL), Antihemophilic Factor (e.g., Advate), Anti-inhibitor coagulant complex (e.g., Feiba Nf), Antithrombin Alfa, Antithrombin III human, Antithymocyte globulin (e.g., Antithymocyte globulin), Anti-thymocyte Globulin (Equine) (e.g., ATGAM), Anti-thymocyte Globulin (Rabbit) (e.g., ATG-Fresenius), Aprotinin (e.g., Trasylol), Asfotase Alfa, Asparaginase (e.g., Elspar), Asparaginase Erwinia chrysanthemi (e.g., Erwinaze), Becaplermin (e.g., REGRANEX), Belatacept (e.g., Nulojix), Beractant, Bivalirudin (e.g., Angiomax), Botulinum Toxin Type A (e.g., BOTOXE), Botulinum Toxin Type B (e.g., Myobloc), Brentuximab vedotin (e.g., Adcetris), Buserelin (e.g., Suprecur), Cl Esterase Inhibitor (Human), Cl Esterase Inhibitor (Recombinant) (e.g., Ruconest), Certolizumab pegol (e.g., Cimzia), Choriogonadotropin alfa (e.g., Choriogonadotropin alfa), Chorionic Gonadotropin (Human) (e.g., Ovidrel), Chorionic Gonadotropin (Recombinant) (e.g., Ovitrelle), Coagulation factor ix (e.g., Alprolix), Coagulation factor VIIa (e.g., NovoSeven), Coagulation factor X human (e.g., Coagadex), Coagulation Factor XIII A-Subunit (Recombinant), Collagenase (e.g., Cordase), Conestat alfa, Corticotropin (e.g., H.P. Acthar), Cosyntropin (e.g., Cortrosyn), Darbepoetin alfa (e.g., Aranesp), Defibrotide (e.g., Noravid), Denileukin diftitox (e.g., Ontak), Desirudin, Digoxin Immune Fab (Ovine) (e.g., DIGIBIND), Dornase alfa (e.g., Pulmozyme), Drotrecogin alfa (e.g., Xigris), Dulaglutide, Efmoroctocog alfa (e.g., ELOCTA), Elosulfase alfa, Enfuvirtide (e.g., FUZEON), Epoetin alfa (e.g., Binocrit), Epoetin zeta (e.g., Retacrit), Eptifibatide (e.g., INTEGRILIN), Etanercept (e.g., Enbrel), Exenatide (e.g., Byetta), Factor IX Complex (Human) (e.g., AlphaNine), Fibrinolysin aka plasmin (e.g., Elase), Filgrastim (e.g., N.A.), Filgrastim-sndz, Follitropin alfa (e.g., Gonal-F), Follitropin beta (e.g., Follistim AQ), Galsulfase (e.g., Naglazyme), Gastric intrinsic factor, Gemtuzumab ozogamicin (e.g., Mylotarg), Glatiramer acetate (e.g., Copaxone), Glucagon recombinant (e.g., GlucaGen), Glucarpidase (e.g., Voraxaze), Gramicidin D (e.g., Neosporin), Hepatitis B immune globulin, Human calcitonin, Human Clostridium tetani toxoid immune globulin, Human rabies virus immune globulin (e.g., Hyperab Rabies Immune Globulin Human), Human Rho(D) immune globulin (e.g., Hyp Rho D Inj 16.5%), Human Serum Albumin (e.g., Albuminar), Human Varicella-Zoster Immune Globulin (e.g., Varizig), Hyaluronidase (e.g., HYLENEX), Hyaluronidase (Human Recombinant), Ibritumomab tiuxetan (e.g., Zevalin), Idursulfase (e.g., Elaprase), Imiglucerase (e.g., Cerezyme), Immune Globulin Human, Insulin aspart (e.g., NovoLog), Insulin Beef, Insulin Degludec (e.g., Tresiba), Insulin detemir (e.g., LEVEMIR), Insulin Glargine (e.g., Lantus), Insulin glulisine (e.g., APIDRA), Insulin Lispro (e.g., Humalog), Insulin Pork (e.g., Iletin II), Insulin Regular (e.g., Humulin R), Insulin, porcine (e.g., vetsulin), Insulin, isophane (e.g., Novolin N), Interferon Alfa-2a, Recombinant (e.g., Roferon A), Interferon alfa-2b (e.g., INTRON A), Interferon alfacon-1 (e.g., INFERGEN), Interferon alfa-n1 (e.g., Wellferon), Interferon alfa-n3 (e.g., Alferon), Interferon beta-1a (e.g., Avonex), Interferon beta-1b (e.g., Betaseron), Interferon gamma-1b (e.g., Actimmune), Intravenous Immunoglobulin (e.g., Civacir), Laronidase (e.g., Aldurazyme), Lenograstim (e.g., Granocyte), Lepirudin (e.g., Refludan), Leuprolide (e.g., Eligard), Liraglutide (e.g., Saxenda), Lucinactant (e.g., Surfaxin), Lutropin alfa (e.g., Luveris), Mecasermin (e.g., N.A.), Menotropins (e.g., Menopur), Methoxy polyethylene glycol-epoetin beta (e.g., Mircera), Metreleptin (e.g., Myalept), Natural alpha interferon OR multiferon (e.g., Intron/Roferon-A), Nesiritide (e.g., NATRECOR), Ocriplasmin (e.g., Jetrea), Oprelvekin (e.g., Neumega), OspA lipopolypeptide (e.g., Lymerix), Oxytocin (e.g., Pitocin), Palifermin (e.g., Kepivance), Pancrelipase (e.g., Pancrecarb), Pegademase bovine (e.g., Adagen), Pegaspargase (e.g., Oncaspar), Pegfilgrastim (e.g., Neulasta), Peginterferon alfa-2a (e.g., Pegasys), Peginterferon alfa-2b (e.g., PEG-Intron), Peginterferon beta-1a (e.g., Plegridy), Pegloticase (e.g., (Krystexxa)), Pegvisomant (e.g., SOMAVERT), Poractant alfa (e.g., Curosurf), Pramlintide (e.g., Symlin), Preotact (e.g., PreotactE), Protamine sulfate (e.g., Protamine Sulfate Injection, USP), Polypeptide S human (e.g., Polypeptide S human), Prothrombin (e.g., Feiba Nf), Prothrombin complex (e.g., Cofact), Prothrombin complex concentrate (e.g., Kcentra), Rasburicase (e.g., Elitek), Reteplase (e.g., Retavase), Rilonacept (e.g., Arcalyst), Romiplostim (e.g., Nplate), Sacrosidase (e.g., Sucraid), Salmon Calcitonin (e.g., Calcimar), Sargramostim (e.g., Leucomax), Satumomab Pendetide (e.g., OncoScint), Sebelipase alfa (e.g., Kanuma), Secretin (e.g., SecreFlo), Sermorelin (e.g., Sermorelin acetate), Serum albumin (e.g., Albunex), Serum albumin iodonated (e.g., Megatope), Simoctocog Alfa (e.g., Nuwiq), Sipuleucel-T (e.g., Provenge), Somatotropin Recombinant (e.g., NutropinAQ), Somatropin recombinant (e.g., BioTropin), Streptokinase (e.g., Streptase), Susoctocog alfa (e.g., Obizur), Taliglucerase alfa (e.g., Elelyso), Teduglutide (e.g., Gattex), Tenecteplase (e.g., TNKase), Teriparatide (e.g., Forteo), Tesamorelin (e.g., Egrifta), Thrombomodulin Alfa (e.g., Recomodulin), Thymalfasin (e.g., Zadaxin), Thyroglobulin, Thyrotropin Alfa (e.g., Thyrogen), Tuberculin Purified Polypeptide Derivative (e.g., Aplisol), Turoctocog alfa (e.g., Zonovate), Urofollitropin (e.g., BRAVELLE), Urokinase (e.g., Kinlytic), Vasopressin (e.g., Pitressin), Velaglucerase alfa (e.g., Vpriv), Abciximab (e.g., ReoPro), Adalimumab (e.g., Humira), Alemtuzumab (e.g., CAMPATH), Alirocumab (e.g., Praluent), Arcitumomab (e.g., CEA-Scan), Atezolizumab (e.g., Tecentriq), Basiliximab (e.g., Simulect), Belimumab (e.g., Benlysta), Bevacizumab (e.g., Avastin), Blinatumomab (e.g., Blincyto), Brodalumab (e.g., Siliq), Canakinumab (e.g., ILARISE), Canakinumab (e.g., Ilaris), Capromab (e.g., ProstaScint), Cetuximab (e.g., Erbitux), Daclizumab (e.g., Zenapax), Daratumumab (e.g., DARZALEX), Denosumab (e.g., Xgeva), Dinutuximab (e.g., unituxin), Eculizumab (e.g., Soliris), Efalizumab (e.g., RAPTIVA), Elotuzumab (e.g., EMPLICITI), Evolocumab (e.g., Repatha), Golimumab (e.g., Simponi Injection), Ibritumomab (e.g., Zevalin), Idarucizumab (e.g., Praxbind), Infliximab (e.g., REMICADE), Ipilimumab (e.g., YERVOY), Ixekizumab (e.g., Taltz), Mepolizumab (e.g., Nucala), Muromonab (e.g., ORTHOCLONE OKT3), Natalizumab (e.g., Tysabri), Necitumumab (e.g., Portrazza), Nivolumab (e.g., Opdivo), Obiltoxaximab (e.g., Anthim), Obinutuzumab (e.g., Gazyva), Ofatumumab (e.g., Arzerra), Omalizumab (e.g., Xolair), Palivizumab (e.g., Synagis), Panitumumab (e.g., Vectibix), Pembrolizumab (e.g., Keytruda), Pertuzumab (e.g., Perjeta), Ramucirumab (e.g., Cyramza), Ranibizumab (e.g., Lucentis), Raxibacumab (e.g., RAXIBACUMAB), Rituximab (e.g., Rituxan), Secukinumab (e.g., Cosentyx), Siltuximab (e.g., Sylvant), Tocilizumab (e.g., ACTEMRA), Tositumomab (e.g., Bexxar), Trastuzumab (e.g., Herceptin), Ustekinumab (e.g., Stelara), or Vedolizumab (e.g., Entyvio).

In other embodiments, the target polypeptide is an antibody. In further embodiments, the antibody has the amino acid sequence of adalimumab (Humira); Remicade (Infliximab); ReoPro (Abciximab); Rituxan (Rituximab); Simulect (Basiliximab); Synagis (Palivizumab); Herceptin (Trastuzumab); Mylotarg (Gemtuzumab ozogamicin); Campath (Alemtuzumab); Zevalin (Ibritumomab tiuxetan); Xolair (Omalizumab); Bexxar (Tositumomab-I-131); Erbitux (Cetuximab); Avastin (Bevacizumab); Tysabri (Natalizumab); Actemra (Tocilizumab); Vectibix (Panitumumab); Lucentis (Ranibizumab); Soliris (Eculizumab); Cimzia (Certolizumab pegol); Simponi (Golimumab); Ilaris (Canakinumab); Stelara (Ustekinumab); Arzerra (Ofatumumab); Prolia (Denosumab); Numax (Motavizumab); ABThrax (Raxibacumab); Benlysta (Belimumab); Yervoy (Ipilimumab); Adcetris (Brentuximab Vedotin); Perjeta (Pertuzumab); Kadcyla (Ado-trastuzumab emtansine); or Gazyva (Obinutuzumab).

In other embodiments, the antibody is a full length antibody, an Fab, an F(ab′)2, an Scfv, or a sdAb. In other embodiments, the target polypeptide comprises the amino acid sequence of an enzyme or an inhibitor thereof. In another embodiment, the target polypeptide comprises the amino acid sequence of Factor VII, Factor VIII, Factor IX, Factor X, Factor XIII, Factor VIIa, Antithrombin III (AT-III), Polypeptide C, Tissue plasminogen activator (tPA) and tPA variants, Urokinase, Hirudin, Streptokinase, Glucocerebrosidase, Alglucosidase-α, Laronidase (α-L-iduronidase), Idursulphase (Iduronate-2-sulphatase), Galsulphase, Agalsidase-β (human α-galactosidase A), Botulinum toxin, Collagenase, Human DNAse-I, Hyaluronidase, Papain, L-Asparaginase, Uricase (Urate oxidase), glutamate carboxypeptidase (glucarpidase), al Protease inhibitor (α1 antitrypsin), Lactase, Pancreatic enzymes (lipase, amylase, protease), and Adenosine deaminase.

In a specific embodiment, the target polypeptide used in accordance with the methods and host cells described herein is a receptor. Exemplary receptors that can be used as a target polypeptide include, without limitation, the extracellular domain of human CTLA4 (e.g., fused to an Fc) and the soluble TNF receptor (e.g., fused to an Fc).

In another embodiment, the target polypeptide is secreted into the culture media. In certain embodiments, the target polypeptide is purified from the culture media. In another embodiment, the target polypeptide is purified from the culture media via affinity purification or ion exchange chromatography. In another embodiment, the target polypeptide contains an Fc domain and is affinity purified from the culture media via polypeptide-A. In another embodiment, the target polypeptide contains an affinity tag and is affinity purified.

In certain embodiments, the target polypeptide used in accordance with the methods and host cells described herein can be a full length polypeptide, a truncation, a polypeptide domain, a region, a motif or a peptide thereof.

In certain embodiments, the target polypeptide is an Fc-fusion polypeptide.

In certain embodiments, the target polypeptide is a biologic comprising an Fc domain of an IgG.

In certain embodiment, the target polypeptide could be modified. In another embodiment, the target polypeptide has been engineered to comprise a signal sequence from Leishmania. In other embodiments, the signal sequence is processed and removed from the target polypeptide. In another embodiment, the target polypeptide has been engineered to comprise one or more tag(s). In other embodiments, the tag is processed and removed from the target polypeptide.

### 5.4.1 Composition and/or Formulation Comprising the Polypeptide

In another aspect, provided herein are compositions (e.g., pharmaceutical compositions) comprising one or more of the target polypeptides described herein. The compositions described herein are useful in the treatment and/or prevention of diseases/disorders in subjects (e.g., human subjects) (see Section 5.4.2).

In certain embodiments, in addition to comprising a target polypeptide described herein, the compositions (e.g., pharmaceutical compositions) described herein comprise a pharmaceutically acceptable carrier. As used herein, the term “pharmaceutically acceptable” means approved by a regulatory agency of the Federal or a state government or listed in the U.S. Pharmacopeia or other generally recognized pharmacopeiae for use in animals, and more particularly in humans. The term “carrier,” as used herein in the context of a pharmaceutically acceptable carrier, refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin.

In certain embodiments, the compositions described herein are formulated to be suitable for the intended route of administration to a subject. For example, the compositions described herein may be formulated to be suitable for subcutaneous, parenteral, oral, intradermal, transdermal, colorectal, intraperitoneal, and rectal administration. In a specific embodiment, the pharmaceutical composition may be formulated for intravenous, oral, intraperitoneal, intranasal, intratracheal, subcutaneous, intramuscular, topical, intradermal, transdermal or pulmonary administration.

In certain embodiments, the compositions described herein additionally comprise one or more buffers, e.g., phosphate buffer and sucrose phosphate glutamate buffer. In other embodiments, the compositions described herein do not comprise buffers.

In certain embodiments, the compositions described herein additionally comprise one or more salts, e.g., sodium chloride, calcium chloride, sodium phosphate, monosodium glutamate, and aluminum salts (e.g., aluminum hydroxide, aluminum phosphate, alum (potassium aluminum sulfate), or a mixture of such aluminum salts). In other embodiments, the compositions described herein do not comprise salts.

The compositions described herein can be included in a kit, container, pack, or dispenser together with instructions for administration.

The compositions described herein can be stored before use, e.g., the compositions can be stored frozen (e.g., at about −20° C. or at about −70° C.); stored in refrigerated conditions (e.g., at about 4° C.); or stored at room temperature.

### 5.4.2 Prophylactic and Therapeutic Uses

In one aspect, provided herein are methods of preventing or treating a disease or disorder in a subject comprising administering to the subject a target polypeptide described herein or a composition thereof. Further provided herein are methods of preventing a disease or disorder in a subject comprising administering to the subject a target polypeptide described herein or a composition thereof.

In one aspect, provided herein are methods of treating a disease or disorder in a subject comprising administering to the subject a target polypeptide described herein or a composition thereof. In another aspect, provided herein are methods of preventing a disease or disorder in a subject comprising administering to the subject a target polypeptide described herein or a composition thereof. In a specific embodiment, provided herein is a method for treating or preventing a disease or disorder in a subject comprising administering to the subject a polysialylated target polypeptide produced according to the methods described herein.

In certain embodiments, the disease or disorder may be caused by the presence of a defective version of a target polypeptide in a subject, the absence of a target polypeptide in a subject, diminished expression of a target polypeptide in a subject can be treated or prevented using the target polypeptides produced using the methods described herein. In certain embodiments, the diseases or disorder may be mediated by a receptor that is bound by a target polypeptide produced using the methods described herein, or mediated by a ligand that is bound by a target polypeptide produced using the methods described herein (e.g., where the target polypeptide is a receptor for the ligand).

In certain embodiments, the methods of preventing or treating a disease or disorder in a subject comprise administering to the subject an effective amount of a target polypeptide described herein or a composition thereof. In certain embodiments, the effective amount is the amount of a therapy which has a prophylactic and/or therapeutic effect(s). In certain embodiments, an “effective amount” refers to the amount of a therapy which is sufficient to achieve one, two, three, four, or more of the following effects: (i) reduce or ameliorate the severity of a disease/disorder or symptom associated therewith; (ii) reduce the duration of a disease/disorder or symptom associated therewith; (iii) prevent the progression of a disease/disorder or symptom associated therewith; (iv) cause regression of a disease/disorder or symptom associated therewith; (v) prevent the development or onset of a disease/disorder, or symptom associated therewith; (vi) prevent the recurrence of a disease/disorder or symptom associated therewith; (vii) reduce organ failure associated with a disease/disorder; (viii) reduce hospitalization of a subject having a disease/disorder; (ix) reduce hospitalization length of a subject having a disease/disorder; (x) increase the survival of a subject with a disease/disorder; (xi) eliminate a disease/disorder in a subject; and/or (xii) enhance or improve the prophylactic or therapeutic effect(s) of another therapy.

### 5.5 Assay

### 5.5.1 Strains, Growth and Genetic Methods

Provided herein are methods for culturing host cells.

Host cells are cultured using any of the standard culturing techniques known in the art. For example, cells are routinely grown in rich media like Brain Heart Infusion, Trypticase Soy Broth or Yeast Extract, all containing 5 μg/ml Hemin. Additionally, incubation is done at 26° C. in the dark as static or shaking cultures for 2-3d. In some embodiments, cultures of recombinant cell lines contain the appropriate selective agents.

A non-limiting list of selective agents is provided in Table 1.

TABLE 1
Selective agents used during transfection (50%
concentration for preselection and
100% concentration for main selection) and standard
culturing of L. tarentolae. Double amounts of the selective
agents could be used if higher selection pressure was intended.

| | Resistance | Concentration (100%) | Concentration |
|---|---|---|---|
| | conferring | main selection/standard | (50%) |
| Selective agent | gene | culturing | preselection |
| Nourseothricin | sat | 50 μg/ml | 25 μg/ml |
| Geneticin | neo | 50 μg/ml | 25 μg/ml |
| Paromomycin | neo | 300 μg/ml | 150 μg/ml |
| Zeocin | ble | 150 μg/ml | 75 μg/ml |
| Hygromycin | hyg | 50 μg/ml | 25 μg/ml |
| Blasticidin | bsd | 5 μg/ml | 2.5 μg/ml |
| Puromycin | pac | 5 μg/ml | 2.5 μg/ml |

### 5.5.2 Plasmids

Plasmids were derived from a pUC57 vector backbone for E. coli propagation and contained an ampicillin or kanamycin section marker. The expression cassettes are flanked by restriction sites suitable for excision. The composition of the cassettes depends on the intended use and is described in the respective methods and examples. The genes of interest are included as ORFs that were codon usage optimized for L. tarentolae by backtranslation of the protein sequences to nucleotide sequences using a custom Python3 script that stochastically selects codons based on the L. tarentolae codon usage frequency while excluding rare codons (frequency <10%). The codon usage has been calculated using cusp (Rice, et al. (2000) Trends in genetics: TIG 16 (6), pp. 276-277) on all annotated L. tarentolae nucleotide coding sequences. Optimized sequences were manually curated for avoidance of restriction sites and deletion of repeats or homopolymer stretches.

To select new intergenic regions for the generation of artificial polycistrons, the genomes of L. mexicana, L. donovani, and L. infantum were searched for homologs to L. major genes that were shown to have high relative expression transcript levels (Rastrojo, et al. (2013) BMC Genomics 14, p. 223) and the associated 3′ intergenic regions (Murray, et al. (2007) Molecular and Biochemical Parasitology 153 (2), pp. 125-132) were further filtered using blastn (Camacho, et al. (2009) BMC Bioinformatics 10, p. 421) to exclude those that have more than 80% identity to each other (using cd-hit (Li, et al. (2006) Bioinformatics 22 (13), pp. 1658-1659)) or identical stretches of more than 30 bp to the L. tarentolae genome.

For long integration constructs, the constructs were split into several pieces of usually less than 2500 bp that contained regions for homologous recombination with either other fragments (usually 200 bp) or the chromosomal integration locus (usually 500 bp) in their extremities to allow assembly by the Leishmania tarentolae homologous recombination system.

The plasmids were generated and sequenced by a gene synthesis provider. Plasmids and descriptions are found in the sequence listings.

(i) Transfection Method

(A) Preparation of DNA

Restriction digest (12 μg DNA in total volume of 240 μL) was performed using standard restriction enzymes (ThermoFisher, preferably FastDigest) according to the manufacturer's instructions. The restriction digest was performed until completion or o/n at 30° C. and purified DNA by EtOH precipitation (2 volume 100% ice cold EtOH was added to 1 volume digested DNA, incubated 30 min on ice, centrifuged for 30 min 17′500×g at 4° C. Pellet was washed with 70% EtOH and subsequently dried for maximum 15 min before resuspension in ddH₂O. For optimized removal of circularized plasmid, 1 or 2 restriction enzymes with recognition sites in the vector backbones were chosen and a digest was done for 1 h at 37° C. and purified by EtOH as described above. The digest was analyzed by agarose gel electrophoresis in 0.7-2% agarose gels (TAE buffer). Optionally, gel extraction was performed with the NucleoSpin® Gel and PCR Clean-up kit (Macherey&Nagel) according to manufacturer's instructions to remove undigested plasmid from the preparation.

(B) DNA Preparation for Transfection

The linear DNA fragments for integration are mixed for transfection in the needed combinations at 1 μg per fragment. The volume of the mix was reduced to approximately 2 μl per transfection in a vacuum concentrator at 30° C. For episomal transfection of plasmids, 0.1-1 of plasmid DNA were directly used for transfection.

(C) Transfection with Nucleofector

One day before transfection, a densely grown culture of the parental strain was diluted 1:10 into fresh media (Brain Heart Infusion plus Hemin, “BHIH”; or Yeast Extract plus Hemin, “YEH”) containing all antibiotics for which selection markers were previously integrated and cultured overnight at 26° C.

Transfection was performed using the 4DNucleofector™_Core_X with the P3 Primary Cell 4D-Nucleofector™ X Kit (Lonza). For this, DNA as prepared above was mixed with 16.4 μl P3 Primary Cell solution and 3.6 μl Supplement Solution. The equivalent culture volume of 10⁷ cells (OD should be around 0.3-1.0/ml, cell shape round to drop-like) was pelleted by centrifugation at 1800 g for 5 min and the supernatant was removed. The cell pellet was resuspended in the DNA mix and transfected using a 16-well electroporation strip with pulse FI-158 (in some examples alternative pulses FP167, CM150, EO115, DN100, FP158, FB158 were used). As negative control, an additional culture was transfected with ddH₂O only.

80 μl of fresh media (BHIH or YEH plus parental cell line selection markers) was added to each well and 2×45 μl of the mix were transferred to individual wells of a 96 well culture plate that were prefilled with 200 μl of fresh medium. After incubation for 24 h at 26° C. in the dark (recovery), the new selection marker was added at 50% concentration (preselection; see table 2). After further incubation for 1-2 days, the selection marker was topped up to 100% (main selection, see table) and several dilutions between 1:2 and 1:10 were performed in 96 well format (final volume 250 μl). Cultures were further incubated at 26° C. in the dark for up to 7 days. If no growth was observed, the culture medium was replaced (centrifugation at 1800 g, 10 min, RT) and cultures were again incubated for up to 7 days. This step was repeated if necessary. Growing cultures were expanded in to higher culture volumes by dilutions in the range of 1:5 and 1:20 before analysis.

(D) Transfection with Gene Pulser Xcell™ (Biorad)

Preparation of the Leishmania culture for transfection was done by a 1:10 dilution of a densely grown culture in BHIH or YEH the day before transfection, static at 26° C. The OD was measured at 600 nm with photometer in single-use cuvettes and ranged be between 0.4-1.0 (4-6×10*7 cells) for optimal efficiency. The cells should be in log-phase, which is indicated by a mixed population out of round and drop-like shaped cells. More round shaped cells were preferred. 10 ml culture was used for one transfection and one culture was always electroporated with ddH₂O as negative control for the respective selection marker. For transfection the culture was spun at 1 ‘800×g for 5 min, RT. The SN was removed and pellet resuspended in 5 ml transfection buffer (200 mM Hepes pH 7.0, 137 mM NaCl, 5 mM KCl, 0.7 mM Na2HPO4, 6 mM dextrose, anhydrous (glucose), sterile filtered 0.22 um). Cells were centrifuged again and the pellet was resuspended in 400 μl transfection buffer. 400 μl of cells were added to the DNA and transferred into the cuvettes and incubate on ice for 10 min. Electroporation was performed with a Gene Pulser Xcell™ (Biorad) using a low voltage protocol (μ exp. decay: 450 V, 450 μF, 5-6 ms, cuvette: d=2 mm) and immediately put on ice for exact 10 min. The whole content of cuvette was transferred into 10 ml BHIH or YEH without any selection marker and cells were grown at 26° C. in dark, aerated, static for 20-24 h. For the selection of a polyclonal cell line, half concentration of selection marker was added and cultures were incubated at 26° C. for 1-2 days and then passaged 1:10 in 10 ml BHIH or YEH with full concentration of selection marker. Cells were grown further at 26° C. in dark. If after 7 days cultures were turning into turbid culture, cells would be spun down at 1′800×g for 5 min at RT and pellet is resuspended in new BHIH or YEH media containing full selection marker concentration.

(ii) Clonal Selection

For clonal selection, cells were streaked on BHIH or YEH plates (containing 1.4% agar and the appropriate 100% selective agent) as soon as the liquid culture turned turbid. Plates were sealed with parafilm and incubated 7-10 days upside down in dark at 26° C. Single colonies (1-2 mm size) were transferred into 24-well plates containing 1 ml BHIH or YEH, sealed with parafilm and incubated in dark at 26° C. for around 7-10 days. 1 ml culture was then transferred from 24-well plate into 10 ml BHI or YEH in a flask and further grown statically as usual.

(iii) Extraction of Genomic DNA by Gravity Flow Method for Long Read Sequencing

Genomic DNA was extracted from 10 ml of dense Leishmania tarentolae culture (grown for 3 days; OD approx. 2) by using the Macherey Nagel NucleoBond CB 100 Kit #740508 (Nucleobond Buffer set IV #740604 with AXG 100 columns). For this, the cells were pelleted for 15 min at 1600 g and washed twice with 10 ml PBS. Next, the cell pellet was resuspended in 1 ml PBS and subjected to the extraction protocol according to manufacturer's instructions.

(iv) Nanopore Sequencing

Construction of strains St16834, St17311, St17212, St17180 were verified by long read Nanopore sequencing. Library preparation was performed according to the manufacturer's instructions (Oxford Nanopore Technologies, Oxford, UK). Nanopore sequencing was performed on a GridION X5 instrument (Oxford Nanopore Technologies) with real-time base calling enabled. Sequencing runs were terminated after 48 h. Raw reads were assembled using Canu hierarchical assembler (version 1.8) (Koren, et al. (2017) Genome research 27 (5), pp. 722-736). Assembled contigs were compared to the target in silico reference sequence using BLAST (Camacho, et al. (2009) BMC Bioinformatics 10, p. 421) and Artemis Comparison Tool (Carver, et al. (2005 Bioinformatics 21 (16), pp. 3422-3423).

(v) PacBio Sequencing

PacBio long read genome sequencing was performed on 2 PacBio SMRT cell (v2.1 chemistry) for St15448 and 1 PacBio sequel SMRT cell for St17527 with the library preparation according to the manufacturer's specification).

PacBio raw reads were assembled into long contigs using HGAP [https://github.com/PacificBiosciences/Bioinformatics-Training/wiki/HGAP-in-SIVIRT-Analysis] and error corrected using two rounds of Arrow [https://github.com/PacificBiosciences/GenomicConsensus].

(vi) Illumina Sequencing

Genomic DNA of St17527 was additionally sequenced on Illumina NextSeq (2×150 bp paired-end sequencing; TruSeq library preparation according to the manufacturer's specification). The resulting quality trimmed data consists of approximately 20M paired reads per strain. BWA-MEM (Li, Heng (2013) Aligning sequence reads, clone sequences and assembly contigs with BWA-MEM. Available online at http://arxiv.org/pdf/1303.3997v2) was used to align the reads to the reference sequence.

### 5.5.3 Expression Analysis

(i) Sample Preparation from Leishmania tarentolae

Cells were grown for 2-3 days at 26° C., static (e.g. in 3 ml in a 6-well plates). Whole cell extract (WCE) and cell free culture supernatants corresponding are analyzed by Western blot. For supernatant analysis, grown culture was centrifuged at 1800 g at RT for 5 min and cell free supernatant was transferred to a new tube and mixed with Laemmli dye under reducing or non-reducing conditions. Cell pellets for WCE were washed with 1×PBS, centrifuged again at 1800 g at RT for 5 min and frozen at −80° C. for minimally 30 min. After thawing it again at RT pellet was then resolved in Laemmli (reducing) buffer, boiled again at 95° C. for 10 min and vortexed intensively.

(ii) Expression Analysis by Western Blot

Samples were run on 4-12% Bis-Tris SDS PAGE, using a MOPS running buffer with 200 V for 60 min. Gels were blotted using an Iblot device for 7 min on PVDF membranes. Membranes were blocked for at least 30 min at RT in 10% milk. Primary antibodies (i.e. goat anti-Human IgG-HRP (A6029, Sigma) 1:2000 diluted, mouse anti-Human Kappa Light Chain (K4377, Sigma) 1:5000 diluted or rabbit anti S. pneumoniae serotype 1 polysaccharide (SSI, #16744) 1:100 diluted) were used diluted in 1% milk, 1×PBST for o/n incubation at 4° C. Afterwards, the blot was washed with 1×PBST three times for 5 min before detection with horse reddish peroxidase (HRP) coupled secondary antibodies (anti-mouse polyvalent-HRP (A0412, Sigma) 1:2000 diluted or anti-rabbit-HRP conjugate (Jackson ImmunoResearch #111-035-008) 1:2000 diluted) in 1% milk, lx PBST for 3 h rotating at 30° C., followed by three washes for 5 min in 1×PBST and one component 3,3′,5,5′-tetramethylbenzidine (TMB) substrate staining for colorimetric detection (TMBM-1000-01, Surmodics).

### 5.5.4 Small Scale Expression, Purification of Adalimumab

Host cells were routinely grown in 50 ml culture in BHIH or YEH for 48 h at 26° C. shaking at 140 rpm. Cultures were harvested and centrifuged for 10 min at 1800×g at RT. Media SN was filtered through 0.22 μm filter (Steriflip, SCGP00525) and EDTA (0.5 M pH8) was added to each load in a 1:100 dilution. Media SNs of each strain were subjected to 4 h incubation with 100 μl of proteinA resin (ProteinA-Sepharose 4B Fast Flow, Sigma Aldrich, P9424) per Falcon tube in batch while rotating at RT. After treatment with protein A resin, the samples were centrifuged at 500×g for 5 min, the FT was discarded and the resin was transferred to spin columns. Washes were performed with 3×5 CV using Buffer A (pH 7.2 20 mM Na₂HPO₄, 150 mM NaCl, pH was adjusted with HCl to 7.20) using 500 μl for 100 μl resin; with centrifugation at 1000×g, RT, 1 min between each step. Elution was performed with several CV of Buffer B (0.1 M acetic acid, 100 mM NaCl, pH was adjusted with 1 M NaOH to 3.20) using 100 μl for 100 μl resin, with centrifugation at 1000×g, RT, 1 min between each step (e.g. 3×1 CV and 1×0.5 CV). Elution fractions were pooled and immediately neutralized by adding 100 mM Tris-HCl (1 M pH8). Afterwards, the pooled elutions were buffer exchanged to PBS pH 6 using 2 ml 7K ZebaSpin desalting columns and optionally concentrated using Amicon 0.5 ml 30 K concentrators.

### 5.5.5 Analysis of N-Glycans Released from Purified Proteins and Cells Surfaces by HILIC-UPLC-MS

Enzymatic release of N-glycans from purified proteins was performed using Rapid PNGase F (New England Biolabs) as recommended by the supplier. 8 μl of sample (15 μg of protein) were mixed with 2 μl Rapid Buffer and 1 μl of Rapid PNGase F. The mixture was incubated at 50° C. for 10 min followed by 1 min at 90° C.

Enzymatic release of N-glycans from cell surfaces was performed using PNGase F (New England Biolabs). Cells (grown for 48 or 72 h at 26° C. shaking at 140 rpm) were harvested and washed with PBS by centrifugation for 10 min at 1800×g at RT.50 mg of cell pellet were re-suspended in Glyco Buffer 2 and incubated with 1 μl PNGase F for 1 h at 37° C. and 650 rpm. Cells were again pelleted by centrifugation and 75 μl of the supernatant was dried down in a SpeedVac concentrator. Glycans were resuspended in 10 μl of water. Following release, glycans were directly labeled with procainamide as described previously (Behrens, et al. (2018) Glycobiology 28 (11), pp. 825-831). Briefly, released glycans were mixed with 1 μl acetic acid, 8 μl of a procainamide stock solution (550 mg/ml in DMSO) and 12 μl of a sodium cyanoborohydride stock solution (200 mg/ml in H₂O). Samples were incubated for 60 min at 65° C. and cleaned up using LC-PROC-96 clean up plates (Ludger Ltd) according to the manufacturer's instructions.

Procainamide-labeled N-glycans were analyzed by hydrophilic interaction chromatography-ultra performance liquid chromatography-mass spectrometry (HILIC-UPLC-MS) using am Acquity UPLC System (Waters) with fluorescence detection coupled to a Synapt G2-Si mass spectrometer (Waters). Glycans were separated using an Acquity BEH Amide column (130 Å, 1.7 2.1 mM×150 mM; Waters) with 50 mM ammonium formate, pH 4.4 as solvent A and acetonitrile as solvent B. The separation was performed using a linear gradient of 72-55% solvent B at 0.5 ml/min for 40 min. Fluorescence was detected at an excitation wavelength of 310 nm and a detection wavelength of 370 nm. The Synapt G2-Si mass spectrometer fitted with a Zspray electrospray source was used for mass detection in positive resolution mode using the following parameters: Scan range: m/z 300-3500; scan time: 1 sec; capillary: 2.2 kV; source temperature: 120° C. and sampling cone: 75 V. MassLynx 4.2 (Waters) was used for data acquisition. Data processing and analysis was performed using Unifi 1.9.4.053 (Waters). Glucose units were assigned using a fifth-order polynomial distribution curve based on the retention times of a procainamide-labeled dextran ladder (Ludger Ltd). Glycan structures were assigned based on their m/z values and their retention times and matched against a previously constructed N-glycan library. For individual samples the UPLC was coupled to a Synapt HDMS mass spectrometer using comparable settings.

For a few samples, Waters RapiFluor labelling kit, mostly following the Waters Application Note: <<Quality control and Automation Friendly GlycoWorks RapiFluor-MS N-Glycan Sample Preparation>> that were analyzed using the same instrumentation as the procainamide labelled glycans (RF-MS).

### 5.5.6 DMB Labeling of Neu5Ac and CMP-Neu5Ac

A highly sensitive strategy to quantify the concentration of nucleotide-activated sialic acid by a combination of reduction and fluorescent labeling using the fluorophore 1, 2-diamino-4,5-methylenedioxybenzene (DMB) was applied. The labeling with DMB requires free keto as well as carboxyl groups of the sialic acid molecule. Reduction of the keto group prior to the labeling process precludes the labeling of non-activated sialic acids (Neu5Ac). Since the keto group is protected against reduction by the CMP-substitution, labeling of nucleotide-activated sialic acids is still feasible after reduction. Subsequent combination of the DMB-high-performance liquid chromatography (HPLC) applications allows identification of both total Neu5Ac and modified CMP-sialic acid and quantification in the femtomole range (Galuska, et al. (2010) Anal Chem 82 (11), pp. 4591-4598).

MeOH/Chloroform extraction procedure for L. tarentolae cell pellets was performed on 4 OD of each sample, which were harvested by centrifugation and washed 2× with 1×PBS and frozen. For extraction, pellets were thawed, resuspended in 480 μl MeOH, supplemented with 20 μl water and sonicated in a water bath at RT for 15 min. The samples were spun in a table-top centrifuge at 18000 g and 4° C. for 10 min. The SN was transferred into a glass vial, supplemented with 268 μl chloroform and vortexed. Next, 500 μl H₂O (MS grade) was added and the sample was vortexed again. The MeOH/chloroform/H₂O (1/0.54/1) mixture was spun at 2200 g and RT for 20 min to remove proteins, lipids and DNA in the CHCl3 phase. Approximately half (525 μl) of the upper MeOH/H₂O phase was collected and transferred into Eppendorf tubes, corresponding to extracted material from 2 OD pellet. The samples were dried in a speed-vac, resuspended in 16 μl H₂O and split into two samples of 8 μl that were separately subjected to DMB labeling with and without reduction. As control, Neu5Ac in H₂O was dried in a SpeedVac and dried material was diluted in H₂O, split into two for both labelling procedures. One set of samples was supplemented with 10 μl of ice cold 0.4 M sodium borate buffer pH 6.8 and 2 μl of freshly thawed 2 M borohydride in 0.5 M NaOH (final=0.2 M sodium borate buffer pH 8,8 containing 0.2 M borohydride) and incubated at RT for 2 h (reduced samples). The second set of samples was supplemented with 10 μl of ice cold 0.4 M sodium borate buffer pH 6.8 and 2 μl of 0.5 M NaOH (final=0.2 M sodium borate buffer pH 8,8) and incubated at RT for 2 h (non-reduced samples). Afterwards, samples were dried in a speedVac, resuspended in 3 μl H₂O and subjected to standard DMB labelling using the Takara labeling kit (#4400) according to manufacturer's instructions. Finally, samples were analyzed by RP-C18-LC in duplicates. Quantification was performed using a defined standard curve for which the standard solutions were subjected to incubation in sodium borate buffer (non-reducing) and DMB labeling analogous to the procedure described for non-reduced samples above.

### 6. EXAMPLES

### 6.1 Example 1

To analyze the capability of Leishmania tarentolae to assemble a chromosomal integration construct from multiple DNA fragments by homologous recombination, transfection of the same construct (for expression of a monoclonal antibody, Rituximab) was in parallel attempted by a 1-fragment and a 2-fragment version.

The 1-fragment version (pLMTB5026) contains the coding sequences for light chain, heavy chain and a selection marker (Nourseothricin, ntc) flanked and interspaced by intergenic regions. These intergenic regions are used as spacers (intergenic region, IR) in the construction of synthetic polycistrons, since they are central components of the native polycistronic gene clusters in Leishmania that ensure proper splicing of the pre-mRNA and furthermore are believed to influence gene expression by regulating transcript stability. In addition, the extremities of the DNA fragment contain (600-1000 bp) regions homologous to the L. tarentolae rDNA locus (ssu) in order to integrate the construct into the genome (FIG. 34 in International Publication No. WO2019/002512 A2, incorporated by reference in its entirety herein).

The 2-fragment version contains the same genetic elements, but distributed across two DNA fragments. Fragment P1 (pLMTB5024) contains the coding sequences for light and heavy chain as well as the intergenic regions upstream of these CDS. The 5′ end of the fragment contains the homologous recombination site for integration into the ssu locus. The last 250 bp of the heavy chain CDS (3′ end of P1 construct) are repeated in the first 250 bp of the second fragment (P2; pLMTB5025) in order to allow homologous recombination between the two fragments via their identical sequences. Furthermore, P2 comprises an intergenic region downstream of the heavy chain (CamIR), the selection maker (ntc) followed by another intergenic region (3′UTR=dhfr-ts) and the 3′ homologous recombination site for integration into the ssu locus (FIG. 1A).

The different constructs, either SwaI linearized pLMTB5026 or SwaI linearized pLMTB5024+pLMTB5025, were transfected (Biorad system) into L. tarentolae (St10569). For both versions, viable polyclones were obtained and Western blot analysis of the clones obtained by the 2-fragment version also showed significant monoclonal antibody expression upon detection with light or heavy chain specific antibodies (FIG. 1B). This demonstrates the feasibility of the formation of expression constructs from several DNA fragments.

### 6.2 Example 2

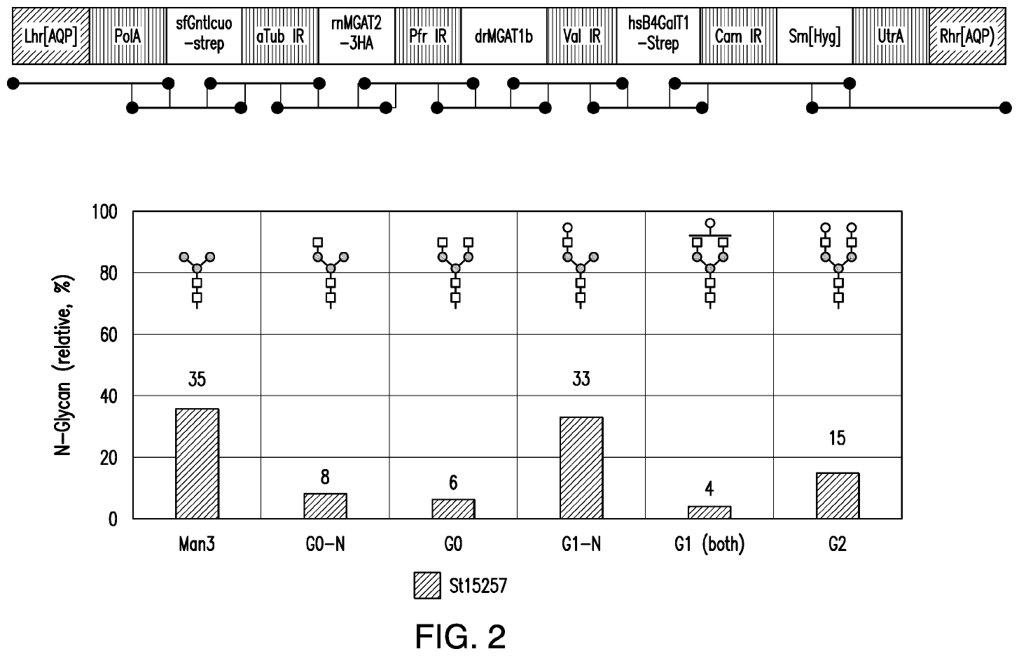

To obtain a cell line expressing four different glycosyltransferases for conversion of the endogenous Man3 to G2 N-glycans (two functionally redundant enzymes for addition of the first glycoengineering step, SfGnt1 and drMGAT1, as well as rnMGAT2 and hsB4GalT1 for further extension to N-glycan “G2”) (International Publication No. WO2019/002512 A2, incorporated by reference in its entirety herein), wild type L. tarentolae (St10569) were transfected with an expression construct formed by homologous recombination of ten DNA fragments. These DNA fragments were designed similar to the previously introduced constructs with intergenic regions interspersing the coding sequences in the assembled synthetic polycistron and a PolI promoter region that is derived from the well described ribosomal DNA locus and supports high-level expression of the counterclockwise-integrated construct. Usually, the aquaporin locus as most protein coding genes in Leishmania is transcribed by PolII, for which specific promoter regions are elusive. Homologous recombination in-between the fragments and between fragments and the aquaporin locus on the genome (AQP) was enabled by 200 bp and 500 bp homologous regions, respectively (FIG. 2, top). Furthermore, the overlaps were planned in a way that allows modular exchange of individual enzymes or intergenic regions by combination of linear fragments from different donor plasmids.

To improve the transfection efficiency for multi-fragment homologous recombinations a new transfection system (Nucleofector) was tested in parallel to the old one (BioRad).

Linear DNA fragments derived from plasmids (pLMTB6855, 6952, 6958, 6807, 6848, 6852, 6811, 6860, 6906, 6861) were transfected into wt L. tarentolae (St10569) by either transfection method 1 using the Biorad system or Transfection method 2 using the Nucleofector system. For both methods, viable polyclones were obtained suggesting the successful recombination of the split selection marker.

The resulting polyclones were analyzed for their engineered N-glycans by RF-MS on whole cell protein level, exemplified for St15257 in FIG. 2 and demonstrated successful glycoengineering up to G2 (16%), which implies that an expression construct covering at least 3 of the enzymes had been assembled by L. tarentolae. This demonstrated the general feasibility of integrating multi-fragment assemblies into L. tarentolae for glycoengineering. For both transfection methods, clones exhibiting similar properties were obtained, demonstrating that the transfection was feasible independent of the applied transfection method. Nevertheless, the clones from the Nucleofector transfection grew up slightly faster than the ones from the BioRad system, suggesting better cell viability after transfection and thus potentially better transfection efficiency.

### 6.3 Example 3

To obtain conversion of the endogenous Man3 to G2 N-glycans in a strain that was previously transfected with a Rituximab expression construct, St12427 was transfected with a second expression construct formed by homologous recombination of ten DNA fragments. The construct encodes for expression of four different glycosyltransferases, i.e. two functionally redundant enzymes for addition of the first GlcNAc, SfGnt1 and drMGAT1, as well as rnMGAT2 and hsB4GalT1 for further extension to G2.

DNA fragments derived from plasmids (pLMTB6950, 6956, 6808, 6849, 6852, 6811, 6816, 6873, 6855, 6861) were transfected into L. tarentolae St12427 by transfection method 2 using the Nucleofector system. Viable polyclones were obtained suggesting the successful recombination of the split selection marker.

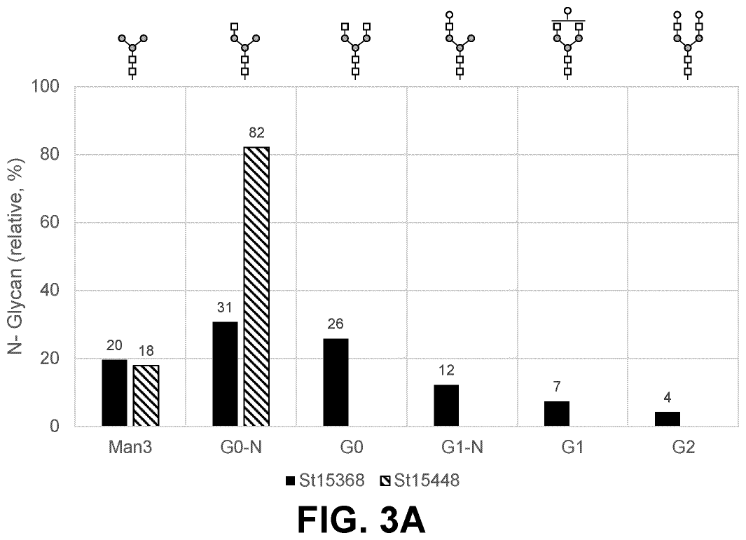

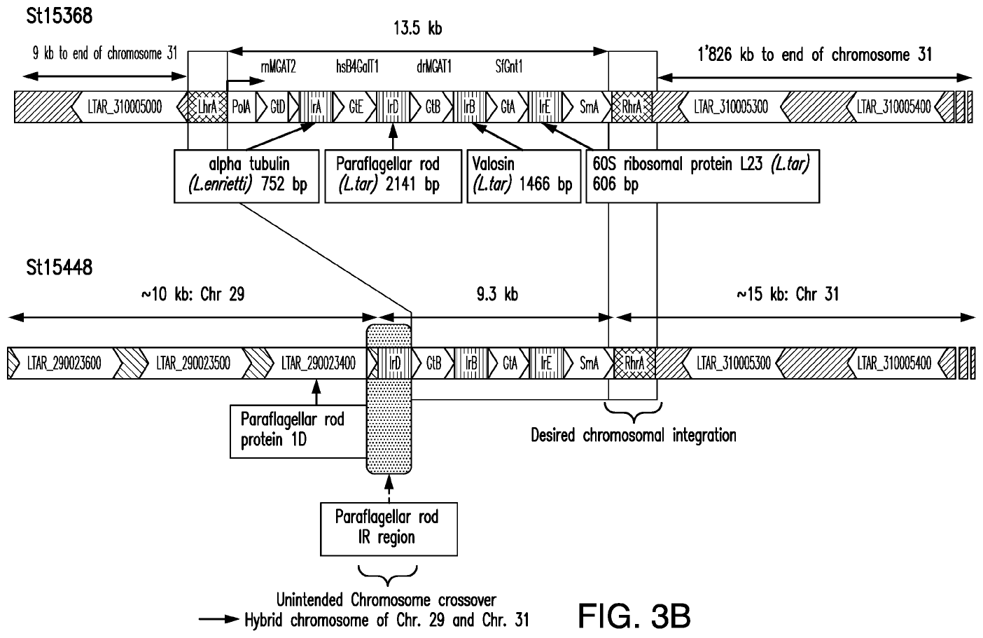

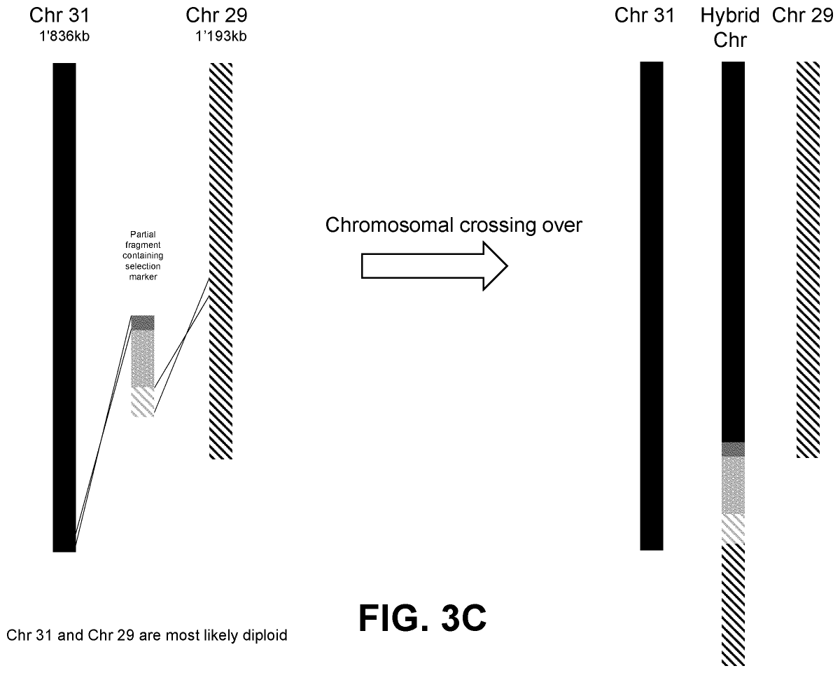

The resulting polyclones were analyzed by RF-MS on whole cell protein level and demonstrated glycoengineering up to G2 (4%) in some strains (St15368), which implies that an expression construct covering at least 3 of the enzymes had been assembled by L. tarentolae. This corroborates the general feasibility of integrating multi-fragment assemblies into L. tarentolae for glycoengineering. Other clones however only showed conversion up to G0-N (e.g. St15448), which suggests an incomplete integration of the construct (FIG. 3A).

In order to assess the genetic composition in strain St15448, gDNA was prepared (Macherey&Nagel NucleoBond® CB100) and subjected to PacBio long read genome sequencing on 2 PacBio SMRT cell (v2.1 chemistry, library preparation according to the manufacturer's specification). Several of the long subreads demonstrated an incomplete integration of the construct that includes only the coding sequences for the glycosyltransferases drMGAT1 and SfGnt1, which both catalyze the addition of the first GlcNAc to Man3. Thus, the obtained sequencing data are in line with the observed phenotype of the N-glycan profile. The data furthermore support that the incomplete integration happened by correct integration of the 3′ end of the construct into the AQP locus on chromosome 31, while instead of 5′ end integration into AQP, the intergenic region PfrIR (native L. tarentolae ˜2 Kb) recombined with the endogenous Pfr expression locus on chromosome 29. By this, a chromosomal crossing over (FIG. 3B) was created. Besides this, also native chromosomes 29 and 31 were detected, most likely in diploid form (data representative cartoon shown in FIG. 3C).

These sequencing data indicate that the wrong homologous recombination was favored over the intended integration locus. This might have been facilitated by the fact that the Pfr intergenic region is long (˜2 Kb) and 100% identical to the native genetic locus and the fact that the AQP locus is very close the telomere of the chromosome.

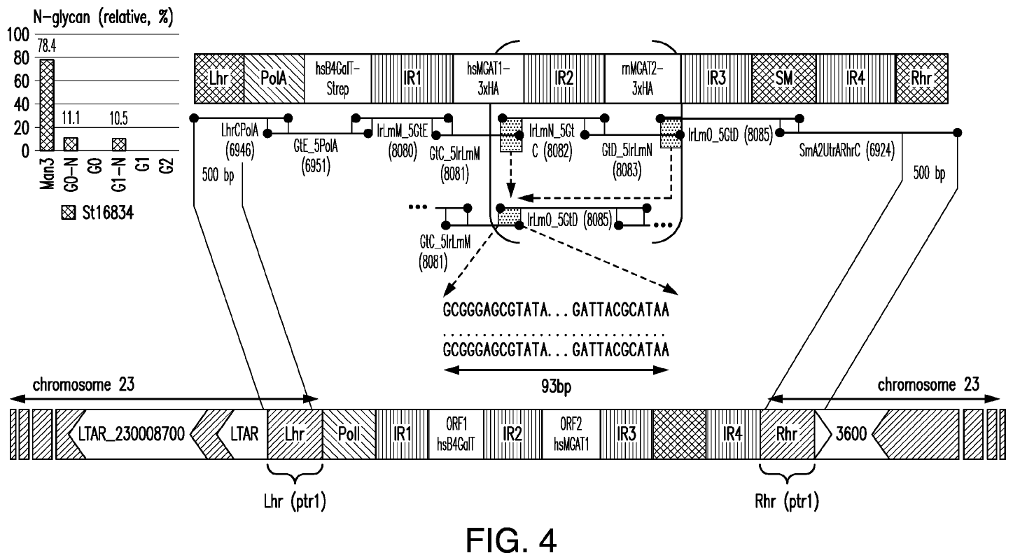

Another example that corroborates the hypothesis that stretches of homology between the fragments should be avoided was identified in a construct where hsB4GalT1-Strep, hsMAGT1-3×HA and rnMGAT2-3×HA were transfected into L. tarentolae wild type background (St10569+pLMTB6946, 6951, 8080, 8081, 8082, 8083, 8085, 6924). In the resulting strain St16834 almost no activity of hsMGAT1 was detectable along with a complete absence of MGAT2 activity (78% M3, 11% G0-N, and 11% G1-N). Long read sequencing with the Nanopore technology revealed that two fragments carrying the rnMGAT2-3×HA had not been integrated into the genomic locus since two adjacent glycosyltransferases were both triple HA-tagged and the nucleotide sequence of the tag region was 100% identical. This demonstrates that a stretch of 93 bp is sufficient for homologous recombination (FIG. 4).

### 6.4 Example 4

The previous examples suggested that the use of homologous sequences between the different fragments for integration as well as between the fragments and the L. tarentolae genome can lead to unwanted homologous recombination. This on one hand necessitates the use of codon-diversified variants or homologs of glycosyltransferases when increasing the gene dosage for a specific N-glycan conversion step. On the other hand, this finding prohibits the repeated use of the previously successfully tested intergenic regions. Data about the exact signals for splicing and mRNA stability in L. tarentolae are not available and thus design of synthetic intergenic regions is currently not feasible. To overcome this limitation, a new set of DNA fragments was designed that use intergenic regions from other Leishmania species and various codon usage variants as wells as different homologues of the different glycosyltransferases.

Genes from L. mexicana, L. donovani, and L. infantum that are believed to be highly expressed have been identified by looking for homologs to L. major genes that were shown to have high relative expression transcript levels (Rastrojo, et al. (2013) BMC Genomics 14, p. 223). The 3′ untranslated region (UTR) of these genes are believed in turn to support high protein expression levels (Murray, et al. (2007) Molecular and Biochemical Parasitology 153 (2), pp. 125-132). To minimize the potential for unwanted homologous recombination, sequences with >80% identity to each other (using cd-hit (Li, et al. (2006) Bioinformatics 22 (13), pp. 1658-1659.) and more than 30 bp identical stretches to the L. tarentolae genome, using blastn (Camacho, et al. (2009) BMC Bioinformatics 10, p. 421), have been excluded.

Protein sequences were back-translated to nucleotide sequences using a custom Python3 script that stochastically selects codons based on the L. tarentolae codon usage frequency while excluding rare codons (frequency <10%). The codon usage has been calculated using cusp (Rice, et al. (2000) Trends in genetics: TIG 16 (6), pp. 276-277) on all annotated L. tarentolae nucleotide coding sequences.

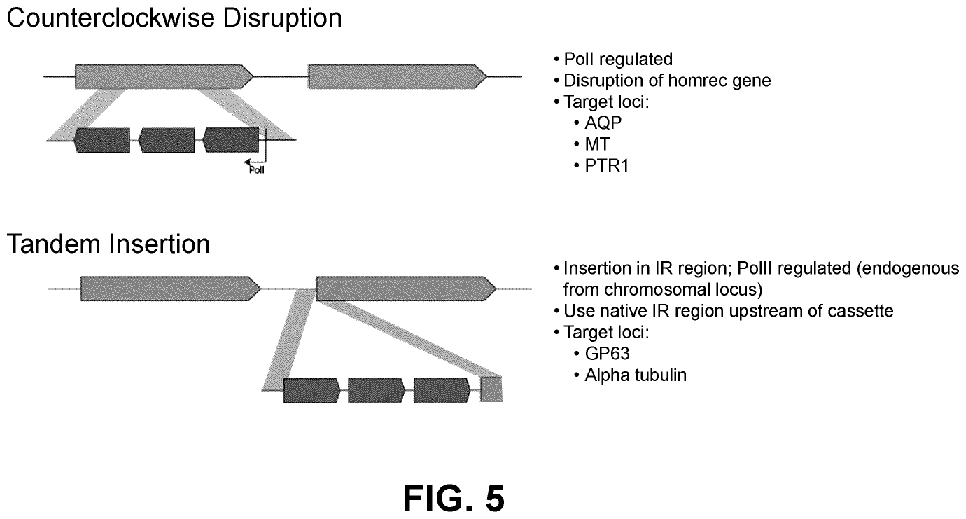

Again, as in the previous multi-fragment homologous recombination constructs, 200 bp overlaps between the fragments and 500 bp homologous regions to the anticipated integration sites were included to allow assembly of the fragments in Leishmania. New integration loci were designed and either used in a “Tandem integration” approach (FIG. 5, bottom), in which the new construct is integrated between the 5′UTR and the coding sequence of a highly expressed or multi-copy gene such as alpha Tubulin (aTub). In this case, no additional promotor region (PolI) is included in the construct and thus the endogenous IR of the target locus will govern the PolII mediated transcription of the first coding sequence of the integration construct. Consequently, the integration construct needs to conclude with an intergenic region at its 3′ end, which spaces the last CDS of the construct and the endogenous gene of the target locus. Alternatively, new loci are used in a “disruptive integration” approach (FIG. 5, top) where the CDS of a target gene is exchanged for the integration construct. In order to profit from the high transcription efficiency of RNA PolI in Leishmania, this integration approach can be paired with use of the PolI promoter region from L. tarentolae and counterclockwise integration. The latter should avoid an imbalanced transcription of neighboring genes that are usually transcribed by PolII (FIG. 5).

In order to test the capability of the IRs from different species to support expression of the glycosyltransferases, a set of four different transfections was performed with the Nucleofector method. Herein, the glycosyltransferases, the selection marker as well as the integration locus (shown example targets GP63 locus) were kept constant and only the intergenic regions were varied. Each transfection construct combined four different IRs from the same species in order to identify whether compatibility is limited to specific species (L. major, L. donovani, L. infantum, L. mexicana). For St17212, these fragments were derived from pLMTB8234, 8235, 8250, 8295, 8297, 8301, 8302, 8303, 6933. For St17311, these fragments were derived from pLMTB 8234, 8235, 8250, 8306, 8307, 8310, 8311, 8312, 6933. For St17176 these fragments were derived from pLMTB 8250, 8334, 8234, 8335, 8235, 8336, 8328, 8330, 6933. For St17180, these fragments were derived from pLMTB 8250, 8322, 8234, 8323, 8235, 8324, 8316, 8318, 6933.

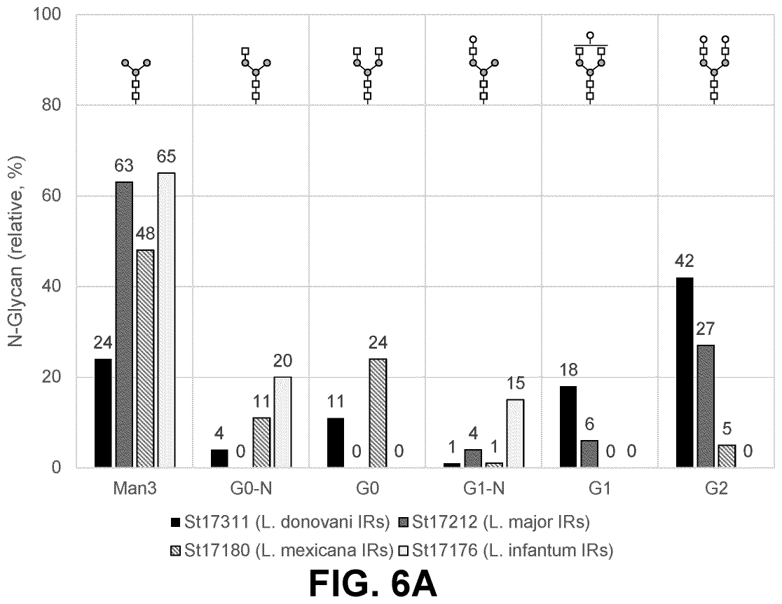

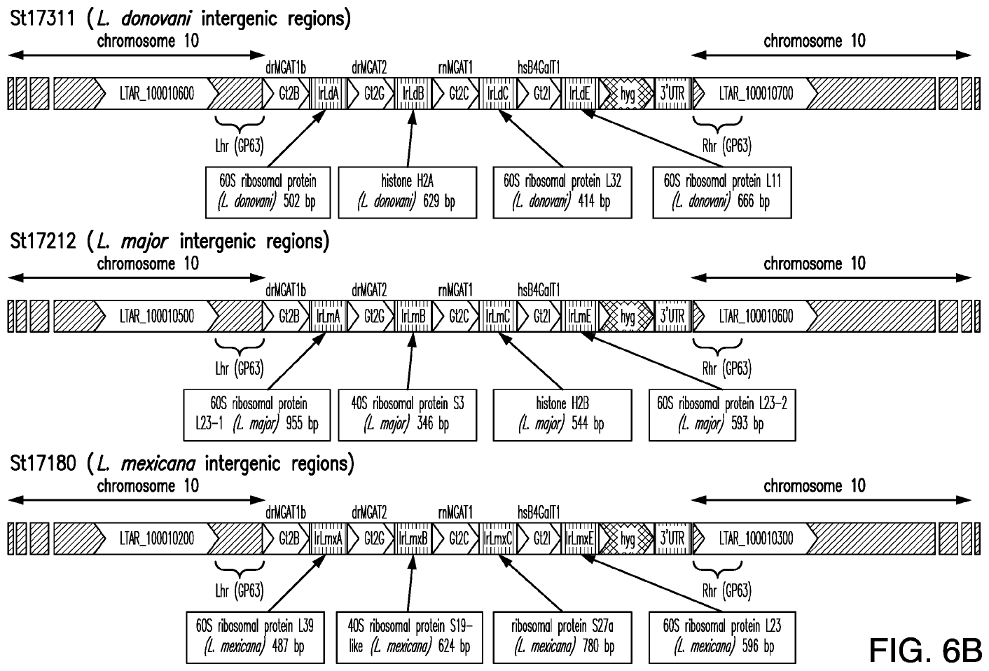

All transfections successfully produced viable clones that were grown in 50 ml shake flask cultures and subjected to N-glycan analysis of the surface protein fraction of L. tarentolae (St17212=LmIR, St17311=LdIR, St17176=LiIR, St17180=LmxIR; FIG. 6A). The N-glycan profiles demonstrated that all four transfections were mostly successful since in the variants containing IRs from L. major, L. donovani, L. mexicana conversion up to G2 and in the variant with L. infantum IRs at least G1-N was detected (FIG. 6A). This demonstrates that intergenic regions from all four Leishmania species can be used to support expression of recombinant glycosyltransferases in L. tarentolae, enabling fully function-customized host cells. The detectable activities for most GTs suggests that the majority of the used IRs are functional and only few, i.e. the ones supporting MGAT2 expression in St17176 were not efficient. Since the contribution of 5′ and 3′ UTRs cannot be clearly distinguished based on the available data, the “non-functional” IRs cannot be unambiguously pinpointed. In addition, heterologous coding sequences could contribute to sequence-mediated mRNA stability.

However, the quite different N-glycan profiles that are observed for the analyzed strains furthermore support the hypothesis that the intergenic regions actually influence the expression level of the genes they flank.

To corroborate correct integration Nanopore sequencing was performed for St17311, St17212 and St17180 and confirmed correct integration for all tested constructs (FIG. 6B).

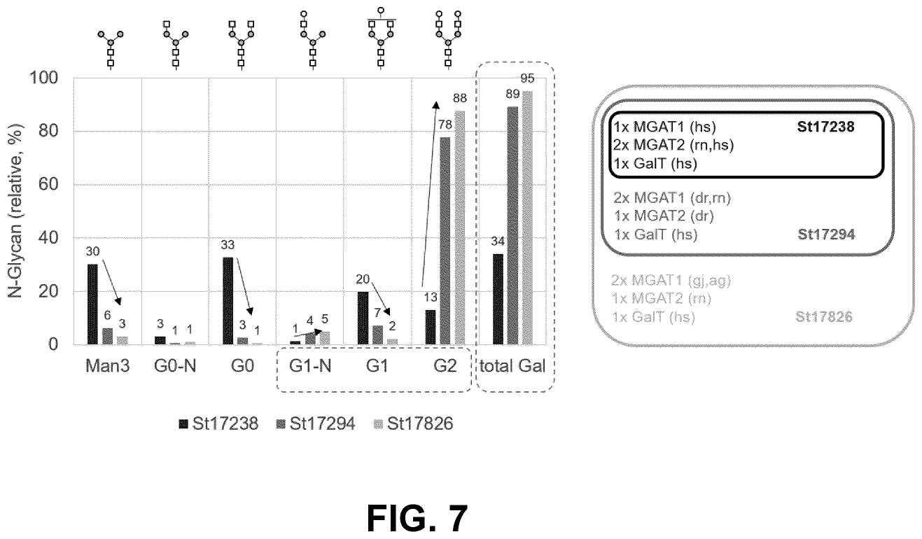

Next, it was assessed whether multiple of these multi-fragment integrations can be combined within one strain to improve the glycoengineering activity and obtain more homogenous N-glycan conversion. For this, St17238 was created by transfection of linearized DNA fragments from plasmids (pLMTB8253, 8313, 8314, 8236, 8315, 8255, 8259, 6940, 8379) targeting the alpha tubulin locus of an Adalimumab expression strain (St15449). Then, in a second transfection a 9-fragment construct derived from plasmids pLMTB8389, 8301, 8234, 8302, 8235, 8303, 8295, 8297, 8392 was integrated into the pfr locus to generate St17294. Last, St17294 was transfected with DNA fragments obtained from plasmids pLMTB8247, 8285, 8237, 8286, 8238, 8287, 8383, 8282, 6936 to obtain a third GT expression construct in the GP63 locus. Comparison of the resulting strain St17826 with its predecessors by N-glycan analysis of surface proteins depicts a step-wise increase in GT activity resulting in almost homogenous G2 N-glycans (88%) for the final strain (FIG. 7). This corroborates the usefulness of the multi-fragment integration method to achieve integration of multiple enzyme copies into the Leishmania genome and that copy number increases of glycosyltransferases can be achieved by using codon diversified enzymes as well as homologs from different species (here: hs, Homo sapiens; rn, Rattus norvegicus, dr, Danio rerio, gj, Gekko japonicus, ag, Anopheles gambiae).

To summarize, for enabling correct multiple homologous recombination events in Leishmania tarentolae, heterologous intergenic regions derived from other Leishmania species were successfully used to site-specifically engineer host cells. Furthermore, these heterologous intergenic regions containing regulatory elements were sufficient to drive splicing and expression of the heterologous coding sequences.

### 6.5 Example 5

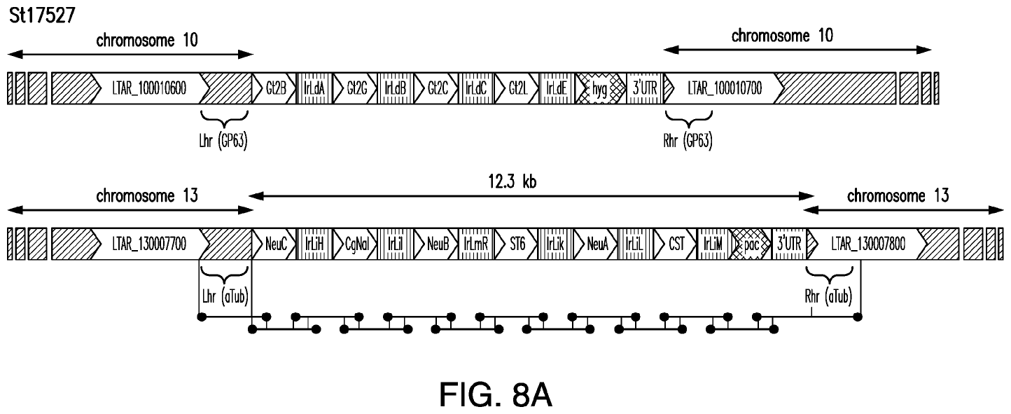

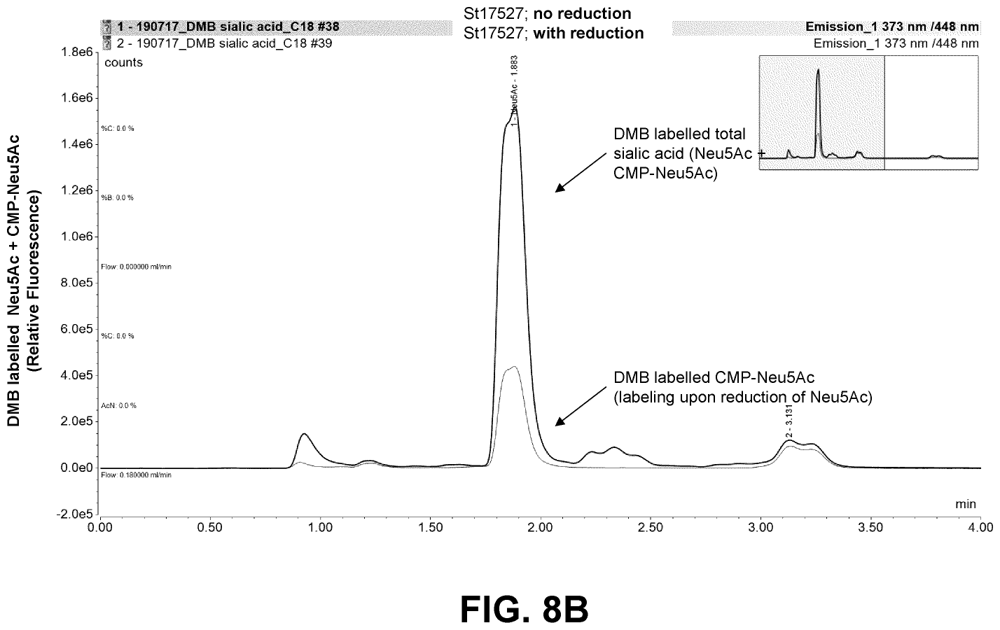

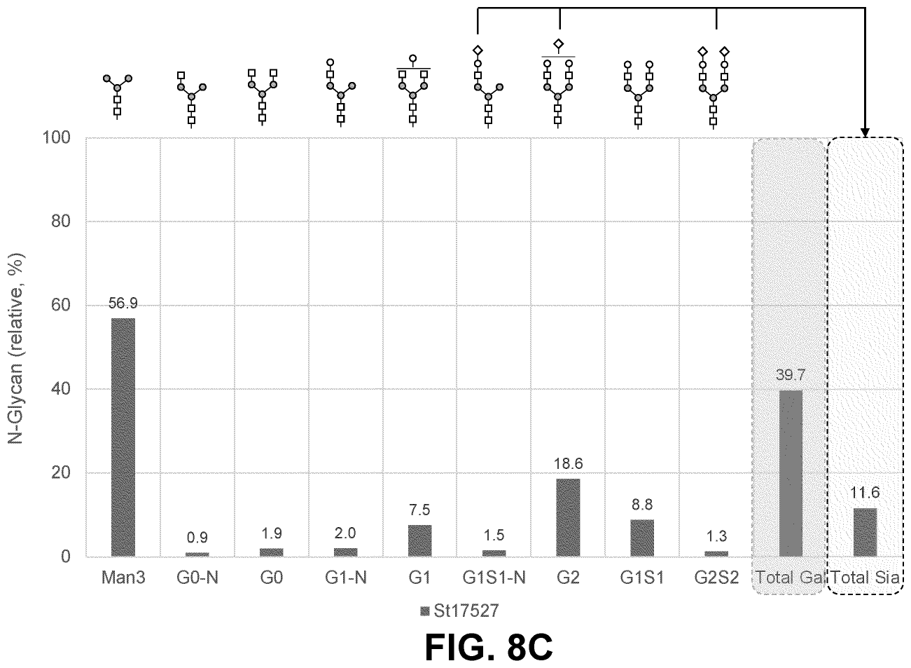

Since the heterologous, non-identical sequences were successfully used for correct multiple recombination events by up to ten DNA fragments, generation of an even larger chromosomal integration cluster by using genetic elements from 13 donor fragments was tested. Importantly, an even more engineered strain was subsequently created, by transfecting an existing strain that is capable of galactosylation, St17311 (described in Example 4 and in FIG. 6A), with an expression construct providing it with the proficiency in generating sialic acid needed for the sialylation engineering of N-glycans (International Publication No. WO2019/002512 A2, incorporated by reference in its entirety herein). DNA elements were used to target the alpha tubulin locus using a tandem insertion strategy. The intended expression cassette contained NeuC_{3×Myc}, IrLiH, CgNal, IrLiI, NeuB_{3xHA}, IrLmR, _{3xHA}mmST6, IrLiK NeuA_{3xHA}, IrLiL, hsCST_{3×myc}, IrLiM and SM (pac) followed by 3′UTR, and was inserted into host cells by transfecting St17311 with thirteen donor fragments excised from pLMTB8443, 8528, 8448, 8529, 8509, 8507, 8505, 8531, 8449, 8532, 8517, 8520, 6939 (FIG. 8A). The resulting strain St17527 was further analyzed for its phenotype: 1) by DMB labeling of production of Neu5Ac and CMP-Neu5Ac (FIG. 8B) and 2) for its engineered N-glycan (FIG. 8C). St17527 produced 0.49 nmol/OD Neu5Ac, 0.17 nmol/OD CMP-Neu5Ac, calculated based on a standard curve (not shown). Moreover, protein linked N-glycans showed a significant amount of sialylated N-glycans, with a total of 11.6% sialylated glycoforms (FIG. 8C). These results demonstrate full functionality of the glycoengineering pathway, indicating that the genetic information is completely inserted in L. tarentolae host cells. This was finally confirmed by PacBio sequencing, clearly showing that the genetic system applied successfully generated fully function-customized L. tarentolae host cells.

### 6.6 Example 6

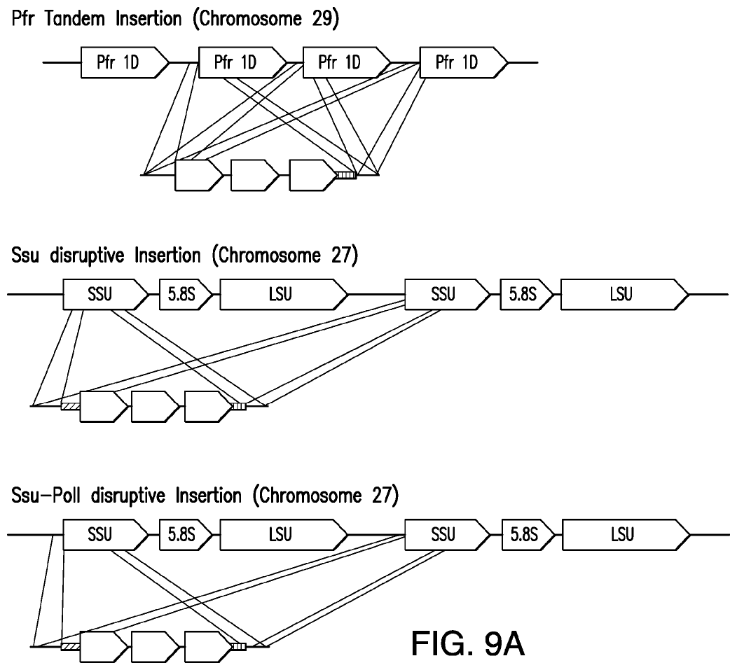

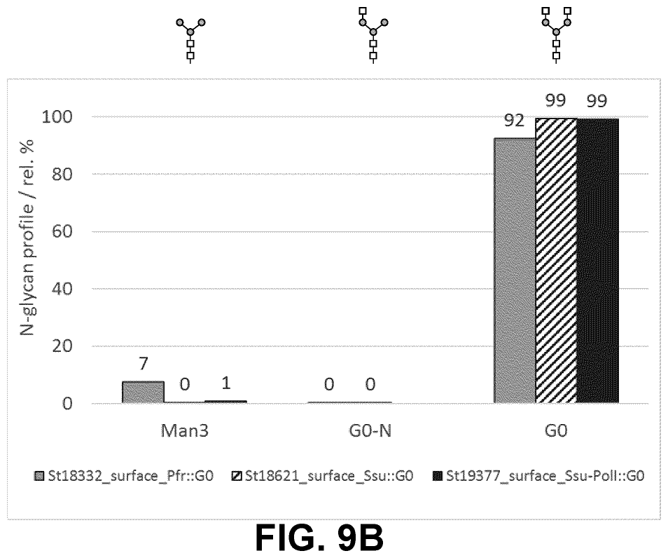

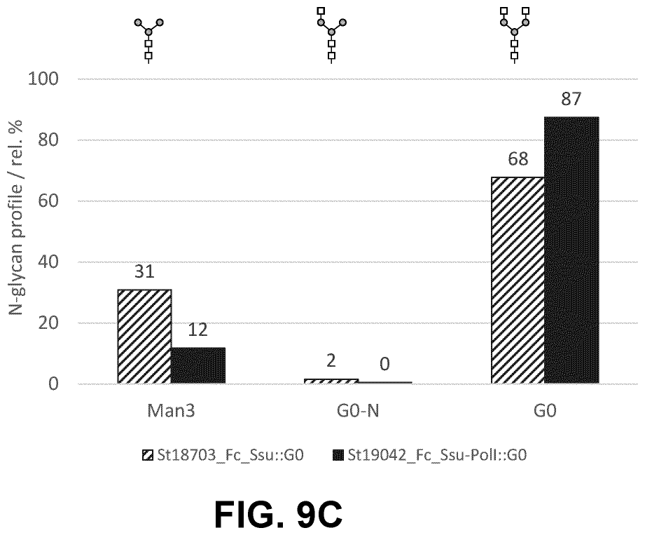

As previously shown in Example 3, the unwanted integration into the native expression site of the paraflagellar rod protein 1D (Pfr) seemed to support high expression on the integrated construct. Thus, it was assessed whether targeting this locus on purpose in a non-disruptive “tandem” integration manner (in comparison to Example 4) would also lead to high level expression and how this compares to expression from the L. tarentolae rDNA locus (“ssu”). Additionally, a different type of integration into the rDNA locus was tested, in which the 5′ integration site was moved 141 bp towards the transcription initiation site of the rDNA locus to the start of the 18S (ssu) coding sequence. For integration into this site (“Ssu-PolI”), protein expression constructs were equipped with an artificial spliced leader acceptor site to ensure correct processing (FIG. 9A). The 3′ integration site was kept the same as that in the case of the “ssu” locus and thus also caused disruption of one of the ssu expression region.

A glycoengineering construct for N-glycan conversion to G0 glycoform, encoding 3 orthologs of MGAT1 (drMGAT1, gjMGAT1 and agMGAT1) as well as 1 ortholog of MGAT2 (drMGAT2), was transfected into the three different loci of WT L. tarentolae strains by transfection of either pLMTB8389, 8301, 8234, 8629, 8238, 8287, 8383, 8282, 8822 (for St18332), pLMTB9299, 8301, 8234, 8629, 8238, 8287, 8383, 8384, 8994 (for St18621) or pLMTB8223, 8381, 8301, 8234, 8629, 8238, 8287, 8383, 8281, 9304. The efficiency of these integrations was compared by comparison of the N-glycan profiles released from Leishmania surface glycoproteins. All three integrations resulted in high level conversion to G0. While integration into the two ssu locus variants performed indistinguishably with 99% G0, integration into the new “Pfr” locus resulted in 92% G0 N-glycans (FIG. 9B). The resulting cell lines showed no significant difference in viability, growth or productivity. Thus, the “Pfr” locus represents an additional high expression locus for Leishmania.

In order to assess further whether there is a difference between the two integration variants for the ssu locus (“Ssu” vs. “Ssu-PolI”, see FIG. 9A), another G0 glycoengineering construct composed of two functional homologs of MGAT1 (sfGNT1, drMGAT1B) and 2 codon usage variants of rat MGAT2 was integrated into the respective loci and combined with a target protein expression construct. The conversion of the sterically more constraint Fc N-glycan of the highly expressed monoclonal antibody serves as a more stringent measure of glycoengineering efficiency.

Strain St18703 was obtained by transfection of St18344 with linearized fragments from plasmids pLMTB9301, 9070, 8568, 9072, 9080, 9082, 9083, 8461 and 8994 into the “Ssu” locus and subsequent transfection of the resulting strain St18625 with an Adalimumab expression construct (pLMTB6737, 8698, 7084, 6681, 6683). For generation of strain St19042, an Adalimumab expression strain St18607 was transfected with linearized fragments from plasmids pLMTB8223, 8564, 9070, 8568, 9072, 9080, 9082, 9083, 8461 and 8994 to obtain integration of the G0 construct into the “Ssu-PolI” locus.

Comparison of the Fc N-glycan profiles of Adalimumab purified from these two strains clearly demonstrated a difference between the integration variants and supports superiority of the “Ssu-PolI” integration variant with conversion to 87% G0 while St18703 only obtained 68% G0 (FIG. 9C). Thus, in order to achieve high level conversion the “Ssu-PolI” locus is the most suitable integration locus, but balanced high level expression can also be achieved by targeting the “Pfr” locus or the alternative “Ssu” integration variant.

### 6.7 Example 7

In order to further explore the opportunities of extending the multiple homologous recombination method, transfection of a genetic element assembled from 25 different donor fragments was attempted. This genetic module combines expression constructs for enzymes of the sialic acid biogenesis pathway (NeuC3×Myc, 3×flagcgNal, NeuB3×HA, NeuA3×HA and 3 codon usage variants of Spinv-A88ST6) with expression constructs for glycosyltransferases. For efficient glycoengineering up to G2, 3 copies of MGAT1, 3 copies of MGAT2 and 2 copies of hsB4GalT were combined by using codon usage variants in the case of hsB4GalT1 and orthologs from different organisms in the cases of MGAT1 and MGAT2. Furthermore, 15 different intergenic regions from 4 previously described Leishmania species were used in this construct to avoid repeated usage of the same sequences. Finally, the selection marker (pac), a 3′UTR as well as flanking sequences for homologous integration in tandem into Pfr locus were included into the construct. Notably, in this case, the selection marker was not situated in the end of the construct, but in between the clusters for glycoengineering.

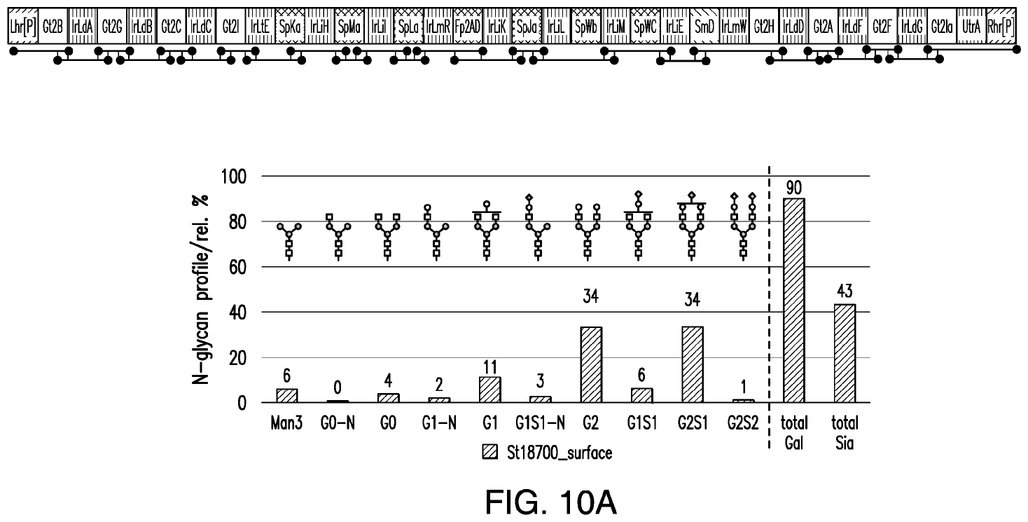

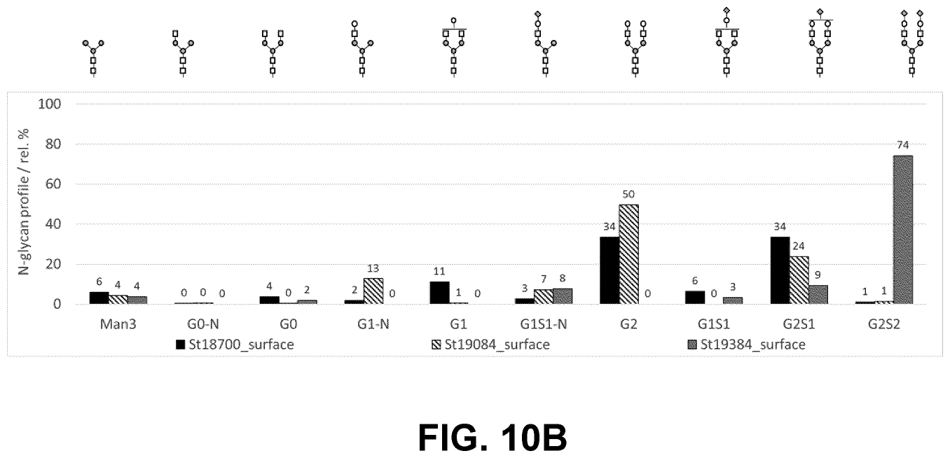

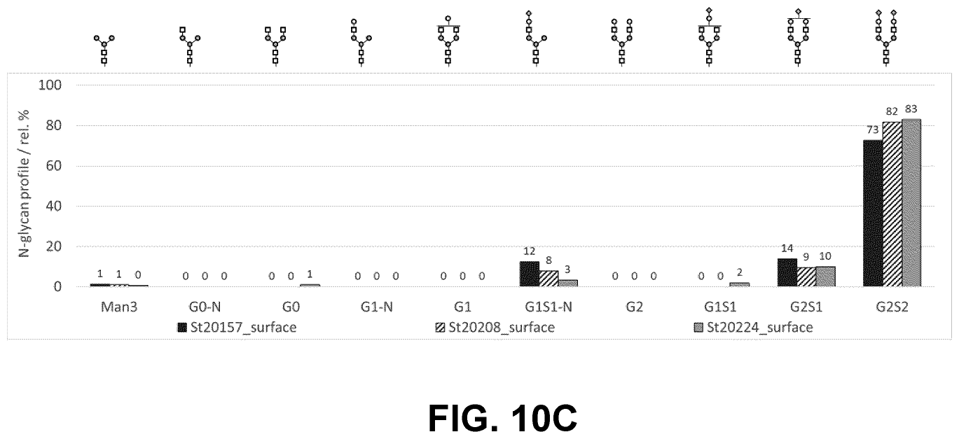

For the integration of this construct, WT L. tarentolae (St18344) were transfected with the twentyfive donor fragments excised from plasmids pLMTB8389, 8310, 8234, 8311, 8235, 8312, 8254, 9220, 8528, 8448, 8529, 8509, 9131, 9132, 8449, 9339, 9340, 8333, 8636, 8313, 8236, 8314, 8379, 8315, 9320 (FIG. 10A).

The phenotype of the resulting strain St18700 was analyzed by N-glycan profiling of its surface glycoproteins. The strain proved to be very proficient in N-glycan conversion up to G2S2, with 90% galactosylated N-glycan species and a total of 43% sialylated N-glycans (FIG. 10A). This suggests full functionality of the glycoengineering pathway, since previously galactosylation of around 90% of the surface glycans could only be obtained by combination of two galactosyltransferase containing modules in different expression loci (compare FIG. 7). Also, long read sequencing of a derived cell line (St19384, see below) confirmed the complete and correct integration of the 30 kbp construct assembled from 25 individual fragments in L. tarentolae. This further underlines the high potential of the novel method in this invention of recombinantly engineering a Leishmania cell that involves homologous recombination of a multitude of DNA fragments.

Next, the resulting strain was further modified by integration of two additional glycoengineering constructs aiming at improving the conversion to G2S2. First, St18700 was transfected with linearized inserts from plasmids pLMTB8391, 8285, 8237, 8286, 8238, 8287, 8383, 8281 and 8821, which constitute another glycoengineering module with a different codon usage variant of hsB4GalT1, a codon usage variant of rnMGAT2 and two additional orthologs of MGAT1 from different organisms. This modification led to a marked increase in G2 in the surface N-glycan profile of the resulting strain St19084 (FIG. 10B).

In order to improve sialylation of theses N-glycan species, an additional glycoengineering module containing sfGNT1, a functional homolog of MGAT1, as well as additional codon usage variants of MGAT1 from zebrafish and MGAT2 from rat for boosting the conversion of Man3 to higher modified N-glycan variants was transfected into St19084. Additionally, the module contained another ortholog of the sialyltransferase ST6, ₛₜᵣₑₚCMAS and the sialic acid transporter CST for improved activation and transfer of sialic acid to the protein acceptors. For this transfection, linearized fragments from plasmids pLMTB8223, 8564, 8567,8568,8823,8599, 9486, 8488, 8447, 8490 and 9205 were transfected into the parental strain to obtain strain St19384. In this case, two plasmids, 8223 and 9205 had to be cut with alternative restriction enzymes (HindIII+SmiI or BglII+SmiI) to create the wanted overhangs for homologous recombination. By this transfection, high sialylation of the N-glycans released from surface glycoproteins with 74% G2S2 was obtained (FIG. 10B).

In order to confirm that all the previously mentioned glycoengineering modules were correctly integrated into this highly modified strain, high molecular weight gDNA from St19384 was prepared and subjected to Nanopore sequencing. By this, correct integration of the three glycoengineering modules in the targeted loci could be confirmed.

This example ultimately demonstrated the high potential of genetic modifications of L. tarentolae by the techniques described herein for multiple homologous recombination events, in each of the subsequent rounds of engineering. In total 45 linearized fragments were transfected into the cells in order to establish 3 different glycoengineering modules comprised of 7 orthologs or functional homologs of MGAT1, 5 orthologs or codon usage variants of MGAT2, 3 codon usage variants of hsB4GalT1, 4 codon usage variants or orthologs of ST6 and 6 enzymes of the sialic acid biosynthesis pathway. Thus, avoiding repetition of identical sequences in the coding as well as non-coding regions of the constructs as described in detail in previous examples, allows extensive modification of Leishmania cells without unwanted recombinations.

Confirmation of the reproducibility of extensive strain engineering like the one described before was obtained with strains St20157, St20208 and St20224, which each contain 3 glycoengineering constructs as well as an O-glycosylation knock-out (see International Application entitled “Glycoengineering Using Leishmania Cells” filed even date herewith) and are derived from the common parental strain St19084 (FIG. 10C).

### 6.8 Example 8

Assembly of a Hybrid Prokaryotic Gene Cluster on an Escherichia coli Cosmid in Leishmania tarentolae.

The recombinant expression in E. coli of the Streptococcus pneumoniae serotype 1 capsular polysaccharide as lipid-linked oligosaccharide (LLO) requires 10 exogenous genes. Seven genes are present as a cluster in S. pneumoniae, while three genes are present elsewhere in its genome. As the orthologues of these three genes are widespread in prokaryotes, genes from Plesiomonas shigelloides 017, a closer E. coli relative, were chosen, as they were elsewhere proven efficient in their function when recombinantly expressed in E. coli.

The aim of the experiment is to obtain a functional hybrid cluster cloned into the E. coli-compatible cosmid pLAFR1 (Vanbleu, E. et al. (2004) DNA Seq 15 (3): 225-227) by exploiting L. tarentolae recombination machinery's ability to assemble DNA fragments sharing homologies at their ends. pLAFR1 contains a tetracycline resistance for its selection, and a broad range origin of replication for Enterobacteriaceae. pGVXN775 is a derivative of pLAFR1 in which a multiple cloning site, a constitutive promoter J23114 (Anderson collection), and a transcriptional terminator have been introduced. Its linearization via AsiSI and XhoI allows insertion of DNA fragments between the constitutive promoter and the terminator.

Eleven fragments were designed as described in FIG. 11A and their synthesis was performed at GENEWIZ Germany GmbH. The following aspects have been taken into account for the design: a) Fragments length should not exceed 2000 bp in order to increase synthesis speed, b) overlap between fragments and between fragment and vector to be 200 bp for optimal homologous recombination efficiency.

The final construct is designed to contain a selection marker usable in L. tarentolae to be inserted together with the necessary 5′ and 3′ regulatory elements at the 3′ of the gene cluster. The selection marker gene, i.e. streptothricin acetyl transferase (sat), which confer resistance to nourseothricin (NTC), is intact only if recombination takes place as it is split in two fragments. The selection marker cassette is flanked by the restriction enzyme BsiWI for its excision.

Two sets of recombinations were performed. For one set, called “pLAFR_Sp1” set, the 10 genes needed for the biosynthetic pathway and the selection marker cassette are split into nine fragments and recombined into pGVXN775. The total size of the insert is 14789 bp. The product should be able to convert E. coli in a S. pneumoniae serotype 1 LLO producer. A second set, called “pLAFR_SM” set, is a control strategy in which the selection marker cassette is split into 2 fragments and recombined into pGVXN775. The total size of the insert is 2956 bp.

Three different transfections in Leishmania tarentolae St10569 have been carried out as summarized in Table 2. For all the transfections, the Biorad transfection method has been followed. In transfection #1 cells have been co-transfected with the AsiSI-XhoI-linearized pGVXN775 and the 9 fragments needed for “pLAFR_Sp1”. Transfection #2 differs from the previous, as the target vector is not linearized. In transfection #3 not linearized pGVXN775 is co-transfected with the two fragment needed for the “pLAFR_SM” set.

Growing cultures were analyzed by colony PCR using DreamTaq DNA Polymerase (Thermo Fisher Scientific) according to manufacturer's instructions. PCR A uses oligonucleotides o4949 and o4978 and is a positive control for lysis; PCR B uses oligonucleotides o229 and o6775 and amplifies the intersection between pGVXN775 and the 5′ part of the inserted “pLAFR_Sp1” set; PCR C uses oligonucleotides o228 and o6045 and amplifies the intersection between the 3′ part of the inserted “pLAFR_Sp1” set and pGVXN775; PCR D uses oligonucleotides o6517 and o6521 and amplifies an internal sequence of the “pLAFR_Sp1” set; PCR E uses oligonucleotides o5976 and o6776 and amplifies the intersection between the 3′ part of the inserted “pLAFR_SM” and pGVXN775. PCRs A, B, C, D have been applied to cells from transfections #1 and #2, while PCRs A and E have been applied to cells from transfection #3. A polyclone is defined positive when all the applied PCRs yield the expected product band. The number of positive polyclones per transfection is reported in Table 2.

DNA was isolated from eight PCR-positive L. tarentolae polyclones from transfection #1 (polyclones 1.1, 1.2, 1.3, 1.4, 1.5, 1.6, 1.7, 1.8), three PCR-positive polyclones from transfection #2 (polyclones 2.1, 2.2, 2.3), and two PCR-positive polyclones from transfection #3 (polyclones 3.1, 3.2) using Macherey Nagel NucleoSpin plasmid Miniprep kit, following manufacturer's instructions for low copy E. coli plasmid isolation. The eluted material likely contains episomal and chromosomal DNA. The DNA was used to transform chemical competent E. coli DH5α via heat shock. The transformed colonies were plated on LB-Agar tetracycline plates. The growing colony are able to express the tetracycline-resistance cassette encoded in pGVXN775. One single colony per polyclone was inoculated in liquid LB tetracycline, and plasmid DNA was isolated using Macherey Nagel NucleoSpin plasmid kit, according to manufacturer's instructions.

E. coli DH5α cells transformed with all plasmids derived from polyclones from transfections #1 and #2 were assessed for their capability to express S. pneumoniae serotype 1 polysaccharide as lipid linked oligosaccharide (LLO). 5 mL LB tetracycline cultures have been grown o/n at 37° C. in shaking culture tubes. The volume corresponding to 2 ODs was centrifuged, the pellet resuspended in Lammli buffer, incubated 10 minutes at 95° C., cooled, supplemented with 2 μL of Proteinase K from Tritirachium album ≥800 units/mL (Sigma-Aldrich P4850), incubated at 55° C. for 1 hour, then at 70° C. for 10 minutes. 10 μL (corresponding to 0.1 OD) have been loaded on 4-12% Bis-Tris polyacrylamide gels for an SDS page. After the run, gel material has been transferred onto a blotting membrane and was detected with an antibody specific for S. pneumoniae serotype 1 polysaccharides. The Western blot on a part of the strains is depicted in FIG. 11B. For transfection #1 8 out of 8 clones showed S. pneumoniae type 1 polysaccharide production, for transfection #2 1 out of 3, as reported in Table 2. The production of the glycan indicates that a correct assembly took place.

To confirm the correct assembly, and to investigate non-producers, restriction analyses were carried out using standard restriction enzymes (Thermo Fisher Scientific). Plasmids from polyclones 1.1, 1.7, 2.1 and 2.2 were separately digested with BstBI or BsiWI. The tested LLO-positive clones show the expected restriction pattern. A non-producer from transfection #2 (polyclone 2.2) shows also the right pattern, while the non-producer 2.1 shows a negative pattern (FIG. 11C). Polyclones 3.1 and 3.2 from transfection 3 were digested with Sad. The expected pattern for selection marker cassette insertion is observed, as deduced from the comparison with an identical plasmid obtained by conventional cloning, pLMTB6412 (FIG. 11D).

Plasmids from polyclones 1.1 and 2.2 were further investigated via primer walking Sanger sequencing of the entire cosmid. Polyclone 1.1 shows 100% sequence identity to the expected 35038-bp construct. Polyclone 2.2 showed right restriction pattern but lack of activity. The sequencing shows 99% identity, a GG is deleted causing a frameshift in wbzG, inactivating the production of polysaccharide. The inserted selection marker cassette and its intersections with pGVXN775 of the plasmid from polyclone 3.2 were analyzed via Sanger sequencing, confirming 100% identity to expected sequence.

Plasmid derived from polyclone 1.1 has been digested via BsiWI in order to remove the selection marker cassette, and religated. The obtained plasmid retains its S. pneumoniae serotype 1 glycan production activity.

A correct gene assembly has been achieved in 100% of the analyzed plasmids when nine fragments and a linearized vector have been co-transfected (transfection #1). The efficiency of the assembly on a circularized vector seems to be inferior but still a valid option in case of absence of suitable restriction sites as 1 case out of 3 yielded a phenotypic positive with the pLAFR_Sp1 set (transfection #2) and 2 out of 2 positives with the pLAFR_SM set (transfection #3).

TABLE 2
Summary of the analyses on the assembled plasmids

| | | | Polyclones | | | |
|---|---|---|---|---|---|---|
| | | | for | | | |
| Transfection | Transfected DNA | Colony PCR | plasmid | Phenotypic | Restriction | |
| ID | fragments | (nₚₒₛᵢₜᵢᵥₑ/n_{tested)} | isolation | test | analysis | Sequencing |
| #1 | pLMTB7382(SEQ1), | 14/14 | 1.1 | Positive | Positive | Whole |
| | 7383(SEQ2), | | | | | plasmid, |
| | 7384(SEQ3), | | | | | 100% |
| | 7385(SEQ4), | | | | | correct |
| | 7386(SEQ5), | | 1.2 | Positive | NA | NA |
| | 7387(SEQ6), | | 1.3 | Positive | NA | NA |
| | 7388(SEQ7), | | 1.4 | Positive | NA | NA |
| | 7390(SEQ8), | | 1.5 | Positive | NA | NA |
| | 7391(SEQ9), | | 1.6 | Positive | NA | NA |
| | linearized | | 1.7 | Positive | Positive | NA |
| | pGVXN775 | | 1.8 | Positive | NA | NA |
| #2 | pLMTB7382(SEQ1), | 8/9 | 2.1 | Negative | Negative | NA |
| | 7383(SEQ2), | | 2.2 | Negative | Positive | Whole |
| | 7384(SEQ3), | | | | | plasmid, 2- |
| | 7385(SEQ4), | | | | | bp |
| | 7386(SEQ5), | | | | | insertion |
| | 7387(SEQ6), | | 2.3 | Positive | NA | NA |
| | 7388(SEQ7), | | | | | |
| | 7390(SEQ8), | | | | | |
| | 7391(SEQ9), | | | | | |
| | circular pGVXN775, | | | | | |
| #3 | pLMTB7392(SEQA), | 10/10 | 3.1 | NA | Positive | NA |
| | 7393(SEQB), | | 3.2 | NA | Positive | Insert, |
| | circular pGVXN775 | | | | | 100% |
| | | | | | | correct |

### 7. EQUIVALENTS

The viruses, nucleic acids, methods, host cells, and compositions disclosed herein are not to be limited in scope by the specific embodiments described herein. Indeed, various modifications of the viruses, nucleic acids, methods, host cells, and compositions in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

Lengthy table referenced here
US20230048847A1-20230216-T00001
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20230048847A1). An electronic copy of the table will also be available from the USPTO upon request and payment of the fee set forth in 37 CFR 1.19(b)(3).

What is claimed:

1. A method of recombinantly engineering a Leishmania cell comprising
(a) introducing two or more DNA fragments into the Leishmania cell, and
(b) incubating the Leishmania cell to allow homologous recombination of the DNA fragments,
wherein a first DNA fragment of the two or more DNA fragments comprises a 5′ homologous region and/or a 3′ homologous region; wherein the 5′ homologous region is homologous to a 3′ homologous region of a second DNA fragment of the two or more DNA fragments or the 3′ homologous region of the first DNA fragment is homologous to a 5′ homologous region of the second DNA fragment; and
wherein the nucleotide sequences of the first and the second DNA fragments outside the homologous region(s) are not homologous to each other; are not homologous to a sequence in the Leishmania cell's genome; and/or have no homologies within the respective DNA fragment.

2. The method of claim 1, wherein each of the two or more DNA fragments comprises a 5′ homologous region and/or a 3′ homologous region; wherein the 5′ homologous region of the each of the two or more DNA fragments is homologous to a 3′ homologous region of another one of the two or more DNA fragments or the 3′ homologous region of the each of the two or more DNA fragments is homologous to a 5′ homologous region of another one of the two or more DNA fragments; and wherein the nucleotide sequences outside the homologous regions in each DNA fragment are not homologous to each other; are not homologous to a sequence in the Leishmania cell's genome; and/or have no homologies within the respective DNA fragment.

3. The method of any one of claims 1 to 2, wherein the two or more DNA fragments, optionally after the two or more DNA fragments are recombined with each other, are suitable for integration into a chromosome of the Leishmania cell.

4. The method of claim 3, wherein the two or more DNA fragments, optionally after the two or more DNA fragments are recombined with each other, are integrated into the chromosome of the Leishmania cell.

5. The method of claim 4, wherein the two or more DNA fragments are integrated in tandem into the paraflagellar rod protein (Pfr) locus.

6. The method of claim 4, wherein the two or more DNA fragments are integrated at the start of the 18S coding region (Ssu-PolI).

7. The method of any one of claims 1 to 2, the two or more DNA fragments, before and/or after recombination with each other, are not integrated in a chromosome of the Leishmania cell.

8. The method of claim 7, wherein the homologous recombination of the two or more DNA fragments results in a circular plasmid.

9. The method of any one of claims 1 to 8, wherein the nucleotide sequence of the first DNA fragment outside the homologous region is at least 10 nucleotides, 20 nucleotides, 30 nucleotides, 40 nucleotides, 50 nucleotides, 100 nucleotides, 200 nucleotides, 300 nucleotides, 400 nucleotides, 500 nucleotides, 600 nucleotides, 700 nucleotides, 800 nucleotides, 900 nucleotides, 1000 nucleotides, 2000 nucleotides, 5000 nucleotides, 10000 nucleotides, 15000 nucleotides, or 20000 nucleotides, 25000 nucleotides, 30000 nucleotides, 35000 nucleotides, 40000 nucleotides, 45000 nucleotides, or at least 50000 nucleotides in length.

10. The method of any one of claims 1 to 9, wherein the nucleotide sequence of the second DNA fragment outside the homologous region is at least 10 nucleotides, 20 nucleotides, 30 nucleotides, 40 nucleotides, 50 nucleotides, 100 nucleotides, 200 nucleotides, 300 nucleotides, 400 nucleotides, 500 nucleotides, 600 nucleotides, 700 nucleotides, 800 nucleotides, 900 nucleotides, 1000 nucleotides, 2000 nucleotides, 5000 nucleotides, 10000 nucleotides, 15000 nucleotides, or 20000 nucleotides, 25000 nucleotides, 30000 nucleotides, 35000 nucleotides, 40000 nucleotides, 45000 nucleotides, or at least 50000 nucleotides in length.

11. The method of any one of claims 1 to 10, wherein the nucleotide sequences of all of the two or more DNA fragments outside the homologous region are at least 10 nucleotides, 20 nucleotides, 30 nucleotides, 40 nucleotides, 50 nucleotides, 100 nucleotides, 200 nucleotides, 300 nucleotides, 400 nucleotides, 500 nucleotides, 600 nucleotides, 700 nucleotides, 800 nucleotides, 900 nucleotides, 1000 nucleotides, 2000 nucleotides, 5000 nucleotides, 10000 nucleotides, 15000 nucleotides, or 20000 nucleotides, 25000 nucleotides, 30000 nucleotides, 35000 nucleotides, 40000 nucleotides, 45000 nucleotides, or at least 50000 nucleotides in length.

12. The method of any one of claims 1 to 11, wherein the homologous recombination of the DNA fragments results in a nucleotide sequence that is 50 nucleotides to 100 nucleotides, 100 nucleotides to 500 nucleotides, 500 nucleotides to 1000 nucleotides, 1000 nucleotides to 5000 nucleotides, 5000 nucleotides to 10000 nucleotides, 10000 nucleotides to 15000 nucleotides, 15000 nucleotides to 20000 nucleotides, 20000 nucleotides to 25000 nucleotides, 25000 nucleotides to 30000 nucleotides, 30000 nucleotides to 35000 nucleotides, 35000 nucleotides to 40000 nucleotides, 40000 nucleotides to 45000 nucleotides, 45000 nucleotides to 50000 nucleotides, 50000 nucleotides to 55000 nucleotides, 55000 nucleotides to 60000 nucleotides, 60000 nucleotides to 65000 nucleotides, 65000 nucleotides to 70000 nucleotides, 70000 nucleotides to 75000 nucleotides, or 75000 nucleotides to 80000 nucleotides in length.

13. The method of any one of claims 1 to 12, wherein the 5′ homologous region and/or the 3′ homologous region of the first DNA fragment is at least 10 nucleotides, 20 nucleotides, 30 nucleotides, 40 nucleotides, 50 nucleotides, 100 nucleotides, 150 nucleotides, 200 nucleotides, 250 nucleotides, 300 nucleotides, 350 nucleotides, 400 nucleotides, 450 nucleotides, or at least 500 nucleotides in length.

14. The method of any one of claims 1 to 13, wherein the 5′ homologous region and/or the 3′ homologous region of the second DNA fragment is at least 10 nucleotides, 20 nucleotides, 30 nucleotides, 40 nucleotides, 50 nucleotides, 100 nucleotides, 150 nucleotides, 200 nucleotides, 250 nucleotides, 300 nucleotides, 350 nucleotides, 400 nucleotides, 450 nucleotides, or at least 500 nucleotides in length.

15. The method of any one of claims 1 to 14, wherein the 5′ homologous region and/or the 3′ homologous region of all of the two or more DNA fragments is at least 10 nucleotides, 20 nucleotides, 30 nucleotides, 40 nucleotides, 50 nucleotides, 100 nucleotides, 150 nucleotides, 200 nucleotides, 250 nucleotides, 300 nucleotides, 350 nucleotides, 400 nucleotides, 450 nucleotides, or at least 500 nucleotides in length.

16. The method of any one of claims 1 to 15, wherein the 5′ homologous region and/or the 3′ homologous region of the first DNA fragment is at most 500 nucleotides, 550 nucleotides, 600 nucleotides, 650 nucleotides, 700 nucleotides, 750 nucleotides, 800 nucleotides, 850 nucleotides, 900 nucleotides, 950 nucleotides, 1000 nucleotides, 1200 nucleotides, 1400 nucleotides, 1600 nucleotides, 1800 nucleotides, 2000 nucleotides, 2200 nucleotides, 2400 nucleotides, 2600 nucleotides, 2800 nucleotides, 3000 nucleotides, 3200 nucleotides, 3400 nucleotides, 3600 nucleotides, 3800 nucleotides, 4000 nucleotides, 4200 nucleotides, 4400 nucleotides, 4600 nucleotides, 4800 nucleotides, or at most 5000 nucleotides in length.

17. The method of any one of claims 1 to 16, wherein the 5′ homologous region and/or the 3′ homologous region of the second DNA fragment is at most 500 nucleotides, 550 nucleotides, 600 nucleotides, 650 nucleotides, 700 nucleotides, 750 nucleotides, 800 nucleotides, 850 nucleotides, 900 nucleotides, 950 nucleotides, 1000 nucleotides, 1200 nucleotides, 1400 nucleotides, 1600 nucleotides, 1800 nucleotides, 2000 nucleotides, 2200 nucleotides, 2400 nucleotides, 2600 nucleotides, 2800 nucleotides, 3000 nucleotides, 3200 nucleotides, 3400 nucleotides, 3600 nucleotides, 3800 nucleotides, 4000 nucleotides, 4200 nucleotides, 4400 nucleotides, 4600 nucleotides, 4800 nucleotides, or at most 5000 nucleotides in length.

18. The method of any one of claims 1 to 17, wherein the 5′ homologous region and/or the 3′ homologous region of all of the two or more DNA fragments is at most 500 nucleotides, 550 nucleotides, 600 nucleotides, 650 nucleotides, 700 nucleotides, 750 nucleotides, 800 nucleotides, 850 nucleotides, 900 nucleotides, 950 nucleotides, 1000 nucleotides, 1200 nucleotides, 1400 nucleotides, 1600 nucleotides, 1800 nucleotides, 2000 nucleotides, 2200 nucleotides, 2400 nucleotides, 2600 nucleotides, 2800 nucleotides, 3000 nucleotides, 3200 nucleotides, 3400 nucleotides, 3600 nucleotides, 3800 nucleotides, 4000 nucleotides, 4200 nucleotides, 4400 nucleotides, 4600 nucleotides, 4800 nucleotides, or at most 5000 nucleotides in length.

19. The method of any one of claims 1 to 18, wherein the 5′ homologous region of the first DNA fragment and the 3′ homologous region of the second DNA fragment have at least 20%, 25%, 30%, 35%, 40%, 45%, 50%, 55%, 60%, 65%, 70%, 75%, 80%, 85%, 90%, 95%, or 100% sequence identity.

20. The method of any one of claims 1 to 18, wherein the 3′ homologous region of the first DNA fragment and the 5′ homologous region of the second DNA fragment have at least 20%, 25%, 30%, 35%, 40%, 45%, 50%, 55%, 60%, 65%, 70%, 75%, 80%, 85%, 90%, 95%, or 100% sequence identity.

21. The method of any one of claims 1 to 20, wherein the two or more DNA fragments are introduced by transfection.

22. The method of any one of claims 1 to 20, wherein the two or more DNA fragments are introduced concurrently.

23. The method of any one of claims 1 to 22, wherein the number of DNA fragments is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 37, 39, 40, 41, 42, 43, 44, 45, 46, 47, 48, 49, or 50.

24. The method of any one of claims 1 to 23, wherein the nucleotide sequences of the two or more DNA fragments outside the homologous region are selected from a group consisting of intergenic regions (IRs), untranslated regions (UTRs), and open reading frames (ORFs) encoding polypeptides.

25. The method of claim 24, wherein the IRs, UTRs and ORFs are devoid of homologous sequences within itself, and/or homologous sequences to one another.

26. The method of any one of claims 1 to 25, wherein the nucleotide sequences of the two or more DNA fragments outside the homologous region encode the same polypeptide.

27. The method of claim 26, wherein the Leishmania cell is capable of expressing multiple copies of the same polypeptide.

28. The method of any one of claims 26 and 27, wherein the method increases the expression level of the polypeptide.

29. The method of any one of claims 1 to 26, wherein the homologous recombination of the DNA fragments results in a nucleotide sequence comprising at least 50%, 60%, 70%, 80%, 90% or 100% of genetic information encoded by the two or more DNA fragments.

30. The method of any one of claims 1 to 29, wherein undesired crossing out and/or crossing over occurs in at most 0.01%, 0.05%, 0.1%, 0.5%, 1%, 2%, 3%, 4%, 5%, 6%, 7%, 8%, 9% or at most 10% of the Leishmania cells over a period of at least 1 day, 2 days, 3 days, 4 days, 5 days, 6 days, 7 days, 8 days, 9 days, or at least 10 days.

31. The method of any one of claims 1 to 29, wherein undesired crossing out and/or crossing over occurs in at most 0.01%, 0.05%, 0.1%, 0.5%, 1%, 2%, 3%, 4%, 5%, 6%, 7%, 8%, 9% or at most 10% of the Leishmania cells over at least 1, 2, 3, 4, 5, 6, 7, 8, 9, or at least 10 cell divisions.

32. The method of any one of claims 1 to 31, wherein the Leishmania cell is Leishmania tarentolae.

33. A Leishmania cell recombinantly engineered using the method of any one of claims 1 to 32.

34. The Leishmania cell of claim 33, wherein the Leishmania cell is recombinantly engineered using the method repeatedly.

35. The Leishmania cell of any one of claims 33 to 34, wherein the Leishmania cell is Leishmania tarentolae.

36. A kit comprising one or more containers and instructions for use, wherein said one or more containers comprise the Leishmania cell of any one of claims 33 to 35.

37. A method of making a polypeptide comprising (a) culturing the Leishmania cell of any one of claims 33 to 35 under suitable conditions for polypeptide production; and (b) isolating the polypeptide.

38. The method of claim 37, wherein the method further comprises introducing a nucleotide sequence encoding the polypeptide.

39. A polypeptide produced by the method of any one of claims 37 to 38.
